(19) United States
(12) Patent Application Publication
(10) Pub. No.: US 2012/0244192 A1
(43) Pub. Date: Sep. 27, 2012

(54) SUSTAINED RELEASE FORMULATIONS OF PEPTIDOMIMETIC DRUGS AND USES THEREOF

(76) Inventor: Gary P. Cook, Westford, ME (US)

(21) Appl. No.: 13/266,718

(22) PCT Filed: Apr. 26, 2010
(86) PCT No.: PCT/US10/32421

§ 371 Date: Jun. 14, 2012

Related U.S. Application Data
(60) Provisional application No. 61/172,975, filed on Apr. 27, 2009.

(51) Int. Cl. A61K 38/08; A61P 9/10; A61P 29/00; A61K 9/14; A61P 27/02

(52) U.S. Cl. 424/400; 514/21.7; 514/20.8

(57) ABSTRACT

The invention provides sustained release formulations comprising a C5a receptor antagonist. In certain embodiments the sustained-release formulations include microparticles that comprise a complement C5aR antagonist and a biodegradable polymeric matrix. Methods of treatment comprising the sustained release formulations of the invention are also provided.

This application claims the benefit of the filing date of U.S. Provisional Patent Application No. 61/172,975, filed Apr. 27, 2009, the contents of which are hereby incorporated by reference.

This patent disclosure contains material which is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights.

Throughout this application, patent applications, published patent applications, issued and granted patents, texts, and literature references are cited. For the purposes of the United States and other jurisdictions that allow incorporation by reference, the disclosures of these publications are incorporated by reference into this application.

### FIELD OF THE INVENTION

The invention relates to drug delivery of peptide and peptidomimetic drugs. In certain aspects the invention relates to sustained release formulations and uses thereof. In other aspects, the invention relates to sustained release formulation using microparticles comprising a biodegradable polymer. In certain aspects, the invention relates to sustained release formulations for ophthalmic use.

### BACKGROUND OF THE INVENTION

Sustained release of therapeutic agents for defined and/or long periods of time is desirable, as it reduces frequency of the administration of therapeutic agents. The invention provides sustained release formulation of peptidomimetic therapeutic agents.

### SUMMARY OF THE INVENTION

The present invention provides a microparticle which comprise any of the active agents as described herein, their pharmaceutically acceptable salts, homologues, isomers, or any combination thereof and a biodegradable polymer.

The present invention provides sustained release compositions which comprise any of the active agents as described herein, their pharmaceutically acceptable salts, homologues, isomers, or any combination thereof.

The invention is directed to sustained and/or controlled formulations for administration of one or more therapeutic/active agents through the use of one polymeric particles, such as microparticles, which may effectively treat or improve one or more undesirable conditions associated or due to complement activation, and/or where the inhibition of the complement system leads to relief of symptoms of such a condition. The present sustained release formulations comprise a pharmaceutically acceptable polymeric composition and are formulated to release and/or maintain therapeutic levels of one or more pharmaceutically active agents throughout an extended period of time, such as more than: one week, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 32 weeks, 33 weeks, 34 weeks, 35 weeks, 36 weeks, 37 weeks, 38 weeks, 39 weeks, 40 weeks, and in certain embodiments for a period of time of one year or more. The present sustained release formulations comprise a pharmaceutically acceptable polymeric composition and are formulated to release and/or maintain therapeutic levels of one or more pharmaceutically active agents throughout an extended period of time, such as about: one week, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 32 weeks, 33 weeks, 34 weeks, 35 weeks, 36 weeks, 37 weeks, 38 weeks, 39 weeks, 40 weeks, and in certain embodiments for a period of time of one year or more. The present sustained release formulations comprise a pharmaceutically acceptable polymeric composition and are formulated to release and/or maintain therapeutic levels of one or more pharmaceutically active agents throughout an extended period of time, such as: one week, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 32 weeks, 33 weeks, 34 weeks, 35 weeks, 36 weeks, 37 weeks, 38 weeks, 39 weeks, 40 weeks, and in certain embodiments for a period of time of one year or more. Compared to known sustained release formulations which comprise a peptide as an active agent, the inventive formulations disclosed herein continue to release the active agent for more than four months after administering of the inventive sustained release formulation.

In certain embodiments, the agents released by the formulations of the invention are released in therapeutic amounts so as to treat or improve or ameleriate a condition associated with complement activation. In certain embodiments, the formulations of the invention may include other active agents which are beneficial for the treatment of conditions associated with complement activation.

The inventive compositions comprise a polymeric component and an active agent component. In certain embodiments, the polymeric and active agent components form a microparticle. As described herein, the polymeric component can comprise one or more biodegradable polymers, one or more biodegradable copolymers, one or more non-biodegradable polymers, and combinations thereof. The therapeutic component of the inventive formulation comprises one or more active agents. The present compositions are effective to provide a therapeutically effective dosage(s) of the agent or agents to the organ, tissue or region to be treated, for example but not limited to, directly to a region of the eye, to treat, prevent, and/or reduce one or more symptoms of one or more undesirable, for example but not limited to ocular conditions. Thus, with a single administration, the active agent will be made available at the site where they are needed and will be maintained at effective concentrations for an extended period of time, rather than subjecting the patient to repeated injections or, in the case of self-administered drops, ineffective treatment with only limited bursts of exposure to the active agent or agents or, in the case of systemic administration, higher systemic exposure and concomitant side effects or, in the case of non-sustained release dosages, potentially toxic transient high tissue concentrations associated with pulsed, non-sustained release dosing.

In certain aspects the invention provides a sustained-release microparticle comprising: a complement antagonist or a pharmaceutically acceptable acid addition salt thereof, and a biodegradable and biocompatible polymeric matrix. In certain aspects the invention provides a sustained-release microparticle comprising: a complement C5aR antagonist or a pharmaceutically acceptable acid addition salt thereof, and a biodegradable and biocompatible polymeric matrix. In certain embodiments, the antagonist is a peptidomimetic.

In certain aspects the invention provides a sustained-release microparticle comprising: a small molecule complement C5aR antagonist or a pharmaceutically acceptable acid addition salt thereof; and a biodegradable and biocompatible polymeric matrix.

In certain aspects the invention provides a sustained-release microparticle comprising: a peptide, peptide like, peptidic, peptide derived complement C5aR antagonist or a pharmaceutically acceptable acid addition salt thereof; and a biodegradable and biocompatible polymeric matrix.

In certain embodiments of the sustained-release microparticle of the invention, the biodegradable and biocompatible polymeric matrix is selected from the group consisting of poly(glycolic acid), poly-D,L-lactic acid, poly-L-lactic acid, copolymers of the foregoing, poly(aliphatic carboxylic acids), copolyoxalates, polycaprolactone, polydioxonone, poly(ortho carbonates), poly(acetals), poly(lactic acid-caprolactone), polyorthoesters, poly(glycolic acid-caprolactone), polyanhydrides and polytyrosine (polypeptide polymers) or similar “pseudo”-poly(amino acids) polymers.

In certain embodiments of the sustained-release microparticle of the invention, the antagonist comprises 1 to 20-30% wt. % of the microparticle. In certain embodiments of the sustained-release microparticle of the invention, the antagonist comprises about 2 to 20 wt. % of the microparticle. In certain embodiments of the sustained-release microparticle of the invention, the antagonist comprises about 3 to 15 wt. % of the microparticle.

In certain embodiments of the sustained-release microparticle of the invention, the microparticle ranges in size from 1 to 100 microns. In certain embodiments of the sustained-release microparticle of the invention, the microparticle ranges in size from 25 to 65 microns.

In certain embodiments, the microparticle is formulated in a liquid injection vehicle. In certain embodiments, the microparticle is formulated in an aqueous liquid injection vehicle. In certain embodiments, the aqueous liquid injection vehicle is selected from the group consisting of physiological solution and an aqueous solution of carboxymethyl cellulose with a surfactant.

In certain aspects, the invention provides methods for using the microparticles of the invention. In certain embodiments, the method comprise administering the microparticles of the invention to a subject so as to treat a disease or conditions associated with the complement system or complement activation. In certain aspects, the subject is need of treatment. In certain aspects, the microparticle is administered by any suitable means such as but not limited to intramuscular injection, subcutaneous injection, local administration to the disease site, for example by injection, periocular injection, intraocular injection, any combination, or any other suitable method.

In certain embodiments, the complement antagonist is any of the compounds disclosed in United States Patent Application Publication No. 2006/0183883, or a pharmaceutically acceptable acid addition salt thereof. In certain embodiments, the complement antagonist is JPE1375 or a pharmaceutically acceptable acid addition salt thereof.

In certain aspects, the invention provides a sustained release composition comprising an antagonist to the complement cascade. In certain aspects, the invention provides a sustained release composition comprising C5aR antagonist to the complement cascade. In certain aspects, the invention provides a sustained release composition comprising a peptide or peptidomimetic C5aR antagonist to the complement cascade.

In certain embodiments, the sustained release composition comprises a minimum of 3, 5-10, 6-8, >8 weight % of peptide or peptidomimetic C5aR antagonist to the complement cascade. In certain embodiments, the sustained release composition comprises a minimum of 3, 5, 5-10, 6-8, >8 to about 15 weight % of peptide or peptidomimetic C5aR antagonist to the complement cascade.

In certain embodiment, the peptide or peptidomimetic C5aR antagonist to the complement cascade is one of those disclosed in United States Patent Application Publication No. 2006/0183883.

In certain embodiments, the sustained release compositions of the invention include a biodegradable material. In certain embodiments, the biodegradable material is poly(d,l-lactide-co-glycolide PLGA. In certain embodiments, the PLGA material has any of the following characteristics or any combination thereof 50/50 PLGA to PLA, preferably 85/15 to 95/5 PLGA; IV 0.20 to 1.3, preferably 0.35-0.70. In certain embodiments, ester, acid or blended polymers are suitable for use in the invention.

In certain embodiments, the sustained release composition of the invention aggregates in aqueous media. In certain embodiments, the sustained release composition of the invention settles in aqueous media.

In certain embodiments, the sustained release composition of the invention has a specific density of less than (<), greater than (>), or equal to (=) 1.0.

In certain embodiments, the sustained release composition of the invention is injectable as a suspension through a 27 gauge or smaller syringe needle assembly. In certain embodiments, the sustained release composition of the invention is injectable as a 5%, 10%, 20% 30% 40% weight suspension through a 27 gauge or smaller syringe needle assembly.

In certain embodiments, the sustained release composition of the invention provides a minimum of 1 month release of the therapeutic agent. In certain embodiments, the sustained release composition of the invention provides a minimum of 1 month release of the therapeutic agent and release of the agent for up to 2 months, 3 months, 4 months, 5 months, or 6 months. In certain embodiments, the sustained release composition of the invention provides a minimum of 1 month exposure of the therapeutic agent at a therapeutically effective or relevant concentration.

In certain embodiments, the sustained release composition of the invention provides a 1-2, 2-3, 4-6, 5-6, >6, 6-8, 8-10, 10-12 month release and/or achieves therapeutic levels of the therapeutic agent over such time periods. In certain embodiments, the sustained release composition of the invention provides exposure of the therapeutic agent at a therapeutically effective or suitable concentration for 1-2, 2-3, 4-6, 5-6, or >6 months, for example 6-8, 8-10, or 10-12 months.

In certain aspects, the invention provides use of any of the compositions described herein for the treatment of ophthalmic diseases. In certain aspects, the invention provides use of any of the compositions described herein for treatment of age related macular degeneration. In certain aspects, the invention provides use of any of the compositions described herein for the treatment of geographic atropy. In certain aspects, the invention provides use of any of the compositions described herein for the treatment of diabetic retinopathy. In certain aspects, the invention provides use of any of the compositions described herein for the treatment of conditions related to ischemic or reperfusion injury. In certain aspects, the invention provides use of any of the compositions described herein for the treatment of inflammatory disease. In certain aspects, the invention provides use of any of the compositions described herein for the treatment of systemic inflammatory disease, local inflammatory disease, or a combination thereof. In certain embodiments, local or localized inflammatory disease or condition can be treated by administering by any suitable methods locally to a diseased site a composition of the invention, whereby the local active agent concentration is maintained at therapeutic levels, while there is limit systemic exposure to the therapeutic agent. In certain aspects, the invention provides use of any of the compositions described herein for the treatment of C5aR mediated inflammatory disease. In certain aspects, the invention provides use of any of the compositions described herein for the treatment of a C5aR-mediated ophthalmic inflammatory disease. In certain aspects, the invention provides use of any of the compositions described herein for the treatment of ophthalmic inflammatory disease.

In certain aspects, the invention provides a sustained release pharmaceutical composition comprising as an active agent a compound as represented by formula I, formula II, formula IV, formula V, formula VI, as described in U.S. Ser. No. 10/564,788 (U.S. Patent App. Publication No. 20060183883), and U.S. Ser. No. 11/814,050 (U.S. Patent App. Publication No. 20080161232), the contents of which applications are incorporated herein, or a pharmaceutically acceptable acid addition salt thereof and a poly(d,l-lactide-co-glycolide) (PLGA) polymer.

In certain embodiments, the active agent and the PLGA polymer form a microparticle.

The invention provides a pharmaceutical composition for sustained release of an active agent, as represented by formula I, formula II, formula IV, formula V, formula VI, as described in U.S. Ser. No. 10/564,788, and U.S. Ser. No. 11/814,050 the contents of which applications are incorporated herein or a pharmaceutically acceptable acid addition salt thereof, the composition comprising (a plurality of) microparticles which comprise: (a) the active agent as described herein or a pharmaceutically acceptable salt thereof, and (b) a biodegradable polymer.

A pharmaceutical composition for sustained release of an active agent as represented by formula I, formula II, formula IV, formula V, formula VI, as described in U.S. Ser. No. 10/564,788, and U.S. Ser. No. 11/814,050 the contents of which applications are incorporated herein or a pharmaceutically acceptable acid addition salt thereof, the composition comprising (a plurality of) microparticles which consist essentially of: (a) the active agent as represented by formula I, formula II, formula IV, formula V, formula VI, as described in U.S. Ser. No. 10/564,788, and U.S. Ser. No. 11/814,050 the contents of which applications are incorporated herein or a pharmaceutically acceptable acid addition salt thereof, and (b) a biodegradable polymer.

A pharmaceutical composition for sustained release of an active agent as represented by formula I, formula II, formula IV, formula V, formula VI, as described in U.S. Ser. No. 10/564,788, and U.S. Ser. No. 11/814,050 the contents of which applications are incorporated herein or a pharmaceutically acceptable acid addition salt thereof, the composition prepared by any of the methods described herein.

A mircroparticle for sustained release of an active agent as represented by formula I, formula II, formula IV, formula V, formula VI, as described in U.S. Ser. No. 10/564,788, and U.S. Ser. No. 11/814,050 the contents of which applications are incorporated herein or a pharmaceutically acceptable acid addition salt thereof, the microparticle prepared by any of the methods described herein.

In certain embodiments, the polymer is selected from the group consisting of tyrosine or “pseudo”-poly(amino acids) based polymer, poly-anhydride polymer, poly(d,l-lactic acid), poly(l-lactic acid), poly(glycolic acid), copolymers of the foregoing including poly(d,l-lactide-co-glycolide) (PLGA), poly(caprolactone), poly(orthoesters), poly(acetals) and poly(hydroxybutryate).

In certain embodiments, the solvent is ethyl acetate, benzyl alcohol, methylene chloride, DMSO, or methanol.

In certain embodiments, the active agent is a C5a receptor antagonist. In certain embodiments, the active agent has the following structure: Hoo-Phe-Orn-Pro-hle-Pff-Phe-NH2 (referred to as JPE1375), Ac-Phe-Orn-Pro-hle-Pff-Phe-NH2, Ac-Phe-Orn-Pro-hle-Mcf-Phe-NH2, Ac-Phe-Orn-Pro-hle-Bta-Phe-NH2, Ac-Phe-Orn-Pro-hle-Trp-Phe-NH2, Ac-Phe-Orn-Pro-cha-Trp-Phe-NH2, Ac-Phe-[Orn-Pro-cha-Trp-Orn], Ac-Phe-[Orn-Pro-cha-Trp-Nva], Ac-Phe-[Orn-Pro-cha-Trp-Nle], Ac-Phe-[Orn-Pro-cha-Trp-Ile], Ac-Phe-[Orn-Pro-cha-Trp-Leu], Ac-Phe-[Orn-Pro-cha-Trp-Hle], Ac-Phe-[Orn-Pro-cha-Trp-Chg], Ac-Phe-[Orn-Pro-cha-Trp-Cha], Ac-Phe-[Orn-Pro-cha-Trp-Hch], Ac-Phe-[Orn-Pro-cha-Trp-Eag], Ac-Phe-[Orn-Pro-cha-Trp-Phe], Ac-Phe-[Orn-Pro-cha-Trp-Thi], Ac-Phe-[Orn-Pro-cha-Trp-1Ni], or Ac-Phe-[Orn-Pro-cha-Trp-2Ni].

In certain embodiments, the polymer is PLGA. In certain embodiments, the PLGA polymer has mole ration of lactide:glycolide of 100:0, 85:15, 65:35, or 50:50. In certain embodiments, the PLGA polymer has mole ration of lactide:glycolide of 85:15.

In certain embodiments, the PLGA polymer has specific viscosity, suitable to make the microparticles of the invention.

In certain embodiments, the PLGA polymer has specific MW, suitable to make the microparticles of the invention.

In certain embodiments, the microparticle size is between 10 microns and 1000 microns, or is between 10 microns and 100 microns, or is between 20 microns and 45 microns. In certain embodiments, the microparticles have a median size distribution of about 25, 35, 45, 55, 65, 75, 85, 95, 105, or 115 microns. In this context, “about” is defined as +/−5-10 microns variation above or below the specified sizes.

In certain embodiments, about 50% to about 90% of the microparticles have a size between 25 microns and 45 microns, or between 20 microns and 65 microns.

In certain embodiments, at least about 50%, 60%, 70%, 80%, or 90% of the microparticles have a size between 20 microns and 45 microns, or between 25 microns and 65 microns.

In certain embodiments, the microparticle comprises about 5% to about 15% of the active agent. In certain embodiments, the microparticle comprises about 5% to about 85% PLGA polymer. In certain embodiments, the microparticles comprise less than about 0.08% to about 0.025% residual solvent, or any other component which is not the active agent or the biodegradable polymer, e.g., the PLGA polymer.

In certain embodiments, the microparticle further comprises a pharmaceutical excipient.

In certain embodiments, the compositions have a limited burst release.

In certain embodiments, the compositions provide sustained release of, and/or maintain therapeutic levels of, the active agent throughout a period of at least about: 90 days to about 250 days, 90 days to about 240 days, 90 days to about 230 days, 90 days to about 220 days, 90 days to about 210 days, 90 days to about 200 days, 90 days to about 190 days, 90 days to about 180 days, 90 days to about 170 days, 90 days to about 160 days, 90 days to about 150 days, 90 days to about 140 days, 90 days to about 130 days, 90 days to about 120 days, 90 days to about 110 days, or 90 days to about 100 days.

In certain embodiments, the compositions provide sustained release of, and/or maintain therapeutic levels of, the active agent throughout a period of about 90, 100, 110, 120, 130, 140, 150, 160, 170, 180, 190, 200, 210, 220, 230, 240, or 250 days.

In certain embodiments, the compositions are syringable through a narrow gauge needle of 25 gauge, 27 gauge, 29 gauge, 30 gauge.

In certain aspects, the invention provides a kit for administering a composition, the kit comprising a composition of the invention and a sterile diluent, or a liquid injection vehicle.

In certain aspects, the invention provides an ophthalmic pharmaceutical composition for sustained release of an active agent as described herein or a pharmaceutically acceptable salt thereof, the composition comprising microparticles which comprise, or in certain embodiments consist essentially of, or in certain embodiments consist of: (a) the active agent as described herein or pharmaceutically acceptable acid addition salts, and (b) PLGA, wherein in certain embodiments the microparticles have any one of, or any combination of any of the following characteristics: microparticle size from 20 to 45 microns, or 25 to 65 microns, such that the microparticles are syringable through a 27 gauge needle or narrower needle, or the active agent is about 5% to about 15%, 3%, 5%, 6-8%, or more than 8% to about 15% by weight of the microparticle, or the microparticle has residual solvent levels of less than about 0.08% to about 0.25%, or wherein the initial burst release is limited, or the microparticle releases the active agent for a period of at least four months, five months, six months, seven months, eight months, nine months, ten months, or eleven months to about twelve months.

In certain aspects, the invention provides a plurality of microparticle(s) for sustained release of an active agent as described herein or a pharmaceutically acceptable acid addition salt thereof, the microparticles comprising: (a) the active agent and (b) a biodegradable polymer.

In certain embodiments, the polymer is selected from the group consisting of tyrosine or “pseudo”-poly(amino acids) based polymers, poly-anhydride polymer, poly(d,l-lactic acid), poly(l-lactic acid), poly(glycolic acid), copolymers of the foregoing including poly(d,l-lactide-co-glycolide) (PLGA), poly(caprolactone), poly(orthoesters), poly(acetals) and poly(hydroxybutryate).

In certain embodiments, the active agent is JPE1375, which has the following structure: Hoo-Phe-Orn-Pro-hle-Pff-Phe-NH2, and the polymer is PLGA.

In certain aspects, the invention provide a method for making microparticles for use in a pharmaceutical composition for sustained release of an active agent as described herein, or a pharmaceutically acceptable acid addition salt thereof, the method comprising: (a) preparing a first phase, the first phase comprising a solvent, the active agent and a polymer; (b) preparing a second phase comprising a solvent; (c) passing the first phase and the second phase through a packed bed apparatus under laminar flow conditions, wherein the method results in the formation of microparticles; and (d) collecting the microparticles containing the active agent.

In certain aspects, the invention provides a method for making microparticles for use in a pharmaceutical composition for sustained release of an active agent as described herein or a pharmaceutically acceptable acid addition salt thereof, the method comprising: (a) preparing a first phase, the first phase comprising a solvent and the active agent; (b) preparing a second phase comprising a solvent and a polymer; (c) preparing a third phase containing a solvent; (d) combining the first phase and the second phase to create an emulsion; (e) passing the emulsion through a packed bed apparatus under laminar flow conditions with the third phase, wherein the method results in the formation of microparticles; and (f) collecting the microparticles containing the active agent.

In certain embodiments, the active agent is JPE1375, which has the following structure: Hoo-Phe-Orn-Pro-hle-Pff-Phe-NH2, the solvents are benzyl alcohol, and ethyl acetate, and the polymer is PLGA, with a mole ratio of lactide:glacolide 85:15.

In certain aspects, the invention provides a method for treating or preventing a disease or condition associated with complement activation, or a disease or condition where inhibition of the complement system leads to a relief of symptoms, the method comprising administering to a subject a sustained release pharmaceutical composition comprising an active agent as described herein and a PLGA polymer. In certain embodiments, the composition is administered by any of the methods described herein, or any other suitable method. In certain embodiments, the composition is administered to a subject who is in need of treatment.

In certain embodiments, the composition continues to release and/or maintain a therapeutic dose of the active agent for a period of about 60, 70, 80, 90, 100, 110, 120, 130, 140, 150, 160, 170, 180, 190, 200, 210, 220, 230, 240, or 250 days.

In certain embodiments, administering includes administering an effective dose of the composition of the invention, which composition continues to release and/or maintain therapeutic dose of the active agent for a period greater than 10, 20, 30, 40, 50, 60, 70, 80, 90, 100, 110, 120, 130, 140, 150, 160, 170, 180, 190, 200, 210, 220, 230, or 240 days to about 250 days.

In certain aspects, the disease or condition to be treated or prevented is selected from the group consisting of autoimmune diseases, acute inflammatory diseases, trauma, local inflammations, shock, burn, organ damage of a transplanted organ, organ damage of an organ to be transplanted, and transplant rejection, or any combination thereof.

In certain aspects, the disease or condition is selected from the group consisting of rheumatoid arthritis, ankylosis spodylitis, sarcoidosis, systemic lupus erythematosus, multiple sclerosis, psoriasis, septic shock, haemorrhagic shock, systemic inflammatory response syndrome (SIRS), multiple organ failure (MOF), asthma, vasculitis, myocarditis, dermatomyositis, inflammatory bowel disease (IBD), pemphigus, myasthenia gravis, glomerulonephritis, acute respiratory insufficiency, stroke, myocardial infarction, reperfusion injury, neurocognitive dysfunction, anti-phospholipid syndrome, burn, inflammatory diseases of the eye, local manifestations of systemic diseases, inflammatory diseases of the vasculature, and acute injuries of the central nervous system, or any combination thereof.

In certain aspects, the inflammatory disease of the vasculature is selected from the group comprising vasculitis, vascular leakage, and atherosclerosis.

In certain aspects, the inflammatory disease of the eye is selected from the group consisting of uveitis, age-related macular degeneration, diabetic retinopathy, diabetic macular edema, ocular pemphigoid, keratoconjunctivitis, Stevens-Johnson syndrome, and Graves ophthalmopathy, or any combination thereof.

In certain aspects, the condition is a local manifestation of a systemic disease, whereby the systemic disease is selected from the group comprising rheumatoid arthritis, SLE, type I diabetes, and type II diabetes.

In certain aspects, the manifestations are selected from manifestations occurring at or in the eye, the brain, the vessels, the heart, the lungs, the kidneys, the liver, the gastro-intestinal tract, the spleen, the skin, the skeletal system, the lymphatic system, the blood, or any combination thereof.

### BRIEF DESCRIPTION OF THE DRAWINGS

FIG. 1 shows in vitro real time release of JPE1375 PLGA sustained release formulation. Extended release from JPE1375 microparticle formulations were determined in PBS (phosphate buffered saline pH 7.4, 0.05% Tween 20, and 0.05% sodium azide). Microparticle formulations (15 mg±1 mg) were weighed into a 1.5 mL polypropylene centrifuge tube to which 1 mL of PBS was added. The tube was then placed in a 37° C. environmental chamber on a rotating mixer. The supernatant was sampled by centrifuging at 2000 rpm, removing 1 mL, and replacing with fresh PBS. The release media was assayed for JPE1375 by RP-HPLC.

FIG. 2 shows JPE1375 PLGA formulation payload in vitreous humor versus time. The total concentration of JPE1375 in vitreous humor was determined by a qualified Liquid Chromatography-Mass Spectrometry (LC-MS) assay. Animals were euthanized and vitreous humor was collected from excised eyes at the specified time points. Total JPE1375 concentration reported is the sum of free JPE1375 in solution plus JPE1375 contained in the microparticle formulation. The concentration of free JPE1375 was assumed to be much less than JPE1375 contained in the microparticle formulation, thus total JPE1375 concentration was used as a surrogate for JPE1375 contained in the microparticle formulation. FIG. 2 reports an estimate of the payload for microparticle formulation at the reported time points.

FIG. 3 shows in vivo retina tissue concentrations of JPE1375 active after a single intravitreous administration of JPE1375 PLGA microparticle formulations. Animals were euthanized and retina tissue was collected by microdissection at the specified time points. The concentration of JPE1375 in the retina tissue was determined by a qualified Liquid Chromatography-Mass Spectrometry (LC-MS) assay specific for JPE1375. Data are reported on a logarithmic scale with standard deviation error bars.

### DETAILED DESCRIPTION

A common problem addressed during development of therapeutic agents designed to treat chronic or slowly developing disease is a mechanism to deliver drug over a extended duration (days-months) to provide long term treatment with minimal dosing. Numerous strategies have been developed to address this problem including microparticle formulations, delivery devices, implants, pumps etc. Table 1 in Appendix A lists several products and highlights that the ability to deliver small molecule and peptide based therapeutic agents for periods greater than 1-2 months via a single dose is possible but not common. Table 1 also demonstrates that there are no formulations for sustained release of peptide therapeutic agents that deliver the therapeutic peptide for a period greater than 4 months.

To address this problem, the invention provides sustained release formulations which comprise as an active agent a complement system antagonist. The invention also provides a method for treatment of chronic or slow developing etiologies, for example but not limited to complement-mediated diseases, that benefit from persistent treatment with a therapeutic agent with clearance properties that render the therapeutic agent suboptimal for long term use.

The present invention relates to the composition and methods of use of a sterile, biocompatible, biodegradable, injectable, sustained release formulation of a complement C5aR antagonist with an effective treatment period of 2 months or greater, for example but not limited 4.5 months, 5 months, 6 months, 7 months, or 8 months, from a single administration. A key feature of the formulations is the ability to inject the formulation through a small bore needle (27 gauge or less). This feature allows administration of said formulations without employing invasive surgical procedures associated with implant and device based strategies. The formulation is designed to have minimal burst release (≦10%), a duration of release targeted to be 1 month or greater, 2 months or greater, 3-6 months, 4.5-6 month or longer to provide therapeutic doses for treatment or prevention of complement mediated diseases with 4 or less treatments per year.

The present invention relates to microencapsulation of small molecule or peptidic based antagonists of C5aR and their use in treatment of complement mediated diseases. The formulations have a reduced burst release and provided sustained release of C5aR antagonists in an injectable depot formulation suitable for local, systemic, treatment of complement mediated disease.

### DEFINITIONS

The terms “comprises,” “comprising,” “containing,” “having,” and the like, have the meaning ascribed to them in U.S. Patent law and mean “includes,” “including,” and the like.

As used herein the term “about” is used herein to mean approximately, roughly, around, or in the region of. When the term “about” is used in conjunction with a numerical range, unless specifically stated otherwise, it modifies that range by extending the boundaries above and below the numerical values set forth. In general, the term “about” is used herein to modify a numerical value above and below the stated value by a variance of 20 percent up or down (higher or lower).

The term “microparticle” refers to a particle or particles comprising the active agent of the invention, which particles have diameter of about 1 micron to about 1000 microns. Microparticles of the invention include without limitation microspheres, microcapsules, microsponges, microgranules and particles in general, with an internal structure comprising a matrix of an active agent and biodegradable polymer.

The term “biodegradable polymer” or “bioerodible polymer,” as used herein, refers to a polymer that dissolves or degrades within a period that is acceptable in the desired application once exposed to a physiological solution of pH ranging from about 6 to about 9 and at a temperature of ranging from about 25 C to about 38 C. The terms “biodegradable,” “bioerodible,” or “biocompatible” are used interchangeably. The terms polymer and matrix are used interchangeably.

The terms “ocular” and “ophthalmic” are used interchangeably.

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the subretinal space, the conjunctiva, the subconjunctival space, the episcleral space, the intracorneal space, the epicorneal space, the sclera, the pars plana, surgically-induced avascular regions, the macula, and the retina.

As used herein, an “ocular condition or indication” is a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, retinal pigmented epithelium, Bruch's membrane, optic nerve (i.e. the optic disc), and blood vessels and nerves which vascularize or innervate a posterior ocular region or site.

Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

Therapeutic/Active Agents

The term “active agent” is used interchangeably with the term “therapeutic agent or component.” In certain embodiments, the invention contemplates microparticles, formulations and uses thereof of active agents which are peptidomimetic C5a receptors antagonists of general structure as described in U.S. Ser. No. 10/564,788, U.S. Ser. No. 11/814,050, U.S. Ser. No. 11/915,892, the full contents of which are incorporated herein. Specific active agents for use in the inventive microparticles and formulations are disclosed in U.S. Ser. No. 10/564,788, U.S. Ser. No. 11/814,050, U.S. Ser. No. 11/915,892, and Schnatbaum et al., Bioorganic and Medicinal Chemistry Letters (2006) 5088-5092. The invention contemplates active agents as described in U.S. Ser. No. 10/564,788, U.S. Ser. No. 11/814,050, U.S. Ser. No. 11/915,892, Schnatbaum et al., or pharmaceutically acceptable acid addition salts thereof. Appendices B, C, D, and E provides non-limiting examples of the active agents contemplated for use in the invention.

In certain embodiments, the active agent is any one of the following peptidomimetic compounds: Hoo-Phe-Orn-Pro-hle-Pff-Phe-NH2, Ac-Phe-Orn-Pro-hle-Pff-Phe-NH2, Ac-Phe-Orn-Pro-hle-Mcf-Phe-NH2, Ac-Phe-Orn-Pro-hle-Dcf-Phe-NH2, Ac-Phe-Orn-Pro-hle-Bta-Phe-NH2, Ac-Phe-Orn-Pro-hle-Trp-Phe-NH2, Ac-Phe-Orn-Pro-cha-Trp-Phe-NH2, Ac-Phe-Orn-Pro-cha-Trp-Nle-NH2, Ac-Phe-Orn-Pro-cha-Trp-Arg-NH2, Ac-Phe-Orn-Pro-cha-Trp-Phe-OH, Ac-Phe-[Orn-Pro-cha-Trp-Arg], Ac-Ala-[Orn-Pro-cha-Trp-Arg], Ac-Phe-[Orn-Ala-cha-Trp-Arg], Ac-Phe-[Orn-Pro-ala-Trp-Arg], Ac-Phe-[Orn-Pro-cha-Ala-Arg], Ac-Phe-[Orn-Pro-cha-Trp-Ala], Ac-Phe-[Orn-Pro-cha-Trp-Har], Ac-Phe-[Orn-Pro-cha-Trp-Lys], Ac-Phe-[Orn-Pro-cha-Trp-Orn], Ac-Phe-[Orn-Pro-cha-Trp-Cit], Ac-Phe-[Orn-Pro-cha-Trp-Arg(NO₂)], Ac-Phe-[Orn-Pro-cha-Trp-Nva], Ac-Phe-[Orn-Pro-cha-Trp-Nle], Ac-Phe-[Orn-Pro-cha-Trp-Ile], Ac-Phe-[Orn-Pro-cha-Trp-Leu], Ac-Phe-[Orn-Pro-cha-Trp-Hle], Ac-Phe-[Orn-Pro-cha-Trp-Chg], Ac-Phe-[Orn-Pro-cha-Trp-Cha], Ac-Phe-[Orn-Pro-cha-Trp-Hch], Ac-Phe-[Orn-Pro-cha-Trp-Ocg], Ac-Phe-[Orn-Pro-cha-Trp-nle], Ac-Phe-[Orn-Pro-cha-Trp-Eag], Ac-Phe-[Orn-Pro-cha-Trp-Phe], Ac-Phe-[Orn-Pro-cha-Trp-Thi], Ac-Phe-[Orn-Pro-cha-Trp-1Ni], or Ac-Phe-[Orn-Pro-cha-Trp-2Ni], or any combination thereof.

In a certain embodiment the active agent is Hoo-Phe-Orn-Pro-hle-Pff-Phe-NH2, also referred to as JPE1375.

For non-proteinogenic amino acids a 3-letter code was used where the first letter indicates the stereochemistry of the C-alpha-atom, as described in Table 3 of U.S. Ser. No. 10/564,788, and Schnatbaum et al. A capital first letter stands for the L-form, a lower case first letter stands for the D-form of the correspondent amino acid, as described in Table 3 of U.S. Ser. No. 10/564,788, and Schnatbaum et al., the contents of which are fully incorporated herein. For proteinogenic amino acids the standard 3-letter codes were used. The following abbreviations are used non-proteinogenic: 1Ni 1-Naphthylalanine 2Ni 2-Naphthylalanine 3PP 3-Phenylpropionyl 5Ff Pentafluorophenylalanine 6FW 6-Fluoro-DL-tryptophane Aic 2-Aminoindan-2-carboxylic acid Amf Alpha-methyl-phenylalanine Aoa Aminooxyacetic acid Aoc 1-Aza-bicyclo-[3.3.0]-octan-2-carboxylic acid Aze Azetidine-2-carboxylic acid Bal beta-alanine Bhf beta-homophenylalanine Bta Benzothienylalanine Bzl Benzyl Cha beta-cyclohexylalanine Chg Cyclohexylglycine Chy cis-Hydroxyproline Cit Citrulline Ctb Cys(tBu) Dab 2,4-Diaminobutyric acid Dap 2,3-Diaminopropionic acid Def N,N-diethyl-phenylalanine Dff Phe(3,4-F) Eaa Phe(3,4-C1) Eaf Allylglycine Eag 2-Propargylglycine Eap Phe(4-tBu) Eay (2S,4S)-4-Phenyl-pyrrolidine-2-carboxylic acid Ebd Cys(Et) Ebo Cys(4-picolyl) Ebu Cys(3-picolyl) Ebw 3,3-Diphenylalanine Eby (S)-3-Amino-3-phenylpropanic acid Ecf Cys(O-3-picolyl) Ecg Cys(2-picolyl) Ecp His(tau-4-Methoxybenzyl) Ecr His(tau-methyl) Edn Cys(CH.sub.2-CH.sub.2-4-Pyridyl) Eec Cys(1-Methylene-1H-benzotriazole) Eep Cys(O2-Acm); 3-(acetylamino-methanesulfonyl)-2-amino-propionic acid Eew Arg(NO.sub.2) Egc DL-Trp(5-Me) Egy Phe(2,4-C1) Egz Phe(3-NO.sub.2) Eth Ethyl FAc F—CH.sub.2-CO-Fai-CONH.sub.2 Faz 3-Phenylpropionyl Fbi 2-(4-Pyridyl)acetyl Fbn Nicotinoyl Fbo Morpholine-4-carbonyl Fbp N,N-dimethyl-phenylalanine Fci Piperidine-3-carbonyl Fck HO—CH.sub.2-(CHOH).sub.4-C.dbd.N—O—CH.sub.2-CO—Fcn norArg(CH.sub.2CH.sub.2); 2-Amino-4-(4,5-dihydro-1H-imidazol-2-ylamino)-butyric acid Fco bisnorArg(CH.sub.2CH.sub.2); 2-Amino-3-(4,5-dihydro-1H-imidazol-2-ylamino)-propionic acid Fcp 2-Amino-5-[bis-(4,5-dihydro-1H-imidazol-2-yl)-amino]-pentanoic acid Ffa Arg(CH.sub.2CH.sub.2); 2-Amino-5-(4,5-dihydro-1H-imidazol-2-ylamino)-pentanoic acid Fha 2-Morpholin-4-yl-acetyl Fhb N-(2,3-Dihydroxy-propyl)-formamidyl Fhi 2-[2-(2-Methoxy-ethoxy)-ethoxy]-acetyl Fhu-C(NH)—NH.sub.2 Fib Arg(4× Me), [(4-amino-4-carboxy-butylamino)-dimethylamino-methylene]-dimethyl-ammonium Fid Methoxyoxalyl G23 Orn(SO.sub.2Me) G24 N-(n-Propyl)-glycine G25 N—(CH.sub.2CH.sub.2OCH.sub.3)-glycine G26 N—(CH.sub.2Furyl)-glycine G27 N—(CH.sub.2Pyridyl)-glycine G30 N—(CH.sub.2CH.sub.2CH.sub.2(2-oxo-pyrrolidine-1-yl))-glycine G31 N—(CH.sub.2CH.sub.2(3,4-dimethoxyphenyl))-glycine Guf Phe(4-guanidine) Har homo-arginine Hch homo-cyclohexylalanine Hci homo-citrulline Hle homo-leucine Hoo Hydroorotic acid; (S)-2,6-dioxo-hexahydro-pyrimidine-4-carbonyl Hse homo-Serine HyA Hyp(Ac) Hym Hyp(Me) Hyp trans-hydroxyproline L19 1-(Methoxymethyl)-2-phenyl-ethylamino L22 norArg Mcf Phe(3-C1) Mff Phe(3-F) Mmf Phe(3-Me) Mmy Phe(3-OMe) Mpa 3-(3-Pyridyl)-alanine Nip Nipecotic acid Nle Norleucine NMA N-Me-alanine NMD N-1-Me-asparagine NMF N-Me-phenylalanine NMS N-Me-serine Nva Norvaline Ocf Phe(2-C1) Off Phe(2-F) Ohf (S)-2-Hydroxy-3-phenyl-propionyl Oic Octahydroindole-2-carboxylic acid Omf Phe(2-Me) Opa 3-(2-Pyridyl)-alanine OrA Orn(Ac) OrE Orn(Et.sub.2); 2-Amino-5-diethylamino-pentanoic acid Orn Ornithine Otf Phe(2-CF3) Paf Phe(4-NH.sub.2) Pcf Phe(4-C1) Pff Phe(4-F) Phg Phenylglycine Pip Pipecolinic acid Pmf Phe(4-Me) Ppa 3-(4-Pyridyl)-alanine Tff Phe(3,4,5-F) Thi 2-Thienylalanine Tic 1,2,3,4-Tetrahydroisochinoline-3-carboxylic acid Tiq Tetrahydroisochinoline-1-carbonxylic acid Tmg .dbd.C(NMe.sub.2)-NMe.sub.2 XX1 2-Amino-3-(4-piperidinyl)propionic acid XX2 4-Guanidyl-piperidinyl-alanine

Biodegradable Polymers

A variety of biodegradable polymers used for controlled release formulations are well known in the art and are contemplated for use in the sustained formulations of the invention. Suitable polymers for example include, but are not limited to, poly(hydroxy acids), tyrosine or “pseudo”-poly(amino acids) based polymers, poly(lactic acid), poly(glycolic acid), poly(lactic acid-co-glycolic acid), polycaprolactones, polyanhydrides, polycarbonates, polyamides, polyesters, polyorthoesters, polyhydroxybutryate, certain types of protein and polysaccharide polymers, and blends, copolymers or mixtures thereof. Suitable polymers for use in sustained release formulations are known in the art.

The biodegradable polymers are optionally capped or un-capped. Capped polymers include, but are not limited to, those having esterified or amidated end groups. Un-capped polymers include free hydroxyl or carboxyl end-groups. In one embodiment, the microparticles comprise free-acid poly (lactic acid-co-glycolic acid). In another embodiment, the microparticles comprises lauryl or N-capped poly(lactic acid-co-glycolic acid).

In certain embodiment, the polymer is poly (hydroxy acids). In one embodiment the polymer is poly (lactic acid-co-glycolic acid) (“PLGA”) that degrades by hydrolysis following exposure to the aqueous environment of the body. The polymer is then hydrolyzed to yield lactic and glycolic acid monomers, which are normal byproducts of cellular metabolism. The rate of polymer disintegration can vary from several weeks to several months, to periods of greater than one year, depending on several factors including polymer molecular weight, ratio of lactide to glycolide monomers in the polymer chain, and stereoregularity of the monomer subunits (mixtures of L and D stereoisomers disrupt the polymer crystallinity enhancing polymer breakdown). Microparticles may contain blends of two and more biodegradable polymers, of different molecular weight and/or monomer ratio.

PLGA may have any suitable monomer ratio of lactide:glycolide. The particular ratio of the polymers may be determined based on pharmacokinetic evaluations. In one embodiment the amount of lactide ranges from 0-100%. In another embodiment, the lactide ranges from 70-100%, 76-100%, 80-100%, 80-95%. In another embodiment, the lactide ranges from 76-95%, 85-95%. In another embodiment, the lactide ranges from 85-90%. In another embodiment, the lactide ranges from 90-95%. In certain embodiments, PLGA polymer has mole ratio of lactide:glycolide of 100:0, 92:8, 86:14; 85:15, 76:24, 65:35, or 50:50. In one embodiment, the ratio of lactide:glycolide is 85:15.

The inherent viscosity of the polymers can be chosen so that it is suitable for using the polymers in the inventive microparticles and formulations. In one embodiment, the inherent viscosity of the biodegradable polymer may be in the range 0.1 to 2.0 dL/g. In another embodiment, the inherent viscosity of the biodegradable polymer ranges from about 0.1 to about 1.0 dL/g, about 0.3 to about 0.7 dL/g. In another embodiment, the inherent viscosity of the biodegradable polymer is about 0.16 dL/g, 0.35 dL/g, 0.39 dl/g, 0.4 dL/g, 0.41 dL/g, 0.52 dL/g, 0.66 dL/g or 0.61 dL/g.

Other features of the polymers suitable for use in the invention include but are not limited to any one of the following features or a combination thereof: the lactic or glycolic acid polymer block size, residual monomer, metal catalyst, or polymer polydispersity.

A surfactant is optionally used in order to provide formulations that have the required syringability. In one embodiment, a surfactant is used for providing a stable emulsion during the process of forming the microparticles of the present invention. In another embodiment, a surfactant is used for preventing agglomeration during lyophilization during the process of forming the microparticles. In another embodiment, a surfactant is used for preventing agglomeration within the injection vehicle during the process of delivering the microparticles. Without wishing to be bound by theory, surfactants may provide batch-to-batch consistency of microparticles by forming a thin layer of material around the microparticles that helps prevent clumping. Any suitable surfactant may be used. Suitable surfactants include, but are not limited to, cationic, anionic, and nonionic compounds such as poly(vinyl alcohol), carboxymethyl cellulose, lecithin, gelatin, poly(vinyl pyrrolidone), polyoxyethylenesorbitan fatty acid ester (Tween 80, Tween 60, Tween 20), sodium dodecyl sulfate (SDS), mannitol and the like.

The morphology, shape, size, drug content or any other characteristics of the microparticles may be determined by any suitable method known in the art. For example, microparticle morphology may be examined by Electron Scanning Microscopy. Drug/active agent content and/or stability in the microparticles may be determined by HPLC, or LC-MS bioanalytical assay, using any suitable protocol.

Suspendability and syringibility, for example, in various diluents or vehicles suitable for delivery and administration of the inventive formulations, may be determined by any known method. The following is a non-limiting list of vehicles which can be used to suspend the microparticles and formulations of the invention: Saline; PBS; PBS with 0.05 or 0.5% SDS; PBS with 0.02% Tween20; PBS with 1% Mannitol; PBS with 0.5% SDS and 0.5% CMC; PBS with 0.02% Tween20 and 0.5% CMC; PBS or water with 5% mannitol, 0.5% CMC and 0.05% Tween20.

In vitro and in vivo release of the active agent from the microparticles and formulation of the invention can be measured by any suitable method known and practiced in the art. Non-limiting examples of such methods include HPLC, LC-MS, or any other methods.

In certain embodiments, the microparticles and formulations of the invention are prepared in a sterile form, by any suitable methods, for example by gamma irradiation.

Mircroparticles: Composition (% Active), Size, Morphology

The amount of the active agent included in the inventive formulations may vary depending on the effective therapeutic dose required and on the desired rate of release from the sustained release compositions. In certain embodiments, the active agent can be at least 5%, at least 10%, at least 15%, at least 20%, at least 25%, but no more than 80%, 90% of the microparticle weight. In other embodiments, the active agent can be about 5%, about 6%, about 7%, about 8%, about 9%, about 10%, about 11%, about 12%, about 13%, about 14%, about 15%, about 16%, about 17%, about 18%, about 19%, about 20%, about 25% of the weight of the microparticle. In other embodiments, the active agent is 5-15% of the weight of the microparticle, is 5-10%, 5-11%, 5-12%. 5-13%, 5-14% of the weight of the microparticle, is 10-15% of the weight of the microparticle, is 7-10%, 7-12%, 10-12% of the weight of the microparticle. In other embodiments the active agent is 6, 8, 10, 12, 14, 16% of the weight of the microparticle. In other embodiments, the sustained release composition comprises a minimum of 3, 5, 5-8, 6-8, >8 to about 10, 11, 12, 13, 14, 15, 16, 17, 18, 19, 20 weight % of peptide or peptidomimetic C5aR antagonist to the complement cascade. In other embodiments, the active agent comprises 1 to 90 wt. % of the microparticle, comprises about 2 to 20 wt. % of the microparticle, comprises about 2 to 20 wt. % of the microparticle.

The microparticles of the invention can be of any suitable size. In certain embodiments, the size of the microparticles is uniform. The size of the microparticles can vary from about 10 microns to about 1000 microns, 1 to 100 microns. In certain embodiments, the size of the microparticles is from 25 microns to 63 microns, 20 microns to 63 microns, 25 microns to 45 microns, 20 microns to 45 microns, 25 to 65 microns. In certain embodiments, the majority of the microparticles in a formulation have size of 25 microns to 63 microns. 20 microns to 63 microns, 25 microns to 45 microns, 20 microns to 45 microns.

In certain embodiments and depending on the method of administration, the size of the microparticles is such that the microparticles can readily be delivered, for example but not limited through a suitable size syringe. In a non-limiting example of administering the microparticles of the invention, the microparticles readily pass through a needle of 25 gauge, 27 gauge, 29 gauge, 30 gauge.

In certain embodiments, the microparticles of the invention have density suitable for preparation of the inventive formulations.

Methods for Making Sustained Release Microparticles/Formulations of the Invention

Microparticles for controlled release compositions of the present invention may be made by any emulsion process known in the art. Non-limiting examples include methods using standard oil/water, discontinuous/continuous phase. In general, the methods have a first phase consisting of or comprising an organic solvent, a polymer and an active agent dissolved or dispersed in the first solvent or mixture of solvents. This is sometimes referred to as the discontinuous phase. The second phase is immiscible with the first phase, is often aqueous based and optionally contains excipients, an emulsion stabilizer and/or solvents from the first phase. The second phase is sometimes referred to as the continuous phase. The first and the second phases are emulsified and, after an emulsion is formed, the first solvent(s) is removed from the emulsion, producing hardened microparticles. An alternative method involves the formation of a “double emulsion”. In this method, a first phase, often called an “internal phase”, is produced and normally aqueous based containing an active agent, and optionally excipients such as a stabilizer(s). In a non-limiting example, the water phase may include 1% PVA, 2% benzyl alcohol, 4.35% Ethylacetate. In a non-limiting example, the water phase may include 0.8% PVA, 3.5% benzyl alcohol, 0.0% Ethylacetate in pH7.0 phosphate buffer. A second-phase normally consists of an organic solvent or solvent blends and a polymer. The first and second phases are emulsified to form a water-in-oil “internal emulsion”. A third-phase usually aqueous based optionally containing excipients, a surfactant(s) and/or, the second solvent(s). The internal emulsion is then emulsified again with the third phase to form an oil-in-water “external emulsion”. After the external emulsion is formed, the organic solvent is removed from the emulsion, producing hardened microparticles. Emulsions may be formed by a variety of techniques, such as affecting mixing of phases, adding energy to the phases or increasing interaction between phases. One such technique is the use of a batch device for mixing the first and second phases under turbulent conditions such as with a stirrer. Other batch processes may employ a homogenizer or a sonicator. In another technique, an emulsion is formed by continuously mixing the first phase and second phase, in-line, using turbulent flow conditions, as in the use of an in-line dynamic mixer or an in-line static mixer. Efficient removal of the first or discontinuous phase solvent(s) from the second or continuous phase is required to prepare particles containing low residual levels of solvent(s). This is desired to reduce exposure to these agents. Removal can be effected by evaporation, extraction or a combination of the two. Variables to be considered to affect removal include but are not limited to time, agitation, extraction procedure, volume, temperature, local atmospheric composition, pressure, and volume.

Non-limiting examples of emulsion processes suitable for making the microparticles and sustained release formulations of the invention are disclosed in WO 2005/003180, U.S. Ser. No. 10/553,003, or U.S. Ser. No. 11/799,700, the full contents of which applications are incorporated herein.

In one embodiment, a method for making the sustained release microparticles of the invention comprises: (a) emulsifying oil/water phase, wherein the oil phase comprises the active agent as described herein and the biodegradable polymer; (b) extracting the solvent; (c), optionally carrying out a diafiltration and (d) collecting the microparticles containing the active agent and the biodegradable polymer. Steps (a) and (b) can be carried out simultaneously or sequentially. In another embodiment, a method for making the sustained release microparticles of the invention comprises: (a) emulsifying oil/water phase, wherein the oil phase comprises the active agent as described herein and the biodegradable polymer, and wherein the water phase pH is controlled, near the isoelectric point of the active agent, in a non-limiting example the pH range is pH 6.0 to 8.0; (b) extracting the solvent, wherein the extraction buffer is near the isoelectric point of the active agent, pH 6.0-8.0; (c) optionally carrying out a diafiltration; and (d) collecting the microparticles containing the active agent and the biodegradable polymer. Steps (a) and (b) can be carried out simultaneously or sequentially. In certain embodiments, extraction and diafiltration are carried out concurrently. In other embodiments, extraction and diafiltration are carried out sequentially. In certain embodiments, the methods produce microspheres. In certain embodiments, the methods include a step of drying and/or lyophilizing the microparticles.

In certain embodiments a pharmaceutical composition of the invention is prepared by a method comprising: (a) preparing a first phase, the first phase comprising a solvent, the active agent and a polymer; (b) preparing a second phase comprising a solvent; (c) passing the first phase and the second phase through a packed bed apparatus under laminar flow conditions, wherein the method results in the formation of microparticles; and (d) collecting the microparticles containing the active agent.

In certain embodiments a pharmaceutical composition of the invention is prepared by a method comprising: (a) preparing a first phase, the first phase comprising a solvent and the active agent; (b) preparing a second phase comprising a solvent and a polymer; (c) preparing a third phase containing a solvent; (d) combining the first phase and the second phase to create an emulsion; (e) passing the emulsion through a packed bed apparatus under laminar flow conditions with the third phase, wherein the method results in the formation of microparticles; and (f) collecting the microparticles containing the active agent.

In certain embodiments, non-limiting examples of solvents which can be used in the methods of the invention include methylene chloride, ethyl acetate, benzyl alcohol, DMSO, methanol, acetone, acetic acid, propylene carbonate or any other suitable solvent, for example but not limited to a solvent in which the biodegradable polymer is soluble. In certain embodiments, microparticles are made using dichloromethane and benzyl alcohol. See Table 2 in Appendix A. In other embodiments, microparticles are made using ethyl acetate and benzyl alcohol. See Table 3 in Appendix A.

In certain embodiments the methods comprise an extraction step, and use a solvent extraction buffer. In certain embodiments, the solvent extraction buffer used in the methods for making the inventive sustained release formulations has neutral pH. In certain embodiments, the neutral pH is controlled. In certain embodiments the pH is pH 7.0.

In other embodiments, the water phase has an emulsifying agent, for example but not limited to poly(vinyl) alcohol (PVA). Other suitable emulsifying agents are also contemplated. In other embodiments, the water phase has a neutral pH. In certain embodiments, the neutral pH is controlled. In certain embodiments the pH is pH 7.0. Without wishing to be bound by theory, controlling the pH during the methods for making microparticles, for example maintaining neutral pH 7.0 for the extraction buffer, leads to consistently reproducible microparticle size, and drug content, which is readily controlled.

In other embodiments, the methods for making the inventive sustained release formulation includes a step of diafiltration, following the emulsification and primary extraction steps.

Microparticle formulation preparation: JPE1375 microparticle formulations were prepared using a oil-in-water emulsion/solvent extraction procedure. Briefly JPE1375 (0.100 g) was dissolved in benzyl alcohol (1.0 mL). Poly(lactide-co-glycolide) (PLGA, 0.400 g) was dissolved in ethyl acetate and upon complete dissolution the JPE1375 solution was added to the PLGA solution to form the oil phase.

The aqueous phase was prepared by dissolving poly vinyl alcohol (PVA, 0.475 g) in water (46.4 mL). The mixture was heated to 80° C. for one hour with vigorous stirring to effect solution and then cool solution to ambient temperature. Benzyl alcohol (1.0 g) and ethyl acetate (2.175 g) are added to the PVA solution and stirred for at least 10 minutes to provide a homogeneous aqueous phase solution. An emulsion is prepared by combining the oil phase (0.7 mL/min) and aqueous phase (1.3 mL/min) in a packed back emulsifier, such as described in US Pub 20070207211 or US Pub 20070190154. The resulting emulsion (2.0 mL/min) is combined with the extraction medium (water, 50 mL/min) into an extraction vessel held at 22° C. The extraction medium is then heated from 22° C. to 50° C. over 60 minutes while concurrently diafiltering with water (8 diafiltration volumes) over approximately 80 minutes. The temperature of the extraction tank is held at 50° C. for 60 minutes after completion of the diafiltration step and then cooled to 25° C. The microparticles are collected between stacked 63 μm and 25 μm sieves using 0.1% Tween 20 to wash particles collected on the 63 μm sieve. The 63 μm sieve is removed the particles collected on the 25 μm sieve are washed three times with water. The microparticles are collected from the 25 μm sieve and lyophilized to provide the formulation.

JPE1375 microparticle formulations can also be prepared according to the method described above using 50 mM phosphate buffer (pH 7.0) containing 0.8% PVA and 3.5% benzyl alcohol as the aqueous phase. A 50 mM phosphate buffer (pH7.0) solution is used to replace water as the extraction medium. Microparticles are prepared, extracted, diafiltered against water, isolated washed and dried as described in the example above, prepared using a oil-in-water emulsion/solvent extraction procedure.

Delivery

The compositions of the invention may be delivered to the area of treatment by any suitable method known in the art. Non-limiting examples include parenteral administration, such as intravenous or intramuscular injection. Other alternative methods of administration may also be used, including but not limited to intradermal administration, pulmonary administration, buccal administration, transdermal and transmucosal administration. Transmucosal administration may include, but is not limited to, ophthalmic, vaginal, rectal and intranasal. Various such methods of administration are well known in the art.

In certain aspects, methods for treatment of local or localized inflammatory diseases or disorders are provided, wherein such diseases or disorders can be treated with local delivery of the therapeutic composition, while there is low systemic exposure to the therapeutic agent. For example, Tables 6-8, demonstrate that plasma levels of the active agents are below limit of quantitation (BLQ) after ocular delivery of the inventive formulation. In these methods, the treatment comprises local delivery of the inventive compositions as to achieve local or localized treatment of the disease or conditions, wherein local therapeutic levels of the agents are achieved and maintained with limited systemic exposure to the therapeutic agent. In a non-limiting example, when the compositions of the invention are used for ophthalmic indications, the compositions may be administered via any suitable method which delivers the sustained release composition of the invention to the eye, including but not limited to specific areas of the eye. In certain embodiments, the composition may be administered in the eye via a needle, or a catheter. In a non-limiting example, the inventive compositions are delivered by a intravitreous injection using a 27 gauge needle. In another non-limiting example, the compositions of the invention may be delivered locally, for example by injection to a diseased joint, for treatment of an inflammatory disease such as but not limited to rheumatoid arthritis.

Dosage

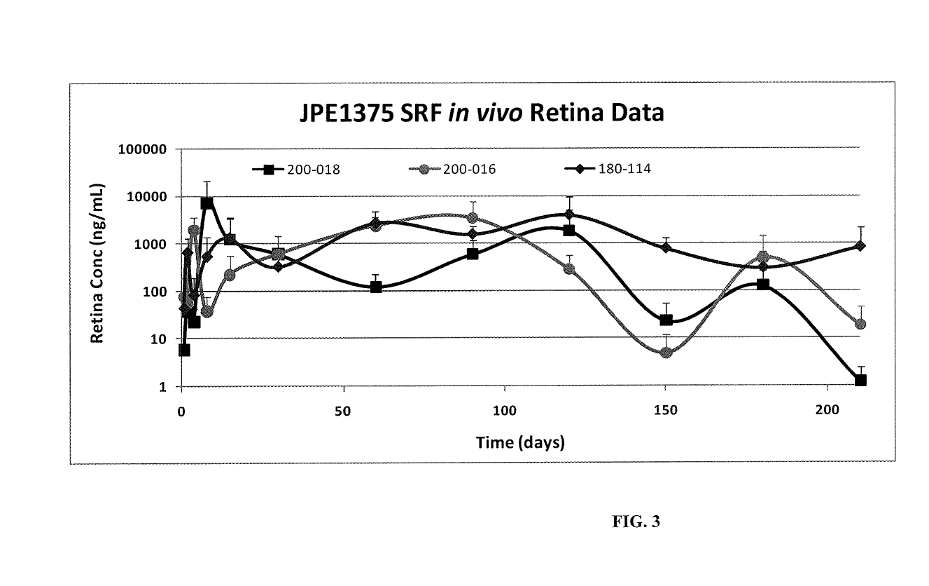

The formulation of the invention can be released at a controlled rate when the formulation is placed in the eye. Such rates may range from about 0.003 micrograms/day to about 5000 micrograms/day, be 0.0 to 50 micrograms/day, 0.0 to 5 micrograms/day, 1.0 to 5 micrograms/day. In certain embodiments, the release is zero because the formulations are known to have variable release kinetics. It is possible and likely that in certain embodiments, in a non-limiting example at about 6 month after delivering the formulation, there is a period where no active agent is released but the minimum therapeutic level is still maintained if the target tissue concentrations and active agent molecule PK are appropriately matched. This is illustrated by the retina data included in the instant Examples. For example, retina concentrations for all formulations listed in FIG. 3 drop very rapidly after dosing but therapeutic levels are quickly established and maintained subsequently.

Thus doses range 0-50 micrograms/day, and at a rate to maintain a therapeutic level such as approximately ≧60 ng/mL for the duration of treatment. For posterior ophthalmic indications this range is 0-20 microg/day, 0-10 microg/day, or 0-5 microg/day. In certain embodiments, retina levels are approximately 60 nM or greater (63 ng/mL or 63 ng/g assuming tissue density is 1).

Various publications are referenced throughout this application. The full contents of these publications are incorporated by reference herein. The following non-limiting examples are included to demonstrate particular embodiments of the invention. Those of skill in the art should, in light of the present disclosure, appreciate that many changes can be made in the specific embodiments which are disclosed and still obtain a like or similar result without departing from the spirit and scope of the invention.

### EXAMPLES

### Example 1

### Methods Used to Manufacture Microparticles for Use in a Sustained Release Formulation Comprising JPE1375 and PLGA

Equipment/Materials—Raw Materials: JPE1375: Jerini Ophthalmic; Benzyl Alcohol: EMD, 1.00981; PLGA, 85/15 □L 4A: Lakeshore (lot 97-12-201); Ethyl Acetate: Sigma-Aldrich, 27227; Poly(vinyl alcohol): Spectrum, P1180; Water.

Equipment:—Emulsification: 2×10 ml glass vials for mixing oil phase parts; 2× peristaltic pumps for oil/water phases; ¼″ OD×0.18″ID EZE column packed with 350-400 μm borosilicate glass beads; Column contains 150 um screens at the ends.

Equipment—Solvent Extraction: 1-L agitated glass tank; Extraction/diafiltration supply pump; CFF circulation pump; CFF housing and filter element; Refrigerated circulating water bath.

Equipment—Sieving and Drying: 2×8″ SS Sieves—25 μm and 63 μm; 50 ml polypropylene centrifuge tube; Liquid nitrogen; A lyophilizer.

Procedure

Calibrate peristaltic pumps; Prepare extraction tank and CFF loop; Flush EZE by pumping 20 mL 50:50 (v/v) benzyl alcohol:ethyl acetate solution through oil phase line to waste tank at 5 mL/min and then pump oil line and EZE dry. Finish flush by pumping 25 mL water through water phase line to waste tank at 5 mL/min and then pump water line and EZE dry; Program temperature controller to heat up from 22° c. to 50° C. in 60 min; Start extraction tank jacket temperature controller at 22° C.

Oil/Water Phase and Extraction Media Preparation

Weigh 0.400 g PLGA into glass vial. Add 1.000 mL ethyl acetate into the same container. Seal the container and agitate (vortex) until dissolved. Weigh 0.100 g JPE1375 into a separate glass vial. Add 1.000 mL benzyl alcohol into the same container. Seal the container and agitate (vortex) for 5 seconds, and then set the container on the benchtop for at least 15 min. Add the JPE1375 solution to the PLGA solution and mix to produce oil phase solution. Prepare 0.5 L extraction water.

Prepare 50 g water phase: Dissolve 0.475 g PVA in 46.35 g of water by heating to approximately 80° C. for one hour while stirring vigorously. Allow the PVA/buffer solution to cool to room temperature. Add 1.0 g benzyl alcohol and 2.175 g of ethyl acetate to the PVA solution prepared in 6.2.6.1, and stir vigorously for at least 10 min.

Prepare 4 L water for diafiltration.

Emulsification and Primary Extraction

Prime oil line with oil phase and water line with water phase, up to the EZE inlet. Prime extraction line to tank inlet. Connect emulsion tube to dip-tube. Start agitation, approximately 150 rpm. Set extraction pump to deliver 0.5 L and start extraction flow at 50 mL/min. Two minutes after starting extraction pump, start water flow at 1.2-1.3 mL/min and then start oil flow at 0.7 mL/min. When oil phase runs out, move oil phase line into a 3.9% benzyl alcohol in water solution. When EZE outlet turns clear, increase both oil phase pump and water phase pump to 5 mL/min for 20 seconds, then stop both pumps. Observe completion of extraction buffer addition. Collect sample for CG analysis.

Diafiltration

Prepare for diafiltration: Connect diafiltration water supply tank and prime line to tank. Open bottom tank valve and start cross-flow filtration (CFF) circulation pump at 1.5 L/min. Note 0.5 L working volume level. Set diafiltration pump to deliver 4000 mL and start water flow at 25-50 mL/min. Pump filtrate from CFF at 50 mL/min to maintain constant tank level. Start temperature ramp from 22° C. to 50° C. over 60 min. Collect GC and HPLC samples as needed. Observe completion of the diafiltration step after 4000 mL have been delivered. Close bottom tank valve and stop CFF recirculation pump. Flush CFF loop with 300 mL water into tank. 60 minutes after end of water addition, decrease temperature setpoint of reactor jacket to 5° C.

Sieving and Drying

After suspension has cooled to 25° C. or below, drain suspension slowly thru bottom valve to 25 μm-63 μm hand sieves. Shake sieves and control suspension flow rate, as necessary. Rinse top 63 μm sieve with 0.1% Tween 20 and remove top sieve. Rinse bottom 25 μm sieve with water at least 3× and collect microspheres in a tared 50 mL polypropylene centrifuge tube. Cap tube. The total volume of microsphere suspension should be approximately 5 mL-15 mL for complete drying in the time noted below. Shell-freeze suspension in liquid nitrogen. Place microspheres on lyophilizer and let dry for 20-24 hours.

### Example 2

### Methods Used to Manufacture Microparticles for Use in Sustained Release Formulation Comprising JPE1375 and PLGA, with Control of pH

Equipment/Materials:

Raw Materials: JPE1375: Jerini Ophthalmic; Benzyl Alcohol: EMD, 1.00981; PLGA, 85/15 □L 4A: Lakeshore (lot 97-12-201); Ethyl Acetate: Sigma-Aldrich, 27227; Poly(vinyl alcohol): Spectrum, P1180; Sodium Phosphate, monobasic, monohydrate' Sodium Phosphate, dibasic, anhydrous; Water.

Equipment—Emulsification: 2×10 ml glass vials for mixing oil phase parts; 2× peristaltic pumps for oil/water phases; ¼″ EZE.

Equipment-Solvent Extraction: 1-L agitated glass tank; Extraction/diafiltration buffer supply pump; CFF circulation pump; CFF housing and filter element; Refrigerated circulating water bath.

Sieving and Drying: 2×8″ SS Sieves—25 μm and 63 μm; 50 ml polypropylene centrifuge tube; Liquid nitrogen; A lyophilizer.

Procedure

Setup: Calibrate peristaltic pumps. Prepare extraction tank and CFF loop. Flush EZE by pumping 20 mL 50:50 (v/v) benzyl alcohol:ethyl acetate solution through oil phase line to waste tank at 5 mL/min and then pump oil line and EZE dry. Finish flush by pumping 25 mL water through water phase line to waste tank at 5 mL/min and then pump water line and EZE dry. Program temperature controller to heat up from 22° c. to 50° C. in 60 min. Start extraction tank jacket temperature controller at 22° C.

Oil/Water Phase and Extraction Media Preparation: Weigh 0.8000 g PLGA into glass vial. Add 3.333 mL ethyl acetate into the same container. Seal the container and agitate (vortex) until dissolved. Weigh 0.2000 g JPE1375 into a separate glass vial. Add 3.333 mL benzyl alcohol into the same container. Seal the container and agitate (vortex) for 5 seconds, and then set the container on the benchtop for at least 15 min. Add the JPE1375 solution to the PLGA solution and mix.

Solvent Extraction Buffer: Prepare 1 L pH 7.0, 50 mM phosphate buffer for solvent extraction by dissolving 2.918 g sodium phosphate, monobasic, monohydrate and 4.096 g sodium phosphate, dibasic, anhydrous in water in a 1 L volumetric flask. Balance pH to 7.0 by slow addition of dilute sodium hydroxide solution or dilute phosphoric acid, and fill to 1 L with water.

Prepare 50 g water phase. Begin by preparing 100 mL pH 7.0, 50 mM phosphate buffer by dissolving 0.292 g sodium phosphate, monobasic, monohydrate and 0.410 g sodium phosphate, dibasic, anhydrous in water in a 100 mL volumetric flask. Balance pH to 7.0 by slow addition of dilute sodium hydroxide solution or dilute phosphoric acid, and fill to 100 mL with water. Dissolve 0.40 g PVA in 47.85 g of the 50 mM, pH 7.0 phosphate buffer prepared in 7.2.5.1 by heating to approximately 80° C. for one hour while stirring vigorously. Allow the PVA/buffer solution to cool to room temperature. Add 1.75 g benzyl alcohol to the PVA solution and stir vigorously for at least 10 min.

Prepare 4 L water for diafiltration.

Emulsification and Primary Extraction: Prime oil and water phase lines to EZE inlet. Prime extraction line to tank inlet. Connect emulsion tube to dip-tube. Start agitation. Set extraction pump to deliver 1 L and start extraction flow at 70 mL/min. Three minutes after starting extraction pump, start water flow at 1.4 mL/min and then start oil flow at 0.7 mL/min. When oil phase runs out, move oil phase line into water phase container. When EZE outlet turns clear, increase both oil phase pump and water phase pump to 5 ml/min for 15 seconds, then stop both pumps. Observe completion of extraction buffer addition. Collect sample for CG analysis.

Diafiltration: Connect diafiltration water supply tank and prime line to tank. Open bottom tank valve and start CFF circulation pump at 1.5 L/min. Concentrate suspension ×2 by removing 500 ml thru filtrate line into a 500 mL graduated cylinder. Set diafiltration pump to deliver 4 L and start water flow at 50 mL/min. Adjust filtrate flow rate to 50 mL/min to maintain constant tank level. Start temperature ramp from 22° c. to 50° c. over 60 min. Collect GC and HPLC samples as needed. Observe completion of the diafiltration step after 4 L have been delivered. Close bottom tank valve and stop CFF recirculation pump. Flush CFF loop with 300 mL water into tank. 60 minutes after end of water addition, decrease temperature setpoint of reactor jacket to 5° C.

Sieving and Drying: After suspension has cooled to 25° C. or below, drain suspension slowly thru bottom valve to 25 μm-63 μm hand sieves. Shake sieves and control suspension flow rate, as necessary. Rinse top 63 μm sieve with 0.1% Tween 20 and remove top sieve. Rinse bottom 25 μm sieve with water at least 3× and collect microspheres in a tared 50 ml polypropylene centrifuge tube. Cap tube. The total volume of microsphere suspension should be approximately 5 mL-15 mL for complete drying in the time indicated below. Shell-freeze suspension in liquid nitrogen. Place microspheres on lyophilizer and let dry for 20-24 hours.

### Example 3

### Characterization of Sustained Release Formulation Prepared by the Methods Described in Example 1 and Example 2

Reverse Phase HPLC method: A method was developed utilizing a linear acetonitrile/water gradient containing 0.05% trifluoroacetic acid with UV detection at 230 nm. The method is summarized in the Table 4 below:

| Column: | Phenomenex C-18 Luna, 30 × 2 mm, | | |
|---|---|---|---|
| | 3 μm, 100 Å | | |
| Injection Volume: | 50 μL | | |
| Flow Rate: | 0.4 mL/min | | |
| Mobile Phase: | A: 0.05% TFA in water | | |
| | B: 0.05% TFA in acetonitrile | | |
| | Time | % A | % B |
| Gradient: | 0.00 | 100 | 0 |
| | 1.00 | 100 | 0 |
| | 16.00 | 5 | 95 |
| | 21.00 | 5 | 95 |
| | 24.00 | 100 | 0 |
| | 30.00 | 100 | 0 |

| | |
|---|---|
| Column Temperature: | No column temperature was specified by |
| | Jerini. Temperature control at 25° C. |
| | was employed. |

Determination of JPE1375 Content in Microparticle Formulations:

The drug or peptide content was determined by an extraction/HPLC assay. Approximately 5 mg of microspheres were weighed into a 2 mL polypropylene tube to which an aliquot of 750 μL of 5/95 Dimethylsulfoxide/Acetonitrile (v/v) was added. The tube was vortex mixed for 5 seconds and sonicated for 5 minutes. To the resulting clear solution an aliquot of 1250 μL of 0.1% TFA (v/v) was added and the tube was vortex mixed for 5 seconds. The tube was centrifuged at 14000 RPM for 2 minutes and the supernatant was analyzed by HPLC.

Particle Size Analysis

Particle size was measured using a Coulter Particle Size Analyzer Optical Module LS 100Q with a small volume module.

Determination of Residual Solvents in Microparticle Formulations:

Approximately 20 mg of microspheres were accurately weighed into an auto sampler vial. An aliquot of 1 mL NMP was added to the vial and completely mixed. The injection volume was 1 μL on a Restek Stabilwax, Crossbond Carbowax-PEG column, 30 M, 0.32 mm ID column (part#10654). The oven temperature was held at 40° C. for 1 minute then ramped up to 220° C. at 6° C. per minute. Peak areas from the sample were compared to benzyl alcohol and ethyl acetate standard calibration curves with concentrations ranging from 0.2088 mg/mL to 0.0261 mg/mL for benzyl alcohol and 0.3588 mg/mL to 0.04485 mg/mL for ethyl acetate. This method allows for quantitation of residual ethyl acetate and benzyl alcohol levels in a single run. The method quantifies benzyl alcohol to 0.08% and ethyl acetate to 0.14% and detects benzyl alcohol to 0.03% and ethyl acetate to 0.04%.

Real Time In Vitro Release

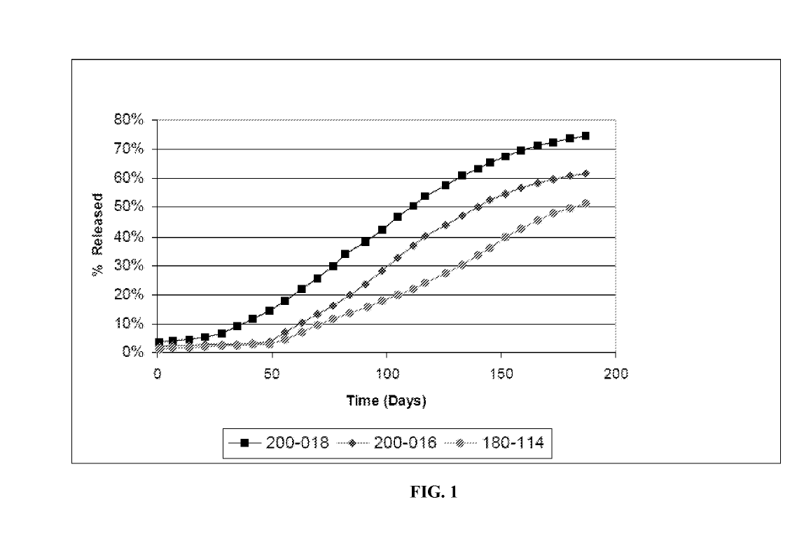

Extended release from microsphere formulations was determined in PBS (phosphate buffered saline pH 7.4, 0.05% Tween 20, and 0.05% sodium azide). Microparticle formulations (15 mg±1 mg) were weighed into a 1.5 mL polypropylene centrifuge tube (Fisher catalog#05 408 129) to which 1 mL of PBS was added. The tube was then placed in a 37° C. environmental chamber on a rotating mixer. The supernatant was sampled periodically by centrifuging at 2000 rpm, removing 1 mL, and replacing with fresh PBS. The removed sample was assayed for JPE1375 by RP-HPLC. The initial release was determined by the percentage of drug that was released in the first 24 hours of incubation. FIG. 1 shows in vitro real time release of JPE1375 PLGA sustained release formulations.

Microparticle Syringability

Syringeability of JPE1375 microsphere formulations was determined by weighing 20-40 mg of microspheres into a polypropylene microtube or glass vial. The ease of loading into and expelling formulation from a ½ cc syringe with 27 G ½″ needle was assessed upon suspending the microspheres in the vehicle (5% mannitol, 0.5% CMC and 0.05% Tween 20).

Differential Scanning Calorimetry (DSC)

DSC was performed to measure the polymer glass transition (T_{g}) of the microsphere formulations using a Differential Scanning calorimeter Q10 (TA instruments) and a heating rate of 10° C./min from 0° C. to 80° C.

### Example 4

### Injectable PLGA-Based Formulations for Sustained Delivery of Therapeutic Agents for Intraocular Applications

The C5aR peptidomimetic antagonist JPE1375 was encapsulated in PLGA microparticles using emulsion forming technology as described herein, and elsewhere, for example see WO 2005/003180. The microparticles were characterized for drug content and purity (HPLC), residual solvents (GC), and particle size (LLS). In vitro release kinetics and stability were measured in PBS (37° C.) by HPLC assay. In vitro bioactivity was accessed using rat basophil leukemia (RBL) cells transfected with human C5aR. The in vivo performance of select formulations was assessed after intravitreous injection in New Zealand rabbits by LC-MS.

Methods to determine in vitro release rate of the sustained release formulations, and in vivo release rates and concentrations are known in the art.

In Vivo Release of JPE1375 from JPE1375 PLGA Microparticles

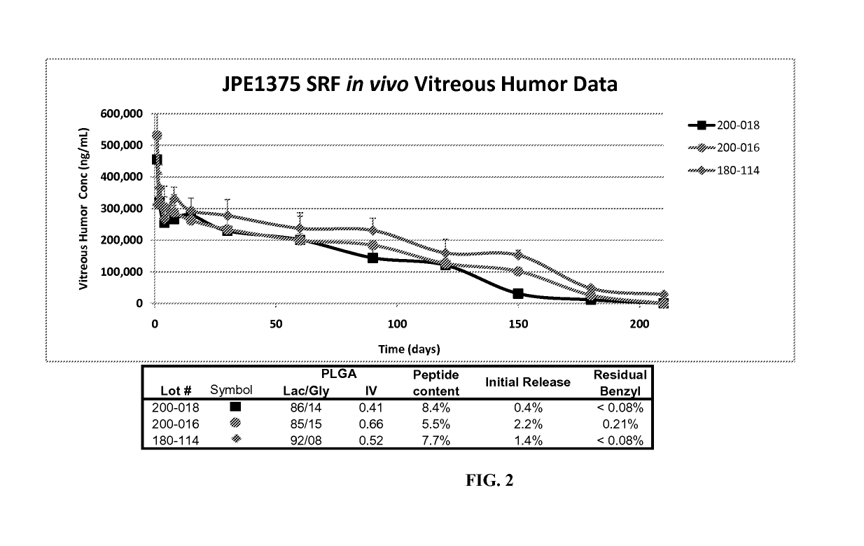

Test formulations (180-114, 200-016, 200-018) were prepared according to the methods described in the examples herein. The formulations were prepared using PLGA polymers of different inherent viscosity (surrogate for molecular weight) or lactide/glycolide ratios. These formulations were tested in vivo using New Zealand white rabbits. The formulations were suspended in a sterile filtered vehicle containing 5% mannitol, 0.5% CMC and 0.05% Tween 20 in water for injection. The suspended doses were administered to the rabbits by intravitreous injection. JPE1375 formulation doses were normalized to provide 500 micrograms of JPE1375 for each test formulation. The release of JPE1375 from the microspheres was assessed by measuring total JPE1375 content in the vitreous by a qualified LC-MS bioanalytical assay. A summary of this analysis for all three formulations is depicted in FIG. 2, which shows JPE1375 PLGA formulation release in vitreous humor.

JPE1375 concentrations, after administration of JPE1375 PLGA sustained release formulation, were also measured in whole retina tissue using a qualified LC-MS bioanalytical assay. A graphical summary of the data for the three test formulation is provided in FIG. 3, which shows in vivo retina tissue concentrations of JPE1375 after a single intravitreous administration of JPE1375 PLGA microparticle formulations.

The methods of making sustained release formulation provided defined microparticles with good drug contents (5-15%) and narrow particle size distribution compatible with injection through a narrow gauge needle, e.g. 27 gauge needle, compatible with intravitreous injection. Residual solvent levels were measured at low levels, ≦0.08-0.25% in the formulations. In vitro release kinetics showed a limited burst release (<5%) followed by PLGA dependent sustained release over a period of months, from 90 to more than 250 days. In vitro drug stability was supported by HPLC analysis of PBS release media and bioactivity in a C5aR bioassay. In vivo evaluation of formulations revealed systemic exposures were less than 0.1 ng/ml BLOQ at all time points while sustained retina levels of JPE1375 were maintained above the in vitro IC₅₀ for multiple weeks. The injectable PLGA-based formulations of the invention are a viable platform for delivery of JPE1375 to the posterior segment of the eye for months after intravitreous administration.

TABLE 5
Examples of formulations prepared by the methods as described in
Example 1 (lot#182-016) and Example 2 (lot#199-071).

| | | | | Peptide | 24 h/initial | Residual | Residual |
|---|---|---|---|---|---|---|---|
| | PLGA | PLGA | Yield | Content | release | Benzyl | ethyl |
| Lot# | lac/gly | IV | (% w/w) | (% w/w) | (%) | alcohol | acetate |
| 182-106 | 86/14 | 0.41 | 49% | 10.2% | 3.4% | <0.08%* | <0.04%* |
| 199-071 | 85/15 | 0.39 | 58% | | | <0.08%* | <0.04%* |
| 200-018 | 86/14 | 0.41 | | 8.4% | 0.4% | <0.08% | |
| 200-016 | 85/15 | 0.66 | | 5.5% | 2.2% | 0.21% | |
| 180-114 | 92/08 | 0.52 | | 7.7% | 1.4% | <0.08% | |

*below limit of quantitation

### Example 5

### Formulation Stability

Study Design

A six-month stability study was designed for JPE1375 microparticle lots 180-114, 200-016 and 200-018. The three lots differed by PLGA polymer type. Formulation 180-114 was prepared with a lactide to glycolide ratio of 92:08, an inherent viscosity (IV) of 0.52, and a carboxyl end group (90:10 5A Lakeshore #99-120-180). Formulation 200-016 was prepared with a lactide to glycolide ratio of 85:15, an IV of 0.66, and a carboxyl end group (85:15 6A Lakeshore #LP-255). Formulation 200-018 was prepared with a lactide to glycolide ratio of 86:14, an IV of 0.41, and a carboxyl end group (85:15 4A Lakeshore #97-12-201). Microparticle lots 200-016 and 200-018 were made with Fisher benzyl alcohol, lot 081699, and microparticle lot 180-114 was made with EMD benzyl alcohol, lot K34965681. The peptide content for formulations 180-114, 200-016 and 200-018 were 7.7%, 5.5% and 8.4% respectively.

Each lot was filled with a nitrogen blanket into 2 mL low volume glass vials (Mglas AG 37.5×16 mm, 37-02-2448-002), stoppered with butyl stoppers (West 4432/50 butyl, 10122185), and sealed with aluminum flip-off seals (West, 54130240). Each vial was filled with sufficient formulation to achieve a concentration of 500 μg JPE1375 per 50 μL injection when reconstituted with 150 μL injection vehicle, which varied from lot to lot based on the differences in measured peptide content. Vials were stored upright under two conditions, 5° C.±3° C., ambient relative humidity; and upright, 25° C.±2° C., 60%±5% relative humidity. The microparticles were analyzed for JPE1375 peptide content, purity, initial release, particle size and glass transition temperature. These analyses were performed at study start, 1, 3 and 6 months post study initiation.

Methods

Peptide Content/Purity

Formulation lots were assayed for JPE1375 content using a Waters 2695 HPLC system with a 2996 PDA detector. Approximately 5 mg of microparticle sample was weighed into a 2 mL plastic microcentrifuge tube. A 750 μL aliquot 5/95 DMSO/ACN was added to the tube. The tube was vortexed for 30 seconds and sonicated for 15 minutes to dissolve the sample. A 1250 μL aliquot of 0.1% TFA in water was added to the sample. The tube was vortexed for 30 seconds followed by centrifugation for 2 minutes at 14,000 RPM (20,800×g). The supernatant was analyzed by HPLC fitted with a Phenomenex C-18 Luna column (30×2 mm, 3 μm, 100 A) at 25° C. with a 50 μL injection volume, a 0.4 mL/min flow rate, and UV detection at 230 nm (spectra collected between 210-350 nm). The samples were analyzed using a gradient elution method as outlined in Table 10 below. A guard column containing a C18 cartridge was added to the method between the release (t=0) testing and the 1 month timepoint. This resulted in a shift in retention time of the standard by about 1 minute and minor changes in peak shape.

TABLE 10
HPLC method conditions

| Column: | Phenomenex C-18 Luna, 30 × 2 mm, | | |
|---|---|---|---|
| | 3 μm, 100 Å | | |
| Injection Volume: | 50ul | | |
| Flow Rate: | 0.4 mL/min | | |
| Mobile Phase: | A: 0.05% TFA in water | | |
| | B: 0.05% TFA in acetonitrile | | |
| | Time | % A | % B |
| Gradient: | 0.00 | 100 | 0 |
| | 1.00 | 100 | 0 |
| | 16.00 | 5 | 95 |
| | 21.00 | 5 | 95 |
| | 24.00 | 100 | 0 |
| | 30.00 | 100 | 0 |
| Column Temperature: | 25° C. | | |

In Vitro Release

Approximately 15 mg of microparticle sample was weighed in to a 1.5 mL plastic microcentrifuge tube. 1000 μL of pH 7.4 PBS (with 0.05% Tween-20 and 0.05% sodium azide) was added to the tube. The tube was vortexed for 3-5 seconds and placed on a rotisserie mixer rotating at 8 RPM in a 37° C. incubator. After 24 hours the tube was removed from the incubator and centrifuged for 2 minutes at 2,000 RPM. The supernatant was removed and filtered through a 0.2 μm PTFE filter and analyzed by HPLC (method above). After the supernatant was removed for the initial release measurement, 1000 μL, of pH 7.4 PBS (with 0.05% Tween-20 and 0.05% sodium azide) was added to the tube, and the tube was returned to the rotisserie in the incubator. Each week the tube was removed, centrifuged at 2,000 rpm and sampled as described above, and 1000 μL fresh PBS media was added back. Samples were visually inspected for particulate matter prior to HPLC analysis and filtered through a 0.2 μm PTFE filter as necessary for analysis.

Particle Size

Particle sizes were measured using a Coulter Particle Size Analyzer Optical Module LS 100Q with a Small Volume Module. Dry microparticles were added to the water reservoir until adequate light scattering obscuration was achieved. Coulter Non-Ionic Dispersant Type IC was used to uniformly suspend microparticles.

Glass Transition Temperature

Microparticle glass transition temperature was measured by differential scanning calorimetry (DSC) using a TA Instruments Differential Scanning calorimeter Q10. Approximately 5-10 mg sample was weighed into TA Instruments standard aluminum pans and placed in the instrument. The method equilibrated the sample at 0° C. and then ramped to 200° C. at 10° C./min.

Results

Peptide Content/Purity

The measured peptide content of all three JPE1375 lots were constant over 6 months and all were within 90-110% of the initial sample at 6 months (Table 11).

TABLE 11
Peptide Content

| | | 1-Month | | 3-Month | | 6-Month | |
|---|---|---|---|---|---|---|---|
| Lot | Initial | 5° C. | 25° C. | 5° C. | 25° C. | 5° C. | 25° C. |
| 180-114 | 7.7% | 7.2% | 7.2% | 7.2% | 7.2% | 7.3% | 7.3% |
| 200-016 | 5.5% | 5.3% | 5.2% | 5.4% | 5.4% | 5.5% | 5.4% |
| 200-018 | 8.4% | 8.1% | 7.9% | 8.0% | 8.1% | 8.4% | 8.4% |

The purity of 180-114 was higher initially than that of 200-016 and 200-018. This was attributed to the difference in benzyl alcohol lots used to manufacture 180-114 (EMD lot K34965681) versus 200-016 and 200-018 (Fisher lot 081699). HPLC analyses of the different sources of benzyl alcohol revealed a large difference in purity (Table 12). For the stability study, impurities that elute with relative retention time (RRT) the same as the benzyl alcohol peaks and that do not have UV spectrum comparable to JPE1375 have been ignored in the peptide purity analysis. Peaks that had a UV spectrum comparable to JPE1375 were included in the purity analysis.

TABLE 12
HPLC analysis of benzyl alcohol suppliers

| | Reten- | | | | | | |
|---|---|---|---|---|---|---|---|
| | tion | | | | Retention | | |
| | Time | RRT | Area % | | Time | RRT | Area % |
| EMD | 7.116 | 1.00 | 97.04% | Fisher | 7.135 | 1.00 | 79.89% |
| BnOH | 8.65 | 1.22 | 0.00% | BnOH | 8.697 | 1.22 | 3.87% |
| | 9.186 | 1.29 | 1.44% | | 9.207 | 1.29 | 13.49% |
| | 9.841 | 1.38 | 1.05% | | 9.818 | 1.38 | 2.13% |

The formulation peptide purity compared to the initial time point of all three JPE1375 formulations remained stable over 6 months (Table 13). The purity for lot 180-114 was between 95.0-105.0% of the peptide standard at all time points. The purities for both 200-016 and 200-018 were less than 95.0% of the peptide standard; however the purity remained constant (95.0%<purity<105%) relative to the initial time point over 6 months at both the 5° C. or 25° C. storage conditions.

TABLE 13
Formulation peptide purity

| | | 1-Month | | 3-Month | | 6-Month | |
|---|---|---|---|---|---|---|---|
| Lot | Initial | 5° C. | 25° C. | 5° C. | 25° C. | 5° C. | 25° C. |
| 180- | 97.9% | 99.4% | 96.1% | 97.7% | 97.5% | 97.3% | 96.8% |
| 114 | | | | | | | |
| 200- | 83.2% | 84.5% | 83.1% | 85.9% | 85.0% | 85.6% | 84.9% |
| 016 | | | | | | | |
| 200- | 89.0% | 88.2% | 87.4% | 89.8% | 89.7% | 89.2% | 90.2% |
| 018 | | | | | | | |

In Vitro Release

Changes were measured in the initial release over 6 months, up to a 1% increase in the initial amount of released peptide (Table 14). Due to the low initial release (burst release) these changes are large relative changes but are small relative to total peptide content. In addition the variation measured is similar to the experimental error for the method used for this analysis. Therefore it is concluded that there is no significant change in initial in vitro release over 6 months.

TABLE 14
In vitro initial peptide release.

| | | 1-Month | | 3-Month | | 6-Month | |
|---|---|---|---|---|---|---|---|
| Lot | Initial | 5° C. | 25° C. | 5° C. | 25° C. | 5° C. | 25° C. |
| 180- | 1.52% | 1.95% | 2.33% | 2.04% | 2.25% | 2.23% | 2.56% |
| 114 | | | | | | | |
| 200- | 2.52% | 2.80% | 2.91% | 3.23% | 2.86% | 3.15% | 2.98% |
| 016 | | | | | | | |
| 200- | 4.00% | 5.90% | 4.40% | 5.51% | 5.14% | 4.58% | 5.07% |
| 018 | | | | | | | |

The in vitro real time extended release of the formulations was measured after formulation fabrication. The in vitro real time release after 6 months storage at each stability condition will be measured.

Particle Size

The mean particle size of the formulations increased over 6 months, resulting in a larger percentage of microparticles greater than 63 μm (Table 15). This increase was greater at the 25° C. condition relative to the 5° C. stability condition. During the fabrication process the formulation microparticles were collected between sieves of 25 and 63 μm to control the particle size distribution. Formulations containing particle above 63 μm in diameter are likely caused by aggregation of the particles under the stability storage conditions. Sonication of the formulation stability samples resulted in return of the formulations to their initial mean particle size and distribution. This result demonstrates the increased size is not due to irreversible degradation of the formulations.

TABLE 15
Formulation Mean and Particle Size Distribution

| | | | | 6-Month | |
|---|---|---|---|---|---|
| Lot | | | Initial | 5° C. | 25° C. |
| 180-114 | Mean Particle Size | | 34.8 μm | 37.0 μm | 46.4 μm |
| | | | | (34.2 μm) | (35.2 μm) |
| | Volume % | <25 μm | 19% | 17% | 13% |
| | | | | (20%) | (19%) |
| | | 25-63 μm | 80% | 77% | 67% |
| | | | | (79%) | (78%) |
| | | >63 μm | 1% | 6% | 20% |
| | | | | (1%) | (3%) |
| 200-016 | Mean Particle Size | | 41.2 μm | 43.8 μm | 56.0 μm |
| | | | | (40.4 μm) | (41.5 μm) |
| | Volume % | <25 μm | 2% | 2% | 4% |
| | | | | (3%) | (3%) |
| | | 25-63 μm | 96% | 91% | 68% |
| | | | | (96%) | (95%) |
| | | >63 μm | 2% | 7% | 28% |
| | | | | (1%) | (2%) |
| 200-018 | Mean Particle Size | | 38.5 μm | 42.6 μm | 50.5 μm |
| | | | | (38.8 μm) | (39.1 μm) |
| | Volume % | <25 μm | 2% | 0% | 0% |
| | | | | (1%) | (3%) |
| | | 25-63 μm | 98% | 92% | 78% |
| | | | | (99%) | (95%) |
| | | >63 μm | 0% | 8% | 22% |
| | | | | (0%) | (2%) |

Note:
Values in parenthesis were obtained by measuring formulation particle size after sonication of the original sample.

Glass Transition Temperature

The glass transition temperature of formulations stored at the 5° C. stability condition did not change over the course of the 6 month stability time frame (Table 16). The glass transition temperature for formulations stored at the 25° C. stability condition increased by several degrees centigrade for each formulation.

TABLE 16

| | | 1-Month | | 3-Month | | 6-Month | |
|---|---|---|---|---|---|---|---|
| Lot | Initial | 5° C. | 25° C. | 5° C. | 25° C. | 5° C. | 25° C. |
| 180-114 | 55.1° C. | 55.2° C. | 57.3° C. | 55.7° C. | 58.7° C. | 55.7° C. | 58.9° C. |
| 200-016 | 52.4° C. | 51.4° C. | 55.5° C. | 53.0° C. | 57.0° C. | 53.2° C. | 58.6° C. |
| 200-018 | 53.9° C. | 53.8° C. | 56.7° C. | 54.6° C. | 58.4° C. | 54.3° C. | 58.7° C. |

Hypothetical Examples of In Vivo Efficacy and Treatment

Immune Complex Peritonitis

C5a is a potent chemokine, inducing polymorphic nucleated granulocyte (neutrophils) and monocyte/macrophage chemotaxis. In the Reverse Passive Arthus Reaction model in mice, an immune complex peritonitis is induced by the intravenous administration of ovalbumin followed by intraperitoneal administration of an anti-ovalbumin antiserum, which leads to the formation of antigen-antibody complexes and subsequent activation of the complement system, including generation of C5a [Heller et al., 1999]. (Heller et al (1999) J Immunol 163; 985-994). JPE1375 demonstrates high in vivo potency for the inhibition of C5a receptor mediated chemotaxis in this model following an intraperitoneal injection of 1 mg/kg as a liquid formulation. Sustained release microparticle formulations containing JPE1375 dosed by intraperitoneal injection are expected to demonstrate similar activity in this model.

Kidney Transplantation

Complement-related renal ischemia/reperfusion injury has been studied in a variety of animal models and various inhibitors of complement activation, including other C5a antagonists, have been shown to prevent local ischemia/reperfusion tissue injury in the kidney (Arumugam et al., 2003; De Vries et al., 2003a, b). (Arumugam, T. V., Shiels I. A., Strachan, A. J., et al (2003) A small molecule C5a receptor antagonist protects kidneys from ischemia/reperfusion injury in rats. Kidney Int 63; 134-142, De Vries B, Matthijsen R A, Wolfs T G et al (2003a) Inhibition of complement factor C5 protects against renal ischemia-reperfusion injury: inhibition of late apoptosis and inflammation Transplantation 75; 375-382, De Vries B, Kohl J, Leclercq W K et al (2003b) Complement factor C5a mediates renal ischemia-reperfusion injury independent of neutrophils. J Immunol 170; 3883-3889.) JPE1375 liquid formulation is active in an allogenic transplantation model in mice. In this model rejection of the transplant is based on an ischemia reperfusion injury with measures of animal/graft survival and renal function. Blockade of C5aR with JPE1375 liquid formulation after transplantation prolonged survival of the animals from days to greater than 12 weeks compared to controls. C5aR blockade with JPE1375 microparticle sustained release formulation is expected to provide similar benefit after a single dose.

Rheumatoid Arthritis

Activation of the complement system is known to play a role in autoimmune and inflammatory diseases including rheumatoid arthritis (RA) (Okroj, M., Heinegard, D., Holmdahl, R. et al (2007) Rheumatoid arthritis and the complement system. Annals of Medicine 39, 517-530). A number of animal models have been developed to model RA and evaluate active agents during nonclinical development (Joe, B., Wilder, R L. (1995) Animal models of rheumatoid arthritis. Mol. Med. Today, 5, 376-369. Bendele, A M (2001) Animal models of rheumatoid arthritis. J. Musculoskel Neuoron Interact 1, 377-385). The mouse and rat collagen type II model is an antigen mediated complement system activation model the mimics the histopathology of human disease. Sustained release microparticle formulations that release the C5aR antagonist JPE175 is expected to demonstrate activity and have therapeutic effect in an animal model of RA.

### APPENDIX A

TABLE 1
Summary of several sustained release formulations

| | | | | | Drug Release |
|---|---|---|---|---|---|
| Product Name | Distributor | Active ingredient | Activity | Formulation | (months) |
| Lupron ® Depot | TAP | Luprorelin (peptide) | LHRH agonist | microparticles | 1, 3, 4 |
| Luprogel ® | MediGene AG | Luprorelin (peptide) | LHRH agonist | solid bolus | 1 |
| Eligard ® | Sanofi-Synthelabo | Luprorelin (peptide) | LHRH agonist | solid bolus | 1, 3 |
| Decapeptyl ® | Ferring | Triptorelin | LHRH agonist | microparticles | 1 |
| | | (peptide) | | | |
| Decapeptyl LP | Ipsen_beaufour | Triptorelin | LHRH agonist | microparticles | 1, 3 |
| | | (peptide) | | | |
| Trelstar ® Depot | Pfizer | Triptorelin | LHRH agonist | microparticles | 1, 3 |
| | | (peptide) | | | |
| Suprecur ® MP | Aventis | Buserelin (peptide) | LHRH agonist | microparticles | 1 |
| Profact ® Depot (implant) | Aventis | Buserelin (peptide) | LHRH agonist | implant (rod) | 2, 3 |
| Zoladex ® (implant) | AstraZeneca | Goserelin (peptide) | LHRH agonist | implant (rod) | 1, 3 |
| Sandostatin LAR ® Depot | Novartis | Octreotide | Somatostatin | microparticles | 1 |
| | | (peptide) | antagonist | | |
| Somatuline ® LA | Ispen-Beaufour | Lanreotide | Somatostatin | microparticles | 1 |
| | | (peptide) | antagonist | | |
| Nutropin Depot ® | Genentech | Growth hormone | protein | microparticles | 0.5, 1 |
| | | (protein) | replacement | | |
| Arestin ® | OraPharma | Minocycline (small | Tetracycline | microparticles | 0.5 |
| | | molecule) | antibiotic | | |
| Atridox ® | CollaGenex | Doxycycline (small | Tetracycline | solid bolus | 0.25 |
| | | molecule) | antibiotic | | |
| Risperdal ® Consta ™ | J&J | Risperidone (small | Dopamine | microparticles | 0.5 |
| | | molecule) | antagonist | | |
| Vivitrol | Cephalon/Alkermes | Naltrexone (small | Opioid | microparticles | 1 |
| | | molecule) | antagonist | | |
| SMARTShot B12 | Stockguard Labs | Vitamine B12 | diet supplement | microparticles | 4, 8 |

TABLE 2
Experimental conditions and results for microparticle formulations made
using dichloromethane and benzyl alcohol

| | | | | Mean | | Target | | Residual |
|---|---|---|---|---|---|---|---|---|
| | Oil Phase | Oil Phase | Quench | Particle | | Drug | Drug | Benzyl |
| | DCM:BA | Conc. | Conditions. | Size | Yield | Content | Content | Alcohol |
| Lot # | Ratio | (w/v) | (0.3% PVA) | (μm) | (w/w) | (w/w) | (w/w) | (w/w) |
| 163 006 1 | 1:0.9 | 5.3% | 50 mL/min | 16 | 75% | 5% | 3.3% | *ND |
| 163 006 2 | 9:1 | 10.0% | 50 mL/min | 19 | 48% | 5% | 0.5% | *ND |
| 163 013 1 | 9:1 | 20.0% | 50 mL/min | 36 | 39% | 5% | 1.0% | *ND |
| 163 017 1 | 2.5:1 | 2.9% | 50 mL/min | 19 | 94% | 5% | 0.0% | *ND |
| 163 017 2 | 1:1 | 20.0% | 50 mL/min | 74 | 51% | 5% | 2.4% | *ND |
| 163 027 1 | 1:1 | 5.0% | 50 mL/min | 24 | 102% | 5% | 3.3% | 2.5% |
| 163 027 2 | 1:1 | 2.5% | 50 mL/min | 87 | 84% | 5% | 1.7% | *ND |
| 163 045 2 | 1:1 | 5.0% | 50 mL/min | 59 | 69% | 10% | 5.1% | 2.9% |

*ND = not determined.

TABLE 3
Experimental conditions and results for microparticle formulations made using ethyl acetate and benzyl alcohol.

| | | | | | | | Target | | | Residual | Residual | |
|---|---|---|---|---|---|---|---|---|---|---|---|---|
| | | | Additional | | | | Drug | Peptide | Drug | Benzyl | Ethyl | Initial |
| | PLGA | | Processing | | Yield | Tg | Content | Content | Content | Alcohol | Acetate | Release |
| Lot # | Lac/Gly | IV | Wash | Heating | (w/w) | (° C.) | (w/w) | (w/w) | (w/w) | (w/w) | (w/w) | (w/w) |
| 163 099 2 | 92/08 | 0.52 | 10 DV | 45 C. | 48% | ND | 10% | ND | 5.5% | 2.4% | *ND | 0.2% |
| 163 105 1 | 85/15 | 0.95 | 6 DV | 40 C. | NA | ND | 10% | ND | 4.5% | 3.7% | *ND | 0.3% |
| 163 105 2 | 85/15 | 0.95 | 8 DV | 40 C. | 40% | ND | 10% | ND | 4.0% | 2.1% | *ND | 0.2% |
| 163 111 1 | 100/0 | 0.24 | 5 DV | 40 C. | 16% | ND | 10% | ND | 2.3% | 0.2% | *ND | 1.1% |
| 163 117 1 | 76/24 | 0.46 | 10 DV | 40 C. | 53% | ND | 10% | ND | 6.0% | 2.6% | *ND | 0.3% |
| 163 123 1 | 86/14 | 0.41 | 6 DV | 40 C. | NA | ND | 10% | ND | 6.5% | 3.4% | 0.1% | 1.2% |
| 163 123 2 | 86/14 | 0.41 | 8 DV | 50 C. | 61% | ND | 10% | ND | 6.3% | 1.4% | <0.04%** | 0.5% |
| 163 128 1 | 86/14 | 0.41 | 6 DV | 40 C. | NA | ND | 10% | ND | 7.0% | 3.7% | <0.04%** | 0.3% |
| 163 128 2 | 86/14 | 0.41 | 6 DV | 40 C./2 hr | 73% | ND | 10% | ND | 7.1% | 1.5% | <0.04%** | 2.0% |
| 163 134 1 | 86/14 | 0.41 | 6 DV | 40 C. | NA | ND | 15% | ND | 10.7% | 3.9% | <0.04%** | 3.2% |
| 163 134 2 | 86/14 | 0.41 | 6 DV | 40 C./3 hr | 63% | ND | 15% | ND | 10.2% | 0.5% | <0.04%** | 2.6% |
| 163 141 1 | 85/15 | 0.66 | 6 DV | 40 C./3 hr | 48% | ND | 15% | ND | 9.5% | 0.6% | <0.04%** | 1.7% |
| 163 146 1 | 86/14 | 0.41 | 6 DV | 40 C./3 hr | 59% | ND | 15% | ND | 8.9% | 1.1% | 0.1% | 0.2% |
| 163 150 1 | 86/14 | 0.41 | 5 DV | 40 C./3 hr | 55% | ND | 15% | ND | 9.9% | 1.1% | <0.04%** | 1.1% |
| 163 154 1 | 85/15 | 0.66 | 5 DV | 40 C./3.5 hr | 44% | ND | 15% | ND | 9.0% | 1.5% | <0.04%** | 0.1% |
| 163 158 1 | 86/14 | 0.41 | 10 DV | 40 C./4 hr | NA | 49.9 | 15% | ND | 10.4% | 1.4% | <0.04%** | 2.3% |
| 163 158 2 | 86/14 | 0.41 | 13 DV | 40 C./27 hr | 50% | 54.5 | 15% | ND | 8.6% | 0.13% | <0.04%** | 2.5% |
| 163-164 1 | 90/10 | 0.27 | 10 DV | 40 C./3 hr | NA | 44.3 | 15% | ND | 6.8% | 1.30% | <0.04%** | 3.8% |
| 163-164 2 | 90/10 | 0.27 | 16 DV | 40 C./24 hr | 57% | 47.6 | 15% | ND | 6.1% | 0.10% | <0.04%** | 7.8% |
| 163-172 1 | 92/08 | 0.52 | 10 DV | 40 C./24 hr | NA | 56.9 | 15% | ND | 8.8% | 0.46% | <0.04%** | 0.6% |
| 163-172 2 | 92/08 | 0.52 | 20 DV | 45 C./5 hr | 45% | 56.5 | 15% | ND | 8.6% | <0.08%* | <0.04%** | 2.1% |
| 163-182 1 | 85/15 | 0.66 | 16 DV | 40 C./23 hr | 40% | 55.3 | 15% | ND | 8.3% | 0.58% | <0.04%** | 0.5% |
| 163-188 1 | 86/14 | 0.41 | 10 DV | 40 C./4 hr | NA | 55.5 | 15% | 8.0% | 8.9% | <0.08%* | <0.04%** | 0.7% |
| 163-188 2 | 86/14 | 0.41 | 10 DV | 48 C./1 hr | 49% | 56.0 | 15% | 7.8% | 8.7% | <0.03%** | <0.04%** | 0.9% |
| 180-001 1 | 86/14 | 0.41 | 0 DV | *NA | NA | ND | 20% | 9.8% | 10.9% | ND | ND | ND |
| 180-001 2 | 86/14 | 0.41 | 8 DV | 39 C./3 hr | 54% | 55.5 | 20% | 10.3% | 11.4% | <0.03%** | <0.04%** | 5.4% |
| 180-008 1 | 86/14 | 0.41 | 8 DV¹ | 39 C./1.5 hr | 57% | 54.3 | 20% | 10.3% | 11.4% | <0.08%* | <0.04%** | 1.6% |
| 180-014 1² | 86/14 | 0.41 | 8 DV¹ | 39 C./1.5 hr | 54% | 55.4 | 20% | 10.4% | 11.6% | <0.03%** | <0.04%** | 3.4% |
| 180-019 1² | 85/15 | 0.66 | 8 DV¹ | 39 C./1.5 hr | 55% | 55.2 | 20% | 7.7% | 8.6% | <0.08%* | <0.04%** | 1.8% |
| 180-024-1² | 92/08 | 0.52 | 8 DV¹ | 39 C./1.5 hr | NA | 56.8 | 20% | 8.8% | 9.8% | <0.03%** | <0.04%** | 3.0% |
| 180-024-2² | 92/08 | 0.52 | 8 DV¹ | 39 C./1.5 hr | 40% | 56.8 | 20% | 8.8% | 9.7% | <0.08%* | <0.04%** | 3.0% |
| 180-030-1² | 85/15 | 0.66 | 12 DV¹ | 39 C./1.5 hr | 54% | 54.7 | 20% | 7.5% | 8.3% | 0.20% | <0.04%** | 4.7% |
| 180-038-1² | 85/15 | 0.66 | 10 DV¹ | 39 C./1.5 hr | 29% | 55.2 | 20% | 6.6% | 7.40% | <0.08%* | <0.04%** | 9.3% |

TBD = to be determined,
¹= No PVA in primary extraction water,
²= No ethyl acetate in water phase,
ND = value not determined,
NA = value not applicable,
*= Below limit of quantitation,
**= Below limit of detection
TBD = to be determined,
¹= no PVA in primary extraction,
²= no ethyl acetate in aqueous phase,
ND = not determined,
NA = not applicable,
*= below limit of quantification,
**= below limit of detection

TABLE 6
JPE1375 plasma concentrations from formulation 180-114 measured by
a validated LC-MS assay

| | | | Calculated | Calibration |
|---|---|---|---|---|
| | | Timepoint | Concentration | Curve range |
| Matrix | Formulation | (Day) | (ng/mL) | (ng/mL) |
| Plasma | 180-114 | −1 | BLQ | 0.46-920 |
| Plasma | 180-114 | −1 | BLQ | 0.46-920 |
| Plasma | 180-114 | 2 | BLQ | 0.46-920 |
| Plasma | 180-114 | 2 | BLQ | 0.46-920 |
| Plasma | 180-114 | 3 | BLQ | 0.46-920 |
| Plasma | 180-114 | 3 | BLQ | 0.46-920 |
| Plasma | 180-114 | 5 | BLQ | 0.46-920 |
| Plasma | 180-114 | 5 | BLQ | 0.46-920 |
| Plasma | 180-114 | 9 | BLQ | 0.46-920 |
| Plasma | 180-114 | 9 | BLQ | 0.46-920 |
| Plasma | 180-114 | 16 | BLQ | 0.46-920 |
| Plasma | 180-114 | 16 | BLQ | 0.46-920 |
| Plasma | 180-114 | 31 | BLQ | 0.46-920 |
| Plasma | 180-114 | 31 | BLQ | 0.46-920 |
| Plasma | 180-114 | 60 +/− 1 | BLQ | 0.46-920 |
| Plasma | 180-114 | 60 +/− 1 | BLQ | 0.46-920 |
| Plasma | 180-114 | 90 +/− 1 | BLQ | 0.46-920 |
| Plasma | 180-114 | 90 +/− 1 | BLQ | 0.46-920 |
| Plasma | 180-114 | 121 +/− 1 | BLQ | 0.46-920 |
| Plasma | 180-114 | 121 +/− 1 | BLQ | 0.46-920 |
| Plasma | 180-114 | 150 +/− 2 | BLQ | 0.46-920 |
| Plasma | 180-114 | 150 +/− 2 | BLQ | 0.46-920 |
| Plasma | 180-114 | 180 +/− 2 | BLQ | 0.46-920 |
| Plasma | 180-114 | 180 +/− 2 | BLQ | 0.46-920 |

TABLE 7
JPE1375 plasma concentrations from formulation 200-016 measured by
a validated LC-MS assay

| | | | | Calibration |
|---|---|---|---|---|
| | | | Calculated | Curve |
| | | Timepoint | Concentration | range |
| Matrix | Formulation | (Day) | (ng/mL) | (ng/mL) |
| Plasma | 200-016 | −1 | BLQ | 0.46-920 |
| Plasma | 200-016 | −1 | BLQ | 0.46-920 |
| Plasma | 200-016 | 2 | BLQ | 0.46-920 |
| Plasma | 200-016 | 2 | BLQ | 0.46-920 |
| Plasma | 200-016 | 3 | BLQ | 0.46-920 |
| Plasma | 200-016 | 3 | BLQ | 0.46-920 |
| Plasma | 200-016 | 5 | BLQ | 0.46-920 |
| Plasma | 200-016 | 5 | BLQ | 0.46-920 |
| Plasma | 200-016 | 9 | BLQ | 0.46-920 |
| Plasma | 200-016 | 9 | BLQ | 0.46-920 |
| Plasma | 200-016 | 16 | BLQ | 0.46-920 |
| Plasma | 200-016 | 16 | BLQ | 0.46-920 |
| Plasma | 200-016 | 31 | BLQ | 0.46-920 |
| Plasma | 200-016 | 31 | BLQ | 0.46-920 |
| Plasma | 200-016 | 60 +/− 1 | BLQ | 0.46-920 |
| Plasma | 200-016 | 60 +/− 1 | BLQ | 0.46-920 |
| Plasma | 200-016 | 90 +/− 1 | BLQ | 0.46-920 |
| Plasma | 200-016 | 90 +/− 1 | BLQ | 0.46-920 |
| Plasma | 200-016 | 121 +/− 1 | BLQ | 0.46-920 |
| Plasma | 200-016 | 121 +/− 1 | BLQ | 0.46-920 |
| Plasma | 200-016 | 150 +/− 2 | BLQ | 0.46-920 |
| Plasma | 200-016 | 150 +/− 2 | BLQ | 0.46-920 |
| Plasma | 200-016 | 180 +/− 2 | BLQ | 0.46-920 |
| Plasma | 200-016 | 180 +/− 2 | BLQ | 0.46-920 |

TABLE 8
JPE1375 plasma concentrations from formulation 200-018 measured by
a validated LC-MS assay

| | | | | Calibration |
|---|---|---|---|---|
| | | | Calculated | Curve |
| | | Timepoint | Concentration | range |
| Matrix | Formulation | (Day) | (ng/mL) | (ng/mL) |
| Plasma | 200-018 | −1 | BLQ | 0.46-920 |
| Plasma | 200-018 | −1 | BLQ | 0.46-920 |
| Plasma | 200-018 | 2 | BLQ | 0.46-920 |
| Plasma | 200-018 | 2 | BLQ | 0.46-920 |
| Plasma | 200-018 | 3 | BLQ | 0.46-920 |
| Plasma | 200-018 | 3 | BLQ | 0.46-920 |
| Plasma | 200-018 | 5 | BLQ | 0.46-920 |
| Plasma | 200-018 | 5 | BLQ | 0.46-920 |
| Plasma | 200-018 | 9 | BLQ | 0.46-920 |
| Plasma | 200-018 | 9 | BLQ | 0.46-920 |
| Plasma | 200-018 | 16 | BLQ | 0.46-920 |
| Plasma | 200-018 | 16 | BLQ | 0.46-920 |
| Plasma | 200-018 | 31 | BLQ | 0.46-920 |
| Plasma | 200-018 | 31 | BLQ | 0.46-920 |
| Plasma | 200-018 | 60 +/− 1 | BLQ | 0.46-920 |
| Plasma | 200-018 | 60 +/− 1 | BLQ | 0.46-920 |
| Plasma | 200-018 | 90 +/− 1 | BLQ | 0.46-920 |
| Plasma | 200-018 | 90 +/− 1 | BLQ | 0.46-920 |
| Plasma | 200-018 | 121 +/− 1 | BLQ | 0.46-920 |
| Plasma | 200-018 | 121 +/− 1 | BLQ | 0.46-920 |
| Plasma | 200-018 | 150 +/− 2 | BLQ | 0.46-920 |
| Plasma | 200-018 | 150 +/− 2 | BLQ | 0.46-920 |
| Plasma | 200-018 | 180 +/− 2 | BLQ | 0.46-920 |
| Plasma | 200-018 | 180 +/− 2 | BLQ | 0.46-920 |

TABLE 9
JPE1375 retina tissue concentrations measured by a qualified LC-MS
assay

| | | | Calculated | Calibration |
|---|---|---|---|---|
| | | Timepoint | Concentration | Curve range |
| Matrix | Formulation | (Day) | (ng/mL) | (ng/mL) |
| Retina | 180-114 | 1 | 36.4 | 0.0460-92.0 |
| Retina | 180-114 | 1 | 3.18 | 0.0460-92.0 |
| Retina | 180-114 | 1 | 121 | 0.0460-92.0 |
| Retina | 180-114 | 1 | 13.0 | 0.0460-92.0 |
| Retina | 180-114 | 2 | 1550 | 0.0460-92.0 |
| Retina | 180-114 | 2 | 290 | 0.0460-92.0 |
| Retina | 180-114 | 2 | 86.4 | 0.0460-92.0 |
| Retina | 180-114 | 2 | 632 | 0.0460-92.0 |
| Retina | 180-114 | 4 | 251 | 0.0460-92.0 |
| Retina | 180-114 | 4 | 40.6 | 0.0460-92.0 |
| Retina | 180-114 | 4 | 27.2 | 0.0460-92.0 |
| Retina | 180-114 | 4 | 10.8 | 0.0460-92.0 |
| Retina | 180-114 | 8 | 20.2 | 0.0460-92.0 |
| Retina | 180-114 | 8 | 155 | 0.0460-92.0 |
| Retina | 180-114 | 8 | 143 | 0.0460-92.0 |
| Retina | 180-114 | 8 | 1780 | 0.0460-92.0 |
| Retina | 180-114 | 15 | 4390 | 0.0460-92.0 |
| Retina | 180-114 | 15 | 705 | 0.0460-92.0 |
| Retina | 180-114 | 15 | 65.7 | 0.0460-92.0 |
| Retina | 180-114 | 15 | 80.0 | 0.0460-92.0 |
| Retina | 180-114 | 30 | 332 | 0.0460-92.0 |
| Retina | 180-114 | 30 | 338 | 0.0460-92.0 |
| Retina | 180-114 | 30 | 120 | 0.0460-92.0 |
| Retina | 180-114 | 30 | 498 | 0.0460-92.0 |
| Retina | 180-114 | 60 +/− 1 | 3809 | 0.0460-92.0 |
| Retina | 180-114 | 60 +/− 1 | 1205 | 0.0460-92.0 |
| Retina | 180-114 | 60 +/− 1 | 554 | 0.0460-92.0 |
| Retina | 180-114 | 60 +/− 1 | 5207 | 0.0460-92.0 |
| Retina | 180-114 | 90 +/− 1 | 912 | 0.460-2300 |
| Retina | 180-114 | 90 +/− 1 | 880 | 0.460-2300 |
| Retina | 180-114 | 90 +/− 1 | 2130 | 0.460-2300 |
| Retina | 180-114 | 90 +/− 1 | 2290 | 0.460-2300 |
| Retina | 180-114 | 121 +/− 1 | 12400 | 0.460-2300 |
| Retina | 180-114 | 121 +/− 1 | 459 | 0.460-2300 |
| Retina | 180-114 | 121 +/− 1 | 1320 | 0.460-2300 |
| Retina | 180-114 | 121 +/− 1 | 1620 | 0.460-2300 |
| Retina | 180-114 | 150 +/− 2 | 1140 | 0.460-2300 |
| Retina | 180-114 | 150 +/− 2 | 408 | 0.460-2300 |
| Retina | 180-114 | 180 +/− 2 | 85.0 | 0.460-2300 |
| Retina | 180-114 | 180 +/− 2 | 207 | 0.460-2300 |
| Retina | 180-114 | 180 +/− 2 | 840 | 0.460-2300 |
| Retina | 180-114 | 180 +/− 2 | 76.2 | 0.460-2300 |
| Retina | 200-016 | 1 | 5.60 | 0.0460-92.0 |
| Retina | 200-016 | 1 | 68.5 | 0.0460-92.0 |
| Retina | 200-016 | 1 | 2.82 | 0.0460-92.0 |
| Retina | 200-016 | 1 | 235 | 0.0460-92.0 |
| Retina | 200-016 | 2 | 27.0 | 0.0460-92.0 |
| Retina | 200-016 | 2 | 131 | 0.0460-92.0 |
| Retina | 200-016 | 2 | 61.2 | 0.0460-92.0 |
| Retina | 200-016 | 2 | 11.5 | 0.0460-92.0 |
| Retina | 200-016 | 4 | 302 | 0.0460-92.0 |
| Retina | 200-016 | 4 | 2730 | 0.0460-92.0 |
| Retina | 200-016 | 4 | 1040 | 0.0460-92.0 |
| Retina | 200-016 | 4 | 3740 | 0.0460-92.0 |
| Retina | 200-016 | 8 | 1.86 | 0.0460-92.0 |
| Retina | 200-016 | 8 | 74.4 | 0.0460-92.0 |
| Retina | 200-016 | 8 | 66.1 | 0.0460-92.0 |
| Retina | 200-016 | 8 | 11.4 | 0.0460-92.0 |
| Retina | 200-016 | 15 | 698 | 0.0460-92.0 |
| Retina | 200-016 | 15 | 172 | 0.0460-92.0 |
| Retina | 200-016 | 15 | 5.66 | 0.0460-92.0 |
| Retina | 200-016 | 15 | 12.3 | 0.0460-92.0 |
| Retina | 200-016 | 30 | 284 | 0.0460-92.0 |
| Retina | 200-016 | 30 | 1910 | 0.0460-92.0 |
| Retina | 200-016 | 30 | 128 | 0.0460-92.0 |
| Retina | 200-016 | 30 | 89.4 | 0.0460-92.0 |
| Retina | 200-016 | 60 +/− 1 | 1812 | 0.0460-92.0 |
| Retina | 200-016 | 60 +/− 1 | 3238 | 0.0460-92.0 |
| Retina | 200-016 | 60 +/− 1 | 3386 | 0.0460-92.0 |
| Retina | 200-016 | 60 +/− 1 | 994 | 0.0460-92.0 |
| Retina | 200-016 | 90 +/− 1 | 8250 | 0.460-2300 |
| Retina | 200-016 | 90 +/− 1 | 4810 | 0.460-2300 |
| Retina | 200-016 | 90 +/− 1 | 130 | 0.460-2300 |
| Retina | 200-016 | 90 +/− 1 | 463 | 0.460-2300 |
| Retina | 200-016 | 121 +/− 1 | 130 | 0.460-2300 |
| Retina | 200-016 | 121 +/− 1 | 316 | 0.460-2300 |
| Retina | 200-016 | 121 +/− 1 | 674 | 0.460-2300 |
| Retina | 200-016 | 121 +/− 1 | 24.8 | 0.460-2300 |
| Retina | 200-016 | 150 +/− 2 | 9.94 | 0.460-2300 |
| Retina | 200-016 | 150 +/− 2 | 0.0 | 0.460-2300 |
| Retina | 200-016 | 180 +/− 2 | 8.31 | 0.460-2300 |
| Retina | 200-016 | 180 +/− 2 | 1950 | 0.460-2300 |
| Retina | 200-016 | 180 +/− 2 | 0.0 | 0.460-2300 |
| Retina | 200-016 | 180 +/− 2 | 0.0 | 0.460-2300 |
| Retina | 200-018 | 1 | 5.12 | 0.0460-92.0 |
| Retina | 200-018 | 1 | 7.69 | 0.0460-92.0 |
| Retina | 200-018 | 1 | 3.99 | 0.0460-92.0 |
| Retina | 200-018 | 1 | 6.05 | 0.0460-92.0 |
| Retina | 200-018 | 2 | 11.1 | 0.0460-92.0 |
| Retina | 200-018 | 2 | 55.0 | 0.0460-92.0 |
| Retina | 200-018 | 2 | 76.7 | 0.0460-92.0 |
| Retina | 200-018 | 2 | 6.28 | 0.0460-92.0 |
| Retina | 200-018 | 4 | 0.594 | 0.0460-92.0 |
| Retina | 200-018 | 4 | 49.7 | 0.0460-92.0 |
| Retina | 200-018 | 4 | 11.6 | 0.0460-92.0 |
| Retina | 200-018 | 4 | 28.2 | 0.0460-92.0 |
| Retina | 200-018 | 8 | 26.9 | 0.0460-92.0 |
| Retina | 200-018 | 8 | 20.6 | 0.0460-92.0 |
| Retina | 200-018 | 8 | 27508.0 | 0.0460-92.0 |
| Retina | 200-018 | 8 | 59.2 | 0.0460-92.0 |
| Retina | 200-018 | 15 | 3.26 | 0.0460-92.0 |
| Retina | 200-018 | 15 | 8.73 | 0.0460-92.0 |
| Retina | 200-018 | 15 | 4830 | 0.0460-92.0 |
| Retina | 200-018 | 15 | 39.3 | 0.0460-92.0 |
| Retina | 200-018 | 30 | 302 | 0.0460-92.0 |
| Retina | 200-018 | 30 | 609 | 0.0460-92.0 |
| Retina | 200-018 | 30 | 919 | 0.0460-92.0 |
| Retina | 200-018 | 30 | 548 | 0.0460-92.0 |
| Retina | 200-018 | 60 +/− 1 | 52.3 | 0.0460-92.0 |
| Retina | 200-018 | 60 +/− 1 | 159 | 0.0460-92.0 |
| Retina | 200-018 | 60 +/− 1 | 12.4 | 0.0460-92.0 |
| Retina | 200-018 | 60 +/− 1 | 259 | 0.0460-92.0 |
| Retina | 200-018 | 90 +/− 1 | 1020 | 0.460-2300 |
| Retina | 200-018 | 90 +/− 1 | 1110 | 0.460-2300 |
| Retina | 200-018 | 90 +/− 1 | 39.9 | 0.460-2300 |
| Retina | 200-018 | 90 +/− 1 | 120 | 0.460-2300 |
| Retina | 200-018 | 121 +/− 1 | 622 | 0.460-2300 |
| Retina | 200-018 | 121 +/− 1 | 6610 | 0.460-2300 |
| Retina | 200-018 | 121 +/− 1 | 0.0 | 0.460-2300 |
| Retina | 200-018 | 121 +/− 1 | 59.3 | 0.460-2300 |
| Retina | 200-018 | 150 +/− 2 | 46.3 | 0.460-2300 |
| Retina | 200-018 | 150 +/− 2 | 0.00 | 0.460-2300 |
| Retina | 200-018 | 180 +/− 2 | 294 | 0.460-2300 |
| Retina | 200-018 | 180 +/− 2 | 185 | 0.460-2300 |
| Retina | 200-018 | 180 +/− 2 | 11.9 | 0.460-2300 |
| Retina | 200-018 | 180 +/− 2 | 5.57 | 0.460-2300 |

### APPENDIX B

Compounds: 1 Ac-Phe-[Orn-Pro-cha-Trp-Phe] 2 Ac-Phe-[Orn-Hyp-cha-Trp-Phe] 3 HOCH.sub.2(CHOH).sub.4-C.dbd.N—O—CH.sub.2-CO-Phe-[Orn-Pro-cha-Trp-Nle]-4 X-Phe-[Orn-Pro-cha-Trp-Nle]; X=2-acetamido-1-methyl-glucuronyl 5 Ac-Phe-[Orn-Hyp(COCH.sub.2OCH.sub.2CH.sub.2OCH.sub.2CH.sub.2OCH.sub.3)-c-ha-Trp-Nle] 6 Ac-Phe-[Orn-Hyp(CONH—CH.sub.2CH(OH)—CH.sub.20H)-cha-Trp-Nle] 20 Ac-Phe-[Orn-Pro-cha-Trp-Ecr] 28 Ac-Phe-[Orn-Pro-cha-Trp-Nle] 29 Ac-Phe-[Orn-Pro-cha-Trp-Met] 31 Ac-Phe-[Orn-Pro-cha-Trp-Nva] 32 Ac-Phe-[Orn-Pro-cha-Trp-Hle] 33 Ac-Phe-[Orn-Pro-cha-Trp-Eaf] 34 Ac-Phe-[Orn-Pro-cha-Trp-Ebd] 35 Ac-Phe-[Orn-Pro-cha-Trp-Eag] 36 Ac-Phe-[Orn-Pro-cha-Trp-Pmf] 37 Ac-Phe-[Orn-Pro-cha-Trp-2Ni] 38 Ac-Phe-[Orn-Pro-cha-Trp-Thi] 41 Ph-CH.sub.2-CH.sub.2-CO-[Orn-Pro-cha-Trp-Nle] 42 H-Phe-[Orn-Pro-cha-Trp-Nle] 43 Ac-Lys-Phe-[Orn-Pro-cha-Trp-Nle] 44 H-Phe-[Orn-Ser-cha-Trp-Nle] 51 Ac-Phe-Orn-Pro-cha-Trp-Phe-NH.sub.2 52 Ac-Phe-Orn-Aze-cha-Bta-Phe-NH.sub.2 53 Ac-Phe-Orn-Pro-cha-Bta-2Ni—NH.sub.2 54 Ac-Phe-Orn-Pro-cha-Bta-Cha-NH.sub.2 55 Ac-Phe-Orn-Pip-cha-Trp-Phe-NH.sub.2 56 Ph-CH.sub.2-[Orn-Pro-cha-Trp-Nle] 57 Ph-CH.sub.2-[Orn-Pro-cha-Trp-Phe] 58 Ac-Phe-[Orn-Pro-cha-Trp-1Ni] 59 Ph-CH(OH)—CH.sub.2-CO-[Orn-Pro-cha-Trp-Nle] 61 Ac-Phe-Orn-Pro-cha-Trp-Phe-NH.sub.2 62 Ac-Phe-Orn-Pro-cha-Bta-Phe-NH.sub.2 64 Ac-Phe-Orn-Pro-cha-Trp-2Ni—NH.sub.2 65 Ac-Phe-Orn-Pro-cha-Trp-Cha-NH.sub.2 66 Ac-Thi-Orn-Aze-cha-Bta-Phe-NH.sub.2 67 Ac-Thi-Orn-Pip-cha-Bta-Phe-NH.sub.2 68 Ac-Phe-Orn-Pro-cha-Trp-Eap-NH.sub.2 69 Me.sub.2-Phe-Orn-Pro-cha-Trp-Phe-NH.sub.2 70 Ph.sub.2-CH—CH.sub.2-CO-Orn-Pro-cha-Trp-Phe-NH.sub.2 71 Ac-Ebw-Orn-Pro-cha-Trp-Phe-NH.sub.2 72 Ac-Phe-Orn-Pro-cha-Trp-NH—CH.sub.2-CH.sub.2-Ph 73 Ac-Phe-Orn-Aze-cha-Bta-NH—CH.sub.2-CH.sub.2-Ph 74 H-Phe-Orn-Pro-cha-Trp-Phe-NH.sub.2 75 H-Me-Phe-Orn-Pro-cha-Trp-Phe-NH.sub.2 76 Bu-NH—CO-Phe-Orn-Pro-cha-Trp-Phe-NH.sub.2 77 Ac-Thi-Orn-Pro-cha-Trp-Phe-NH.sub.2 78 Ac-Ebw-Orn-Pro-cha-Trp-Phe-NH.sub.2 79 Ac-Phe-Orn-Ala-cha-Trp-Phe-NH.sub.2 80 Ac-Phe-Orn-Pro-cha-Trp-Thi-NH.sub.2 81 Ac-Phe-Orn-Aze-cha-Pcf-Phe-NH.sub.2 82 Ac-Phe-Orn(Ac)-Pro-cha-Trp-Phe-NH.sub.2 83 Ac-Phe-Orn-Aze-cha-Trp-Phe-NH.sub.2 84 Ac-Phe-Trp-Pro-cha-Trp-Phe-NH.sub.2 85 Ph-NH—CO-Phe-Orn-Pro-cha-Trp-Phe-NH.sub.2 86 Bu-O—CO-Phe-Orn-Pro-cha-Trp-Phe-NH.sub.2 87 Ac-Phe-Lys-Pro-cha-Trp-Phe-NH.sub.2 88 Ac-Phe-Arg-Pro-cha-Trp-Phe-NH.sub.2 89 Ac-Phe-Gln-Pro-cha-Trp-Phe-NH.sub.2 92 Ac-Phe-Orn-Pip-cha-Trp-Phe-NH.sub.2 93 Ac-Phe-Orn-Hyp-cha-Trp-Phe-NH.sub.2 94 Ac-Phe-Orn-Pro-cha-Trp-1Ni—NH.sub.2 95 Ac-Phe-Orn-Aze-cha-Bta-Phe-NH-Me 96 CH.sub.3-SO.sub.2-Phe-Orn-Aze-cha-Bta-Phe-NH.sub.2 99 Ac-Phe-Orn-Aze-cha-Pff-Phe-NH.sub.2 100 Ac-Phe-Orn-Aze-cha-Mcf-Phe-NH.sub.2 101 Ac-Phe-Orn(Ac)-Aze-cha-Bta-Phe-NH.sub.2 102 Ac-Ebw-Orn-Pro-cha-Trp-Phe-NH.sub.2 103 Ac-Phe-Trp-Pro-cha-Trp-Phe-NH.sub.2 104 Ac-Phe-Arg-Pro-cha-Trp-Phe-NH.sub.2 105 Ac-Phe-Orn-Pip-cha-Trp-Phe-NH.sub.2 106 3PP-Orn-Aze-cha-Bta-Phe-NH.sub.2 107 Ac-Phe-Orn-Tic-cha-Trp-Phe-NH.sub.2 108 Ac-Phe-Orn-Ser-cha-Trp-Phe-NH.sub.2 109 Ac-Phe-Orn-Pro-chg-Trp-Phe-NH.sub.2 110 Ac-Phe-Orn-Pro-hch-Trp-Phe-NH.sub.2 111 Ac-Phe-Orn-Pro-cha-Trp-Phg-NH.sub.2 112 Ac-Phe-Bta-Aze-cha-Bta-Phe-NH.sub.2 113 Ac-Phe-Trp-Pro-cha-Bta-Phe-NH.sub.2 115 Ac-Phe-Orn-Pip-cha-Trp-Phe-OH 116 Ac-Phe-Orn-Tic-cha-Trp-Phe-OH 117 Ac-Phe-Orn-Ser-cha-Trp-Phe-OH 118 Ac-Phe-Orn-Pro-chg-Trp-Phe-OH 119 Ac-Phe-Eec-Pro-cha-Bta-Phe-NH.sub.2 120 Ac-Phe-Nle-Pro-cha-Bta-Phe-NH.sub.2 121 Ac-Phe-Har-Pro-cha-Bta-Phe-NH.sub.2 122 Ac-Phe-Arg-Pro-cha-Bta-Phe-NH.sub.2 123 Ac-Phe-Cys(Acm)-Pro-cha-Bta-Phe-NH.sub.2 124 Ac-Phe-Mpa-Pro-cha-Bta-Phe-NH.sub.2 125 Ac-Eby-Orn-Pro-cha-Bta-Phe-NH.sub.2 126 Ac-Phg-Orn-Pro-cha-Bta-Phe-NH.sub.2 127 Ac-Phe-Paf-Pro-cha-Bta-Phe-NH.sub.2 128 H.sub.2N—CO-Phe-Orn-Pro-cha-Bta-Phe-NH.sub.2 129 Me-O—CO-Phe-Orn-Pro-cha-Bta-Phe-NH.sub.2 130 (—CO—CH.sub.2-NH—CO—)-Phe-Orn-Pro-cha-Bta-Phe-NH.sub.2 132 Ac-Phe-Orn-Pro-hch-Trp-Phe-OH 133 (—CO—CH.sub.2-CH.sub.2-CO—)-Phe-Orn-Pro-cha-Bta-Phe-NH.sub.2 134.sup.tBu-CO-Phe-Orn-Pro-cha-Bta-Phe-NH.sub.2 135 Ac-Lys-Phe-Orn-Aze-cha-Bta-Phe-NH.sub.2 136 Ac-Gly-Phe-Orn-Aze-cha-Bta-Phe-NH.sub.2 137 Ac-Arg-Phe-Orn-Aze-cha-Bta-Phe-NH.sub.2 138 Ac-His-Phe-Orn-Aze-cha-Bta-Phe-NH.sub.2 139 Ac-Ser-Phe-Orn-Aze-cha-Bta-Phe-NH.sub.2 140 Ac-Guf-Phe-Orn-Aze-cha-Bta-Phe-NH.sub.2 141 Ac-Dab-Phe-Orn-Aze-cha-Bta-Phe-NH.sub.2 142 FH.sub.2C—CO-Phe-Orn-Pro-cha-Bta-Phe-NH.sub.2 143 Ac-Phe-Orn(Et.sub.2)-Pro-cha-Trp-Phe-NH.sub.2 144 Ac-Phe-[Orn-Hyp-cha-Trp-Nle] 145 3PP-[Orn-Hyp-cha-Trp-Nle] 146 Ac-Phe-[Orn-Pro-cha-Trp-Tyr] 147 Ac-Phe-[Orn-Pro-omf-Trp-Nle] 149 Ac-Phe-Orn-Pro-hle-Bta-Phe-NH.sub.2 150 Ac-Phe-Arg(CH.sub.2-CH.sub.2)-Pro-cha-Bta-Phe-NH.sub.2 151 Ac-Ala-Phe-Orn-Aze-cha-Bta-Phe-NH2 152 Ac-Arg-Phe-Orn-Aze-cha-Bta-Phe-NH2 153 Ac-Cit-Phe-Orn-Aze-cha-Bta-Phe-NH2 154 Ac-Gly-Phe-Orn-Aze-cha-Bta-Phe-NH2 155 Ac-Gly-Phe-Orn-Aze-chg-Bta-Phe-NH2 156 Ac-Gly-Phe-Orn-Aze-hch-Bta-Phe-NH2 157 Ac-Gly-Thi-Orn-Aze-cha-Bta-Phe-NH2 158 Ac-His-Phe-Orn-Aze-cha-Bta-Phe-NH2 159 Ac-Hyp-Phe-Orn-Aze-cha-Bta-Phe-NH2 160 Ac-Lys-Phe-Orn-Aze-cha-Bta-Phe-NH2 161 Ac-Mff-Orn-Pro-cha-Bta-Phe-NH2 162 Ac-Mff-Orn-Pro-hle-Bta-Phe-NH2 163 Ac-Mff-Orn-Pro-hle-Mcf-Mff-NH2 164 Ac-Mmy-Orn-Pro-hle-Pff-Phe-NH2 165 Ac-NMF-Orn-Pro-cha-Bta-Phe-NH2 166 Ac-Off-Orn-Pro-cha-Bta-Phe-NH2 167 Ac-Off-Orn-Pro-hle-Bta-Phe-NH2 168 Ac-Orn-Phe-Orn-Aze-cha-Bta-Phe-NH2 169 Ac-Pff-Orn-Pro-cha-Bta-Phe-NH2 170 Ac-Pff-Orn-Pro-hle-Bta-Phe-NH2 171 Ac-Pff-Orn-Pro-hle-Mcf-Pff-NH2 172 Ac-Phe-[Cys-Pro-cha-Bta-Phe-Cys]-NH2 173 Ac-Phe-[Orn-Asn-cha-Trp-Nle] 174 Ac-Phe-[Orn-Aze-cha-Trp-Nle] 175 Ac-Phe-[Orn-Chy-cha-Trp-Nle] 176 Ac-Phe-[Orn-HyA-cha-Trp-Phe] 177 Ac-Phe-[Orn-Hyp-hle-Bta-Phe] 178 Ac-Phe-[Orn-Hyp-hle-Mcf-Phe] 179 Ac-Phe-[Orn-Hyp-hle-Pff-Nle] 180 Ac-Phe-[Orn-Hyp-hle-Pff-Phe] 181 Ac-Phe-[Orn-Hyp-hle-Trp-Phe] 182 Ac-Phe-[Orn-Hyp-Mmf-Trp-Nle] 183 Ac-Phe-[Orn-Hyp-Mmf-Trp-Phe] 184 Ac-Phe-[Orn-NMD-cha-Trp-Nle] 185 Ac-Phe-[Orn-Pip-hle-Bta-Phe] 186 Ac-Phe-[Orn-Pro-cha-Pff-Nle] 187 Ac-Phe-[Orn-Pro-cha-Pff-Phe] 188 Ac-Phe-[Orn-Pro-cha-Trp-1Ni] 189 Ac-Phe-[Orn-Pro-cha-Trp-Cha] 190 Ac-Phe-[Orn-Pro-cha-Trp-Chg] 192 Ac-Phe-[Orn-Pro-cha-Trp-Ecr] 193 Ac-Phe-[Orn-Pro-cha-Trp-Leu] 194 Ac-Phe-[Orn-Pro-cha-Trp-nle] 195 Ac-Phe-[Orn-Pro-cha-Trp-Phe] 196 Ac-Phe-[Orn-Pro-hle-Bta-Nle] 197 Ac-Phe-[Orn-Pro-hle-Bta-Phe] 198 Ac-Phe-[Orn-Pro-hle-Pff-Phe] 199 Ac-Phe-[Orn-Pro-hle-Trp-Nle] 200 Ac-Phe-[Orn-Ser-cha-Trp-Nle] 201 Ac-Phe-[Orn-Ser-cha-Trp-Nle] 202 Ac-Phe-[Orn-Ser-hle-Trp-Nle] 203 Ac-Phe-[Orn-Thr-cha-Trp-Nle] 204 Ac-Phe-[Orn-Tic-cha-Trp-Nle] 205 Ac-Phe-[Orn-Tic-cha-Trp-Nle] 206 Ac-Phe-Ala-Pro-cha-Bta-Phe-NH2 207 Ac-Phe-Arg-Pro-hle-Bta-Phe-NH2 208 Ac-Phe-Arg-Pro-hle-Mcf-Phe-NH2 209 Ac-Phe-Cit-Hyp-hle-Bta-Phe-NH2 210 Ac-Phe-Cit-Pro-cha-Bta-Phe-NH2 211 Ac-Phe-Cit-Pro-hle-Bta-Phe-NH2 212 Ac-Phe-Cit-Ser-hle-Bta-Phe-NH2 213 Ac-Phe-Dab-Aze-cha-Bta-Phe-NH2 214 Ac-Phe-Dab-Aze-hle-Bta-Phe-NH2 215 Ac-Phe-Dab-Pro-cha-Bta-Phe-NH2 216 Ac-Phe-Dap-Pro-cha-Bta-Phe-NH2 217 Ac-Phe-Ech-Pro-cha-Bta-Phe-NH2 218 Ac-Phe-Eep-Pro-cha-Bta-Phe-NH2 219 Ac-Phe-Fcn-Aze-cha-Bta-Phe-NH2 220 Ac-Phe-Fcn-Pro-cha-Bta-Phe-NH2 221 Ac-Phe-Fco-Pro-cha-Bta-Phe-NH2 222 Ac-Phe-Fco-Pro-cha-Bta-Phe-NH2 223 Ac-Phe-Fcp-Aze-cha-Bta-Phe-NH2 224 Ac-Phe-Ffa-Aze-cha-Bta-Phe-NH2 225 Ac-Phe-Ffa-Pro-cha-Bta-Phe-NH2 226 Ac-Phe-Ffa-Pro-hle-Bta-Phe-NH2 227 Ac-Phe-G23-Pro-cha-Bta-Phe-NH2 228 Ac-Phe-Guf-Pro-cha-Bta-Phe-NH2 229 Ac-Phe-Har-Aze-cha-Bta-Phe-NH2 230 Ac-Phe-His-Pro-cha-Bta-Phe-NH2 231 Ac-Phe-L22-Pro-cha-Bta-Phe-NH2 232 Ac-Phe-OrA-Pro-cha-Bta-Phe-NH2 233 Ac-Phe-OrE-Pro-cha-Bta-Phe-NH2 234 Ac-Phe-Orn-Aze-hle-Bta-Phe-NH2 235 Ac-Phe-Orn-Chy-cha-Bta-Phe-NH2 236 Ac-Phe-Orn-Chy-hle-Pff-Phe-NH2 237 Ac-Phe-Orn-G24-cha-Bta-Phe-NH2 238 Ac-Phe-Orn-G25-cha-Bta-Phe-NH2 239 Ac-Phe-Orn-G26-cha-Bta-Phe-NH2 240 Ac-Phe-Orn-G27-cha-Bta-Phe-NH2 241 Ac-Phe-Orn-G30-cha-Bta-Phe-NH2 242 Ac-Phe-Orn-G31-cha-Bta-Phe-NH2 243 Ac-Phe-Orn-Hse-cha-Bta-Phe-NH2 244 Ac-Phe-Orn-Hyp-hle-Bta-Phe-NH2 245 Ac-Phe-Orn-Hyp-hle-Pff-Phe-NH2 246 Ac-Phe-Orn-NMA-cha-Bta-Phe-NH2 247 Ac-Phe-Orn-NMS-cha-Bta-Phe-NH2 248 Ac-Phe-Orn-Pro-cha-1Ni-Phe-NH2 249 Ac-Phe-Orn-Pro-cha-Bta-1Ni—NH2 250 Ac-Phe-Orn-Pro-cha-Bta-Bhf-NH2 251 Ac-Phe-Orn-Pro-cha-Bta-Dff-NH2 252 Ac-Phe-Orn-Pro-cha-Bta-Eaa-NH2 253 Ac-Phe-Orn-Pro-cha-Bta-L19 254 Ac-Phe-Orn-Pro-cha-Bta-Mcf-NH2 255 Ac-Phe-Orn-Pro-cha-Bta-Mff-NH2 256 Ac-Phe-Orn-Pro-cha-Bta-NH—CH(CH2OH)—CH2-Ph 257 Ac-Phe-Orn-Pro-Cha-Bta-NH-NBn-CO—NH2 258 Ac-Phe-Orn-Pro-cha-Bta-Opa-NH2 259 Ac-Phe-Orn-Pro-cha-Bta-Pcf-NH2 260 Ac-Phe-Orn-Pro-cha-Bta-Pmf-NH2 261 Ac-Phe-Orn-Pro-cha-Bta-Thi-NH2 262 Ac-Phe-Orn-Pro-cha-Otf-Phe-NH2 263 Ac-Phe-Orn-Pro-ctb-Bta-Phe-NH2 264 Ac-Phe-Orn-Pro-ctb-Eaa-Phe-NH2 265 Ac-Phe-Orn-Pro-ctb-Mcf-Phe-NH2 266 Ac-Phe-Orn-Pro-ctb-Pff-Phe-NH2 267 Ac-Phe-Orn-Pro-hch-Trp-Phe-OH 268 Ac-Phe-Orn-Pro-hle-1Ni-Phe-NH2 269 Ac-Phe-Orn-Pro-hle-6FW-Phe-NH2 270 Ac-Phe-Orn-Pro-hle-Bta-1Ni—NH2 271 Ac-Phe-Orn-Pro-hle-Bta-2Ni—NH2 272 Ac-Phe-Orn-Pro-hle-Bta-5Ff-NH2 273 Ac-Phe-Orn-Pro-hle-Bta-Aic-NH2 274 Ac-Phe-Orn-Pro-hle-Bta-Cha-NH2 275 Ac-Phe-Orn-Pro-hle-Bta-Chg-NH2 276 Ac-Phe-Orn-Pro-hle-Bta-Eaa-NH2 277 Ac-Phe-Orn-Pro-hle-Bta-Egy-NH2

278 Ac-Phe-Orn-Pro-hle-Bta-Pcf-NH2 279 Ac-Phe-Orn-Pro-hle-Bta-Pff-NH2 280 Ac-Phe-Orn-Pro-hle-Bta-Phe-NH2 281 Ac-Phe-Orn-Pro-hle-Bta-phe-OH 282 Ac-Phe-Orn-Pro-hle-Bta-Tyr-NH2 283 Ac-Phe-Orn-Pro-hle-Dff-Phe-NH2 284 Ac-Phe-Orn-Pro-hle-Eaa-Phe-NH2 285 Ac-Phe-Orn-Pro-hle-Egc-Phe-NH2 286 Ac-Phe-Orn-Pro-hle-Egy-Phe-NH2 287 Ac-Phe-Orn-Pro-hle-Egz-Phe-NH2 288 Ac-Phe-Orn-Pro-hle-Mcf-2Ni—NH2 289 Ac-Phe-Orn-Pro-hle-Mcf-Cha-NH2 290 Ac-Phe-Orn-Pro-hle-Mcf-Pff-NH2 291 Ac-Phe-Orn-Pro-hle-Mcf-Phe-NH2 292 Ac-Phe-Orn-Pro-hle-Mff-Phe-NH2 293 Ac-Phe-Orn-Pro-hle-Mmy-Phe-NH2 294 Ac-Phe-Orn-Pro-hle-Ocf-Phe-NH2 295 Ac-Phe-Orn-Pro-hle-Off-Phe-NH2 296 Ac-Phe-Orn-Pro-hle-Otf-Phe-NH2 297 Ac-Phe-Orn-Pro-hle-Pff-2Ni—NH2 298 Ac-Phe-Orn-Pro-hle-Pff-Cha-NH2 299 Ac-Phe-Orn-Pro-hle-Pff-Eaa-NH2 300 Ac-Phe-Orn-Pro-hle-Pff-Mmy-NH2 301 Ac-Phe-Orn-Pro-hle-Pff-Pff-NH2 302 Ac-Phe-Orn-Pro-hle-Pff-Phe-NH2 304 Ac-Phe-Orn-Pro-hle-Phe-Phe-NH2 305 Ac-Phe-Orn-Pro-hle-Tff-Phe-NH2 306 Ac-Phe-Orn-Pro-hle-Trp-Phe-NH2 307 Ac-Phe-Orn-Pro-ile-Trp-Phe-NH2 308 Ac-Phe-Orn-Pro-omf-Bta-Phe-NH2 309 Ac-Phe-Orn-Ser-cha-Bta-Phe-NH2 310 Ac-Ser-Phe-Orn-Aze-cha-Bta-Phe-NH2 311 Ac-Thi-[Orn-Pro-hle-Bta-Phe] 312 Ac-Thi-Orn-Pro-cha-Bta-Phe-NH2 313 Ac-Thi-Orn-Pro-cha-Bta-Thi-NH2 314 Ac-Thr-Phe-Orn-Aze-cha-Bta-Phe-NH2 315 Bzl-[Orn-Pro-cha-Bta-Nle] 316 CH3CH2CO-Phe-Orn-Pro-cha-Bta-Phe-NH2 317 Def-[Orn-Ser-hle-Trp-Nle] 318 Eby-Phe-[Orn-Hyp-cha-Trp-Phe] 319 Eth-Phe-[Orn-Pro-hle-Pff-Nle] 320 FAc-Phe-Fib-Aze-cha-Bta-Phe-NH2 321 FAc-Phe-Orn-Aze-cha-Bta-Phe-NH2 322 FAc-Phe-Orn-Pro-cha-Bta-Phe-NH2 323 Fai-Phe-[Orn-Hyp-cha-Trp-Phe] 324 Faz-Orn-Pro-cha-Bta-Phe-NH2 325 Fbi-Phe-[Orn-Pro-cha-Trp-Nle] 326 Fbn-Phe-[Orn-Hyp-cha-Trp-Phe] 327 Fbn-Phe-[Orn-Pro-cha-Trp-Nle] 328 Fbn-Phe-[Orn-Pro-cha-Trp-Nle] 329 Fbn-Phe-Cit-Pro-hle-Bta-Phe-NH2 330 Fbo-Phe-[Orn-Pro-cha-Trp-Nle] 331 Fbp-[Orn-Pro-cha-Trp-Nle] 332 Fci-[Phe-Orn-Hyp-cha-Trp-Phe] 333 Fck-[Phe-Orn-Pro-cha-Trp-Nle] 334 Fck-Phe-[Orn-Pro-cha-Trp-Nle] 335 Fha-Phe-[Orn-Hyp-cha-Trp-Phe] 336 Fhb-[Phe-Orn-Hyp-cha-Trp-Phe] 337 Fhi-Phe-[Orn-Hyp-cha-Trp-Phe] 338 Fhu-Phe-[Orn-Pro-hle-Pff-Nle] 339 Fhu-Phe-Orn-Pro-cha-Bta-Phe-NH2 340 Fid-Phe-Orn-Pro-cha-Bta-Phe-NH2 341 H-Amf-[Orn-Aze-hle-Pff-Nle] 342 H-Bal-Phe-[Orn-Hyp-hle-Trp-Nle] 343 H-Bal-Phe-[Orn-Pro-hle-Pff-Nle] 344 H-Eby-[Orn-Hyp-hle-Trp-Nle] 345 H-Gly-Phe-Orn-Pro-cha-Bta-Phe-NH2 346 H-Nip-Phe-Cit-Pro-hle-Bta-Phe-NH2 347 Hoo-Phe-[Orn-Hyp-hle-Pff-Nle] 348 Hoo-Phe-Cit-Pro-hle-Pff-Phe-NH2 349 Hoo-Phe-Orn-Hyp-hle-Pff-Phe-NH2 350 Hoo-Phe-Orn-Pro-hle-Bta-Phe-NH2 351 Hoo-Phe-Orn-Pro-hle-Mcf-Phe-NH2 352 Hoo-Phe-Orn-Pro-hle-Pff-Phe-NH2 353 H-Phe-[Lys-Hyp-hle-Pff-Nle] 354 H-Phe-[Orn-Hym-hle-Mcf-Nle] 355 H-Phe-[Orn-Hym-hle-Pff-Phe] 356 H-Phe-[Orn-Hyp-cha-Trp-Nle] 357 H-Phe-[Orn-Hyp-cha-Trp-Phe] 358 H-Phe-[Orn-Hyp-ctb-Pff-Nle] 359 H-Phe-[Orn-Hyp-ctb-Trp-Nle] 360 H-Phe-[Orn-Hyp-ctb-Trp-Phe] 361H-Phe-[Orn-Hyp-hle-Mcf-Leu] 362 H-Phe-[Orn-Hyp-hle-Pff-Chg] 363 H-Phe-[Orn-Hyp-hle-Pff-Hle] 364 H-Phe-[Orn-Hyp-hle-Pff-Leu] 365 H-Phe-[Orn-Hyp-hle-Pff-Nle] 366 H-Phe-[Orn-Hyp-hle-Pff-Phe] 367 H-Phe-[Orn-Hyp-hle-Trp-Hle] 368 H-Phe-[Orn-Hyp-hle-Trp-Leu] 369 H-Phe-[Orn-Hyp-hle-Trp-Nle] 370 H-Phe-[Orn-Hyp-hle-Trp-Nva] 371 H-Phe-[Orn-Hyp-hle-Trp-Phe] 372 H-Phe-[Orn-NMS-cha-Trp-Nle] 373 H-Phe-[Orn-NMS-hle-Pff-Phe] 374 H-Phe-[Orn-Pro-cha-Pff-Nle] 375 H-Phe-[Orn-Pro-cha-Pff-Phe] 376 H-Phe-[Orn-Pro-cha-Trp-Nle] 377 H-Phe-[Orn-Pro-hle-Mcf-Phe] 378 H-Phe-[Orn-Pro-hle-Ocf-Phe] 379 H-Phe-[Orn-Pro-hle-Pff-Nle] 380 H-Phe-[Orn-Pro-hle-Pff-Phe] 381 H-Phe-[Orn-Pro-hle-Trp-Nle] 382 H-Phe-[Orn-Ser-cha-Trp-Nle] 383 H-Phe-[Orn-Ser-cha-Trp-Phe] 384 H-Phe-[Orn-Ser-hle-Eaa-Nle] 385 H-Phe-[Orn-Ser-hle-Mcf-Leu] 386 H-Phe-[Orn-Ser-hle-Ocf-Nle] 387 H-Phe-[Orn-Ser-hle-Pff-Leu] 388 H-Phe-[Orn-Ser-hle-Pff-Nle] 389 H-Phe-[Orn-Ser-hle-Pff-Phe] 390 H-Phe-[Orn-Ser-hle-Trp-Nle] 391 H-Phe-Cit-Pro-hle-Bta-Phe-NH2 392 Ohf-[Orn-Hyp-hle-Trp-Nle] 393 Tmg-Phe-[Orn-Hyp-cha-Trp-Phe].

### APPENDIX C

In certain embodiments, compounds and antagonists according to the present invention are the following cyclic compounds. Compound 1 Ac-Phe-[Orn-Pro-cha-Trp-Phe] 2 Ac-Phe-[Orn-Hyp-cha-Trp-Phe] 3 HOCH2(CHOH)4-C.dbd.N—O—CH2-CO-Phe-[Orn-Pro-cha-Trp-Nle]-4 X-Phe-[Orn-Pro-cha-Trp-Nle]; X=2-Acetamido-1-Methyl-Glucuronyl 5 Ac-Phe-[Orn-Hyp(COCH2OCH2CH2OCH2CH2OCH3)-cha-Trp-Nle] 6 Ac-Phe-[Orn-Hyp(CONH—CH2CH(OH)—CH2OH)-cha-Trp-Nle] 20 Ac-Phe-[Orn-Pro-cha-Trp-Ecr] 28 Ac-Phe-[Orn-Pro-cha-Trp-Nle] 29 Ac-Phe-[Orn-Pro-cha-Trp-Met] 31 Ac-Phe-[Orn-Pro-cha-Trp-Nva] 32 Ac-Phe-[Orn-Pro-cha-Trp-Hle] 33 Ac-Phe-[Orn-Pro-cha-Trp-Eaf] 34 Ac-Phe-[Orn-Pro-cha-Trp-Ebd] 35 Ac-Phe-[Orn-Pro-cha-Trp-Eag] 36 Ac-Phe-[Orn-Pro-cha-Trp-Pmf] 37 Ac-Phe-[Orn-Pro-cha-Trp-2Ni] 38 Ac-Phe-[Orn-Pro-cha-Trp-Thi] 41 Ph-CH2-CH2-CO—[Orn-Pro-cha-Trp-Nle] 42 H-Phe-[Orn-Pro-cha-Trp-Nle] 43 Ac-Lys-Phe-[Orn-Pro-cha-Trp-Nle] 44 H-Phe-[Orn-Ser-cha-Trp-Nle] 56 Ph-CH2-[Orn-Pro-cha-Trp-Nle] 57 Ph-CH2-[Orn-Pro-cha-Trp-Phe] 58 Ac-Phe-[Orn-Pro-cha-Trp-1Ni] 59 Ph-CH(OH)—CH2-CO—[Orn-Pro-cha-Trp-Nle] 144 Ac-Phe-[Orn-Hyp-cha-Trp-Nle] 145 3PP-[Orn-Hyp-cha-Trp-Nle] 146 Ac-Phe-[Orn-Pro-cha-Trp-Tyr] 147 Ac-Phe-[Orn-Pro-omf-Trp-Nle] 172 Ac-Phe-[Cys-Pro-cha-Bta-Phe-Cys]-NH2 173 Ac-Phe-[Orn-Asn-cha-Trp-Nle] 174 Ac-Phe-[Orn-Aze-cha-Trp-Nle] 175 Ac-Phe-[Orn-Chy-cha-Trp-Nle] 176 Ac-Phe-[Orn-HyA-cha-Trp-Phe] 177 Ac-Phe-[Orn-Hyp-hle-Bta-Phe] 178 Ac-Phe-[Orn-Hyp-hle-Mcf-Phe] 179 Ac-Phe-[Orn-Hyp-hle-Pff-Nle] 180 Ac-Phe-[Orn-Hyp-hle-Pff-Phe] 181 Ac-Phe-[Orn-Hyp-hle-Trp-Phe] 182 Ac-Phe-[Orn-Hyp-Mmf-Trp-Nle] 183 Ac-Phe-[Orn-Hyp-Mmf-Trp-Phe] 184 Ac-Phe-[Orn-NMD-cha-Trp-Nle] 185 Ac-Phe-[Orn-Pip-hle-Bta-Phe] 186 Ac-Phe-[Orn-Pro-cha-Pff-Nle] 187 Ac-Phe-[Orn-Pro-cha-Pff-Phe] 188 Ac-Phe-[Orn-Pro-cha-Trp-1Ni] 189 Ac-Phe-[Orn-Pro-cha-Trp-Cha] 190 Ac-Phe-[Orn-Pro-cha-Trp-Chg] 192 Ac-Phe-[Orn-Pro-cha-Trp-Ecr] 193 Ac-Phe-[Orn-Pro-cha-Trp-Leu] 194 Ac-Phe-[Orn-Pro-cha-Trp-nle] 195 Ac-Phe-[Orn-Pro-cha-Trp-Phe] 196 Ac-Phe-[Orn-Pro-hle-Bta-Nle] 197 Ac-Phe-[Orn-Pro-hle-Bta-Phe] 198 Ac-Phe-[Orn-Pro-hle-Pff-Phe] 199 Ac-Phe-[Orn-Pro-hle-Trp-Nle] 200 Ac-Phe-[Orn-Ser-cha-Trp-Nle] 201 Ac-Phe-[Orn-Ser-cha-Trp-Nle] 202 Ac-Phe-[Orn-Ser-hle-Trp-Nle] 203 Ac-Phe-[Orn-Thr-cha-Trp-Nle] 204 Ac-Phe-[Orn-Tic-cha-Trp-Nle] 205 Ac-Phe-[Orn-Tic-cha-Trp-Nle] 311 Ac-Thi-[Orn-Pro-hle-Bta-Phe] 315 Bzl-[Orn-Pro-cha-Bta-Nle] 317 Def-[Orn-Ser-hle-Trp-Nle] 318 Eby-Phe-[Orn-Hyp-cha-Trp-Phe] 319 Eth-Phe-[Orn-Pro-hle-Pff-Nle] 323 Fai-Phe-[Orn-Hyp-cha-Trp-Phe] 325 Fbi-Phe-[Orn-Pro-cha-Trp-Nle] 326 Fbn-Phe-[Orn-Hyp-cha-Trp-Phe] 327 Fbn-Phe-[Orn-Pro-cha-Trp-Nle] 328 Fbn-Phe-[Orn-Pro-cha-Trp-Nle] 330 Fbo-Phe-[Orn-Pro-cha-Trp-Nle] 331 Fbp-[Orn-Pro-cha-Trp-Nle] 332 Fci-[Phe-Orn-Hyp-cha-Trp-Phe] 333 Fck-[Phe-Orn-Pro-cha-Trp-Nle] 334 Fck-Phe-[Orn-Pro-cha-Trp-Nle]335 Fha-Phe-[Orn-Hyp-cha-Trp-Phe] 336 Fhb-[Phe-Orn-Hyp-cha-Trp-Phe] 337 Fhi-Phe-[Orn-Hyp-cha-Trp-Phe] 338 Fhu-Phe-[Orn-Pro-hle-Pff-Nle] 341 H-Amf-[Orn-Aze-hle-Pff-Nle] 342 H-Bal-Phe-[Orn-Hyp-hle-Trp-Nle] 343 H-Bal-Phe-[Orn-Pro-hle-Pff-Nle] 344 H-Eby-[Orn-Hyp-hle-Trp-Nle] 347 Hoo-Phe-[Orn-Hyp-hle-Pff-Nle] 353 H-Phe-[Lys-Hyp-hle-Pff-Nle] 354 H-Phe-[Orn-Hym-hle-Mcf-Nle] 355 H-Phe-[Orn-Hym-hle-Pff-Phe] 356 H-Phe-[Orn-Hyp-cha-Trp-Nle] 357 H-Phe-[Orn-Hyp-cha-Trp-Phe] 358 H-Phe-[Orn-Hyp-ctb-Pff-Nle] 359 H-Phe-[Orn-Hyp-ctb-Trp-Nle] 360 H-Phe-[Orn-Hyp-ctb-Trp-Phe] 361 H-Phe-[Orn-Hyp-hle-Mcf-Leu] 362 H-Phe-[Orn-Hyp-hle-Pff-Chg] 363 H-Phe-[Orn-Hyp-hle-Pff-Hle] 364 H-Phe-[Orn-Hyp-hle-Pff-Leu] 365 H-Phe-[Orn-Hyp-hle-Pff-Nle] 366 H-Phe-[Orn-Hyp-hle-Pff-Phe] 367 H-Phe-[Orn-Hyp-hle-Trp-Hle] 368 H-Phe-[Orn-Hyp-hle-Trp-Leu] 369 H-Phe-[Orn-Hyp-hle-Trp-Nle] 370 H-Phe-[Orn-Hyp-hle-Trp-Nva] 371 H-Phe-[Orn-Hyp-hle-Trp-Phe] 372 H-Phe-[Orn-NMS-cha-Trp-Nle] 373 H-Phe-[Orn-NMS-hle-Pff-Phe] 374 H-Phe-[Orn-Pro-cha-Pff-Nle] 375 H-Phe-[Orn-Pro-cha-Pff-Phe] 376 H-Phe-[Orn-Pro-cha-Trp-Nle] 377 H-Phe-[Orn-Pro-hle-Mcf-Phe] 378 H-Phe-[Orn-Pro-hle-Ocf-Phe] 379 H-Phe-[Orn-Pro-hle-Pff-Nle] 380 H-Phe-[Orn-Pro-hle-Pff-Phe] 381 H-Phe-[Orn-Pro-hle-Trp-Nle] 382 H-Phe-[Orn-Ser-cha-Trp-Nle] 383 H-Phe-[Orn-Ser-cha-Trp-Phe] 384 H-Phe-[Orn-Ser-hle-Eaa-Nle] 385 H-Phe-[Orn-Ser-hle-Mcf-Leu] 386 H-Phe-[Orn-Ser-hle-Ocf-Nle] 387 H-Phe-[Orn-Ser-hle-Pff-Leu] 388 H-Phe-[Orn-Ser-hle-Pff-Nle] 389 H-Phe-[Orn-Ser-hle-Pff-Phe] 390 H-Phe-[Orn-Ser-hle-Trp-Nle] 392 Ohf-[Orn-Hyp-hle-Trp-Nle] 393 Tmg-Phe-[Orn-Hyp-cha-Trp-Phe]

### APPENDIX D

In certain embodiments, linear peptidic inhibitors according to the invention are compounds: 51 Ac-Phe-Orn-Pro-cha-Trp-Phe-NH2 52 Ac-Phe-Orn-Aze-cha-Bta-Phe-NH2 53 Ac-Phe-Orn-Pro-cha-Bta-2Ni—NH2 54 Ac-Phe-Orn-Pro-cha-Bta-Cha-NH2 55 Ac-Phe-Orn-Pip-cha-Trp-Phe-NH2 61 Ac-Phe-Orn-Pro-cha-Trp-Phe-NH2 62 Ac-Phe-Orn-Pro-cha-Bta-Phe-NH2 64 Ac-Phe-Orn-Pro-cha-Trp-2Ni—NH2 65 Ac-Phe-Orn-Pro-cha-Trp-Cha-NH2 66 Ac-Thi-Orn-Aze-cha-Bta-Phe-NH2 67 Ac-Thi-Orn-Pip-cha-Bta-Phe-NH2 68 Ac-Phe-Orn-Pro-cha-Trp-Eap-NH2 69 Me2-Phe-Orn-Pro-cha-Trp-Phe-NH2 70 Ph2-CH—CH2-CO-Orn-Pro-cha-Trp-Phe-NH2 71 Ac-Ebw-Orn-Pro-cha-Trp-Phe-NH2 72 Ac-Phe-Orn-Pro-cha-Trp-NH—CH2—CH2-Ph 73 Ac-Phe-Orn-Aze-cha-Bta-NH—CH2-CH2-Ph 74 H-Phe-Orn-Pro-cha-Trp-Phe-NH2 75 H-Me-Phe-Orn-Pro-cha-Trp-Phe-NH2 76 Bu-NH—CO-Phe-Orn-Pro-cha-Trp-Phe-NH2 77 Ac-Thi-Orn-Pro-cha-Trp-Phe-NH2 78 Ac-Ebw-Orn-Pro-cha-Trp-Phe-NH2 79 Ac-Phe-Orn-Ala-cha-Trp-Phe-NH2 80 Ac-Phe-Orn-Pro-cha-Trp-Thi-NH2 81 Ac-Phe-Orn-Aze-cha-Pcf-Phe-NH2 82 Ac-Phe-Orn(Ac)-Pro-cha-Trp-Phe-NH2 83 Ac-Phe-Orn-Aze-cha-Trp-Phe-NH2 84 Ac-Phe-Trp-Pro-cha-Trp-Phe-NH2 85 Ph-NH—CO-Phe-Orn-Pro-cha-Trp-Phe-NH2 86 Bu-O—CO-Phe-Orn-Pro-cha-Trp-Phe-NH2 87 Ac-Phe-Lys-Pro-cha-Trp-Phe-NH2 88 Ac-Phe-Arg-Pro-cha-Trp-Phe-NH2 89 Ac-Phe-Gln-Pro-cha-Trp-Phe-NH2 90 Ac-Phe-Ser-Pro-cha-Trp-Phe-NH2 91 Ac-Phe-Glu-Pro-cha-Trp-Phe-NH2 92 Ac-Phe-Orn-Pip-cha-Trp-Phe-NH2 93 Ac-Phe-Orn-Hyp-cha-Trp-Phe-NH2 94 Ac-Phe-Orn-Pro-cha-Trp-1Ni—NH2 95 Ac-Phe-Orn-Aze-cha-Bta-Phe-NH-Me 96 CH3-SO2-Phe-Orn-Aze-cha-Bta-Phe-NH2 99 Ac-Phe-Orn-Aze-cha-Pff-Phe-NH2 100 Ac-Phe-Orn-Aze-cha-Mcf-Phe-NH2 101 Ac-Phe-Orn(Ac)-Aze-cha-Bta-Phe-NH2 102 Ac-Ebw-Orn-Pro-cha-Trp-Phe-NH2 103 Ac-Phe-Trp-Pro-cha-Trp-Phe-NH2 104 Ac-Phe-Arg-Pro-cha-Trp-Phe-NH2 105 Ac-Phe-Orn-Pip-cha-Trp-Phe-NH2 106 3PP-Orn-Aze-cha-Bta-Phe-NH2 107 Ac-Phe-Orn-Tic-cha-Trp-Phe-NH2 108 Ac-Phe-Orn-Ser-cha-Trp-Phe-NH2 109 Ac-Phe-Orn-Pro-chg-Trp-Phe-NH2 110 Ac-Phe-Orn-Pro-hch-Trp-Phe-NH2 111 Ac-Phe-Orn-Pro-cha-Trp-Phg-NH2 112 Ac-Phe-Bta-Aze-cha-Bta-Phe-NH2 113 Ac-Phe-Trp-Pro-cha-Bta-Phe-NH2 115 Ac-Phe-Orn-Pip-cha-Trp-Phe-OH 116 Ac-Phe-Orn-Tic-cha-Trp-Phe-OH 117 Ac-Phe-Orn-Ser-cha-Trp-Phe-OH 118 Ac-Phe-Orn-Pro-chg-Trp-Phe-OH 119 Ac-Phe-Eec-Pro-cha-Bta-Phe-NH2 120 Ac-Phe-Nle-Pro-cha-Bta-Phe-NH2 121 Ac-Phe-Har-Pro-cha-Bta-Phe-NH2 122 Ac-Phe-Arg-Pro-cha-Bta-Phe-NH2 123 Ac-Phe-Cys(Acm)-Pro-cha-Bta-Phe-NH2 124 Ac-Phe-Mpa-Pro-cha-Bta-Phe-NH2 125 Ac-Eby-Orn-Pro-cha-Bta-Phe-NH2 126 Ac-Phg-Orn-Pro-cha-Bta-Phe-NH2 127 Ac-Phe-Paf-Pro-cha-Bta-Phe-NH2 128 H2N—CO-Phe-Orn-Pro-cha-Bta-Phe-NH2 129 Me-O—CO-Phe-Orn-Pro-cha-Bta-Phe-NH2 130 (—CO—CH2-NH—CO—)-Phe-Orn-Pro-cha-Bta-Phe-NH2 132 Ac-Phe-Orn-Pro-hch-Trp-Phe-OH 133 (—CO—CH2-CH2-CO—)-Phe-Orn-Pro-cha-Bta-Phe-NH2 134 tBu-CO-Phe-Orn-Pro-cha-Bta-Phe-NH2 135 Ac-Lys-Phe-Orn-Aze-cha-Bta-Phe-NH2 136 Ac-Gly-Phe-Orn-Aze-cha-Bta-Phe-NH2 137 Ac-Arg-Phe-Orn-Aze-cha-Bta-Phe-NH2 138 Ac-His-Phe-Orn-Aze-cha-Bta-Phe-NH2 139 Ac-Ser-Phe-Orn-Aze-cha-Bta-Phe-NH2 140 Ac-Guf-Phe-Orn-Aze-cha-Bta-Phe-NH2 141 Ac-Dab-Phe-Orn-Aze-cha-Bta-Phe-NH2 142 FH2C—CO-Phe-Orn-Pro-cha-Bta-Phe-NH2 143 Ac-Phe-Orn(Et2)-Pro-cha-Trp-Phe-NH2 148 Ac-Phe-N(nBu)-CH2-CO-Pro-cha-Trp-Phe-NH2 149 Ac-Phe-Orn-Pro-hle-Bta-Phe-NH2 150 Ac-Phe-Arg(CH2-CH2)-Pro-cha-Bta-Phe-NH2 151 Ac-Ala-Phe-Orn-Aze-cha-Bta-Phe-NH2 152 Ac-Arg-Phe-Orn-Aze-cha-Bta-Phe-NH2 153 Ac-Cit-Phe-Orn-Aze-cha-Bta-Phe-NH2 154 Ac-Gly-Phe-Orn-Aze-cha-Bta-Phe-NH2 155 Ac-Gly-Phe-Orn-Aze-chg-Bta-Phe-NH2 156 Ac-Gly-Phe-Orn-Aze-hch-Bta-Phe-NH2 157 Ac-Gly-Thi-Orn-Aze-cha-Bta-Phe-NH2 158 Ac-His-Phe-Orn-Aze-cha-Bta-Phe-NH2 159 Ac-Hyp-Phe-Orn-Aze-cha-Bta-Phe-NH2 160 Ac-Lys-Phe-Orn-Aze-cha-Bta-Phe-NH2 161 Ac-Mff-Orn-Pro-cha-Bta-Phe-NH2 162 Ac-Mff-Orn-Pro-hle-Bta-Phe-NH2 163 Ac-Mff-Orn-Pro-hle-Mcf-Mff-NH2 164 Ac-Mmy-Orn-Pro-hle-Pff-Phe-NH2 165 Ac-NMF-Orn-Pro-cha-Bta-Phe-NH2 166 Ac-Off-Orn-Pro-cha-Bta-Phe-NH2 167 Ac-Off-Orn-Pro-hle-Bta-Phe-NH2 168 Ac-Orn-Phe-Orn-Aze-cha-Bta-Phe-NH2 169 Ac-Pff-Orn-Pro-cha-Bta-Phe-NH2 170 Ac-Pff-Orn-Pro-hle-Bta-Phe-NH2 171 Ac-Pff-Orn-Pro-hle-Mcf-Pff-NH2 206 Ac-Phe-Ala-Pro-cha-Bta-Phe-NH2 207 Ac-Phe-Arg-Pro-hle-Bta-Phe-NH2 208 Ac-Phe-Arg-Pro-hle-Mcf-Phe-NH2 209 Ac-Phe-Cit-Hyp-hle-Bta-Phe-NH2 210 Ac-Phe-Cit-Pro-cha-Bta-Phe-NH2 211 Ac-Phe-Cit-Pro-hle-Bta-Phe-NH2 212 Ac-Phe-Cit-Ser-hle-Bta-Phe-NH2 213 Ac-Phe-Dab-Aze-cha-Bta-Phe-NH2 214 Ac-Phe-Dab-Aze-hle-Bta-Phe-NH2 215 Ac-Phe-Dab-Pro-cha-Bta-Phe-NH2 216 Ac-Phe-Dap-Pro-cha-Bta-Phe-NH2 217 Ac-Phe-Ech-Pro-cha-Bta-Phe-NH2 218 Ac-Phe-Eep-Pro-cha-Bta-Phe-NH2 219 Ac-Phe-Fcn-Aze-cha-Bta-Phe-NH2 220 Ac-Phe-Fcn-Pro-cha-Bta-Phe-NH2 221 Ac-Phe-Fco-Pro-cha-Bta-Phe-NH2 222 Ac-Phe-Fco-Pro-cha-Bta-Phe-NH2 223 Ac-Phe-Fcp-Aze-cha-Bta-Phe-NH2 224 Ac-Phe-Ffa-Aze-cha-Bta-Phe-NH2 225 Ac-Phe-Ffa-Pro-cha-Bta-Phe-NH2 226 Ac-Phe-Ffa-Pro-hle-Bta-Phe-NH2 227 Ac-Phe-G23-Pro-cha-Bta-Phe-NH2 228 Ac-Phe-Guf-Pro-cha-Bta-Phe-NH2 229 Ac-Phe-Har-Aze-cha-Bta-Phe-NH2 230 Ac-Phe-His-Pro-cha-Bta-Phe-NH2 231 Ac-Phe-L22-Pro-cha-Bta-Phe-NH2 232 Ac-Phe-OrA-Pro-cha-Bta-Phe-NH2 233 Ac-Phe-OrE-Pro-cha-Bta-Phe-NH2 234 Ac-Phe-Orn-Aze-hle-Bta-Phe-NH2 235 Ac-Phe-Orn-Chy-cha-Bta-Phe-NH2 236 Ac-Phe-Orn-Chy-hle-Pff-Phe-NH2 237 Ac-Phe-Orn-G24-cha-Bta-Phe-NH2 238 Ac-Phe-Orn-G25-cha-Bta-Phe-NH2 239 Ac-Phe-Orn-G26-cha-Bta-Phe-NH2 240 Ac-Phe-Orn-G27-cha-Bta-Phe-NH2 241 Ac-Phe-Orn-G30-cha-Bta-Phe-NH2 242 Ac-Phe-Orn-G31-cha-Bta-Phe-NH2 243 Ac-Phe-Orn-Hse-cha-Bta-Phe-NH2 244 Ac-Phe-Orn-Hyp-hle-Bta-Phe-NH2 245 Ac-Phe-Orn-Hyp-hle-Pff-Phe-NH2 246 Ac-Phe-Orn-NMA-cha-Bta-Phe-NH2 247 Ac-Phe-Orn-NMS-cha-Bta-Phe-NH2 248 Ac-Phe-Orn-Pro-cha-1Ni-Phe-NH2 249 Ac-Phe-Orn-Pro-cha-Bta-1N—NH2 250 Ac-Phe-Orn-Pro-cha-Bta-Bhf-NH2 251 Ac-Phe-Orn-Pro-cha-Bta-Dff-NH2 252 Ac-Phe-Orn-Pro-cha-Bta-Eaa-NH2 253 Ac-Phe-Orn-Pro-cha-Bta-L19 254 Ac-Phe-Orn-Pro-cha-Bta-Mcf-NH2 255 Ac-Phe-Orn-Pro-cha-Bta-Mff-NH2 256 Ac-Phe-Orn-Pro-cha-Bta-NH—CH(CH20H)—CH2-Ph 257 Ac-Phe-Orn-Pro-Cha-Bta-NH-NBn-CO—NH2 258 Ac-Phe-Orn-Pro-cha-Bta-Opa-NH2 259 Ac-Phe-Orn-Pro-cha-Bta-Pcf-NH2 260 Ac-Phe-Orn-Pro-cha-Bta-Pmf-NH2 261 Ac-Phe-Orn-Pro-cha-Bta-Thi-NH2 262 Ac-Phe-Orn-Pro-cha-Otf-Phe-NH2 263 Ac-Phe-Orn-Pro-ctb-Bta-Phe-NH2 264 Ac-Phe-Orn-Pro-ctb-Eaa-Phe-NH2 265 Ac-Phe-Orn-Pro-ctb-Mcf-Phe-NH2 266 Ac-Phe-Orn-Pro-ctb-Pff-Phe-NH2 267 Ac-Phe-Orn-Pro-hch-Trp-Phe-OH 268 Ac-Phe-Orn-Pro-hle-1Ni-Phe-NH2 269 Ac-Phe-Orn-Pro-hle-6FW-Phe-NH2 270 Ac-Phe-Orn-Pro-hle-Bta-1Ni—NH2 271 Ac-Phe-Orn-Pro-hle-Bta-2Ni—NH2 272 Ac-Phe-Orn-Pro-hle-Bta-5Ff-NH2 273 Ac-Phe-Orn-Pro-hle-Bta-Aic-NH2 274 Ac-Phe-Orn-Pro-hle-Bta-Cha-NH2 275 Ac-Phe-Orn-Pro-hle-Bta-Chg-NH2 276 Ac-Phe-Orn-Pro-hle-Bta-Eaa-NH2 277 Ac-Phe-Orn-Pro-hle-Bta-Egy-NH2 278 Ac-Phe-Orn-Pro-hle-Bta-Pcf-NH2 279 Ac-Phe-Orn-Pro-hle-Bta-Pff-NH2 280 Ac-Phe-Orn-Pro-hle-Bta-Phe-NH2 281 Ac-Phe-Orn-Pro-hle-Bta-phe-OH 282 Ac-Phe-Orn-Pro-hle-Bta-Tyr-NH2 283 Ac-Phe-Orn-Pro-hle-Dff-Phe-NH2 284 Ac-Phe-Orn-Pro-hle-Eaa-Phe-NH2 285 Ac-Phe-Orn-Pro-hle-Egc-Phe-NH2 286 Ac-Phe-Orn-Pro-hle-Egy-Phe-NH2 287 Ac-Phe-Orn-Pro-hle-Egz-Phe-NH2 288 Ac-Phe-Orn-Pro-hle-Mcf-2Ni—NH2 289 Ac-Phe-Orn-Pro-hle-Mcf-Cha-NH2 290 Ac-Phe-Orn-Pro-hle-Mcf-Pff-NH2 291 Ac-Phe-Orn-Pro-hle-Mcf-Phe-NH2 292 Ac-Phe-Orn-Pro-hle-Mff-Phe-NH2 293 Ac-Phe-Orn-Pro-hle-Mmy-Phe-NH2 294 Ac-Phe-Orn-Pro-hle-Ocf-Phe-NH2 295 Ac-Phe-Orn-Pro-hle-Off-Phe-NH2 296 Ac-Phe-Orn-Pro-hle-Otf-Phe-NH2 297 Ac-Phe-Orn-Pro-hle-Pff-2Ni—NH2 298 Ac-Phe-Orn-Pro-hle-Pff-Cha-NH2 299 Ac-Phe-Orn-Pro-hle-Pff-Eaa-NH2 300 Ac-Phe-Orn-Pro-hle-Pff-Mmy-NH2 301 Ac-Phe-Orn-Pro-hle-Pff-Pff-NH2 302 Ac-Phe-Orn-Pro-hle-Pff-Phe-NH2 304 Ac-Phe-Orn-Pro-hle-Phe-Phe-NH2 305 Ac-Phe-Orn-Pro-hle-Tff-Phe-NH2 306 Ac-Phe-Orn-Pro-hle-Trp-Phe-NH2 307 Ac-Phe-Orn-Pro-ile-Trp-Phe-NH2 308 Ac-Phe-Orn-Pro-omf-Bta-Phe-NH2 309 Ac-Phe-Orn-Ser-cha-Bta-Phe-NH2 310 Ac-Ser-Phe-Orn-Aze-cha-Bta-Phe-NH2 312 Ac-Thi-Orn-Pro-cha-Bta-Phe-NH2 313 Ac-Thi-Orn-Pro-cha-Bta-Thi-NH2 314 Ac-Thr-Phe-Orn-Aze-cha-Bta-Phe-NH2 316 CH3CH2CO-Phe-Orn-Pro-cha-Bta-Phe-NH2 320 FAc-Phe-Fib-Aze-cha-Bta-Phe-NH2 321 FAc-Phe-Orn-Aze-cha-Bta-Phe-NH2 322 FAc-Phe-Orn-Pro-cha-Bta-Phe-NH2 324 Faz-Orn-Pro-cha-Bta-Phe-NH2 329 Fbn-Phe-Cit-Pro-hle-Bta-Phe-NH2 339 Fhu-Phe-Orn-Pro-cha-Bta-Phe-NH2 340 Fid-Phe-Orn-Pro-cha-Bta-Phe-NH2 345 H-Gly-Phe-Orn-Pro-cha-Bta-Phe-NH2 346 H-Nip-Phe-Cit-Pro-hle-Bta-Phe-NH2 348 Hoo-Phe-Cit-Pro-hle-Pff-Phe-NH2 349 Hoo-Phe-Orn-Hyp-hle-Pff-Phe-NH2 350 Hoo-Phe-Orn-Pro-hle-Bta-Phe-NH2 351 Hoo-Phe-Orn-Pro-hle-Mcf-Phe-NH2 352 Hoo-Phe-Orn-Pro-hle-Pff-Phe-NH2 391 H-Phe-Cit-Pro-hle-Bta-Phe-NH2.

### APPENDIX E

In certain embodiments, the invention uses the following active agents: Compound: 1 Ac-Phe-[Orn-Pro-cha-Trp-Phe] 2 Ac-Phe-[Orn-Hyp-cha-Trp-Phe] 3 HOCH.sub.2(CHOH).sub.4-C.dbd.N—O—CH.sub.2-CO-Phe-[Orn-Pro-cha-Trp-Nle]-4 X-Phe-[Orn-Pro-cha-Trp-Nle]; X=2-acetamido-1-methyl-glucuronyl 5 Ac-Phe-[Orn-Hyp(COCH.sub.2OCH.sub.2CH.sub.2OCH.sub.2CH.sub.2OCH.sub.3)-c-ha-Trp-Nle] 6 Ac-Phe-[Orn-Hyp(CONH—CH.sub.2CH(OH)—CH.sub.20H)-cha-Trp-Nle] 20 Ac-Phe-[Orn-Pro-cha-Trp-Ecr] 28 Ac-Phe-[Orn-Pro-cha-Trp-Nle] 29 Ac-Phe-[Orn-Pro-cha-Trp-Met] 31 Ac-Phe-[Orn-Pro-cha-Trp-Nva] 32 Ac-Phe-[Orn-Pro-cha-Trp-Hle] 33 Ac-Phe-[Orn-Pro-cha-Trp-Eaf] 34 Ac-Phe-[Orn-Pro-cha-Trp-Ebd] 35 Ac-Phe-[Orn-Pro-cha-Trp-Eag] 36 Ac-Phe-[Orn-Pro-cha-Trp-Pmf] 37 Ac-Phe-[Orn-Pro-cha-Trp-2Ni] 38 Ac-Phe-[Orn-Pro-cha-Trp-Thi] 41 Ph-CH.sub.2-CH.sub.2-CO-[Orn-Pro-cha-Trp-Nle] 42 H-Phe-[Orn-Pro-cha-Trp-Nle] 43 Ac-Lys-Phe-[Orn-Pro-cha-Trp-Nle] 44 H-Phe-[Orn-Ser-cha-Trp-Nle] 51 Ac-Phe-Orn-Pro-cha-Trp-Phe-NH.sub.2 52 Ac-Phe-Orn-Aze-cha-Bta-Phe-NH.sub.2 53 Ac-Phe-Orn-Pro-cha-Bta-2Ni—NH.sub.2 54 Ac-Phe-Orn-Pro-cha-Bta-Cha-NH.sub.2 55 Ac-Phe-Orn-Pip-cha-Trp-Phe-NH.sub.2 56 Ph-CH.sub.2-[Orn-Pro-cha-Trp-Nle] 57 Ph-CH.sub.2-[Orn-Pro-cha-Trp-Phe] 58 Ac-Phe-[Orn-Pro-cha-Trp-1Ni] 59 Ph-CH(OH)—CH.sub.2-CO-[Orn-Pro-cha-Trp-Nle] 61 Ac-Phe-Orn-Pro-cha-Trp-Phe-NH.sub.2 62 Ac-Phe-Orn-Pro-cha-Bta-Phe-NH.sub.2 64 Ac-Phe-Orn-Pro-cha-Trp-2Ni—NH.sub.2 65 Ac-Phe-Orn-Pro-cha-Trp-Cha-NH.sub.2 66 Ac-Thi-Orn-Aze-cha-Bta-Phe-NH.sub.2 67 Ac-Thi-Orn-Pip-cha-Bta-Phe-NH.sub.2 68 Ac-Phe-Orn-Pro-cha-Trp-Eap-NH.sub.2 69 Me.sub.2-Phe-Orn-Pro-cha-Trp-Phe-NH.sub.2 70 Ph.sub.2-CH—CH.sub.2-CO-Orn-Pro-cha-Trp-Phe-NH.sub.2 71 Ac-Ebw-Orn-Pro-cha-Trp-Phe-NH.sub.2 72 Ac-Phe-Orn-Pro-cha-Trp-NH—CH.sub.2-CH.sub.2-Ph 73 Ac-Phe-Orn-Aze-cha-Bta-NH—CH.sub.2-CH.sub.2-Ph 74 H-Phe-Orn-Pro-cha-Trp-Phe-NH.sub.2 75 H-Me-Phe-Orn-Pro-cha-Trp-Phe-NH.sub.2 76 Bu-NH—CO-Phe-Orn-Pro-cha-Trp-Phe-NH.sub.2 77 Ac-Thi-Orn-Pro-cha-Trp-Phe-NH.sub.2 78 Ac-Ebw-Orn-Pro-cha-Trp-Phe-NH.sub.2 79 Ac-Phe-Orn-Ala-cha-Trp-Phe-NH.sub.2 80 Ac-Phe-Orn-Pro-cha-Trp-Thi-NH.sub.2 81 Ac-Phe-Orn-Aze-cha-Pcf-Phe-NH.sub.2 82 Ac-Phe-Orn(Ac)-Pro-cha-Trp-Phe-NH.sub.2 83 Ac-Phe-Orn-Aze-cha-Trp-Phe-NH.sub.2 84 Ac-Phe-Trp-Pro-cha-Trp-Phe-NH.sub.2 85 Ph-NH—CO-Phe-Orn-Pro-cha-Trp-Phe-NH.sub.2 86 Bu-O—CO-Phe-Orn-Pro-cha-Trp-Phe-NH.sub.2 87 Ac-Phe-Lys-Pro-cha-Trp-Phe-NH.sub.2 88 Ac-Phe-Arg-Pro-cha-Trp-Phe-NH.sub.2 89 Ac-Phe-Gln-Pro-cha-Trp-Phe-NH.sub.2 92 Ac-Phe-Orn-Pip-cha-Trp-Phe-NH.sub.2 93 Ac-Phe-Orn-Hyp-cha-Trp-Phe-NH.sub.2 94 Ac-Phe-Orn-Pro-cha-Trp-1Ni-—NH.sub.2 95 Ac-Phe-Orn-Aze-cha-Bta-Phe-NH-Me 96 CH.sub.3-SO.sub.2-Phe-Orn-Aze-cha-Bta-Phe-NH.sub.2 99 Ac-Phe-Orn-Aze-cha-Pff-Phe-NH.sub.2 100 Ac-Phe-Orn-Aze-cha-Mcf-Phe-NH.sub.2 101 Ac-Phe-Orn(Ac)-Aze-cha-Bta-Phe-NH.sub.2 102 Ac-Ebw-Orn-Pro-cha-Trp-Phe-NH.sub.2 103 Ac-Phe-Trp-Pro-cha-Trp-Phe-NH.sub.2 104 Ac-Phe-Arg-Pro-cha-Trp-Phe-NH.sub.2 105 Ac-Phe-Orn-Pip-cha-Trp-Phe-NH.sub.2 106 3PP-Orn-Aze-cha-Bta-Phe-NH.sub.2 107 Ac-Phe-Orn-Tic-cha-Trp-Phe-NH.sub.2 108 Ac-Phe-Orn-Ser-cha-Trp-Phe-NH.sub.2 109 Ac-Phe-Orn-Pro-chg-Trp-Phe-NH.sub.2 110 Ac-Phe-Orn-Pro-hch-Trp-Phe-NH.sub.2 111 Ac-Phe-Orn-Pro-cha-Trp-Phg-NH.sub.2 112 Ac-Phe-Bta-Aze-cha-Bta-Phe-NH.sub.2 113 Ac-Phe-Trp-Pro-cha-Bta-Phe-NH.sub.2 115 Ac-Phe-Orn-Pip-cha-Trp-Phe-OH 116 Ac-Phe-Orn-Tic-cha-Trp-Phe-OH 117 Ac-Phe-Orn-Ser-cha-Trp-Phe-OH 118 Ac-Phe-Orn-Pro-chg-Trp-Phe-OH 119 Ac-Phe-Eec-Pro-cha-Bta-Phe-NH.sub.2 120 Ac-Phe-Nle-Pro-cha-Bta-Phe-NH.sub.2 121 Ac-Phe-Har-Pro-cha-Bta-Phe-NH.sub.2 122 Ac-Phe-Arg-Pro-cha-Bta-Phe-NH.sub.2 123 Ac-Phe-Cys(Acm)-Pro-cha-Bta-Phe-NH.sub.2 124 Ac-Phe-Mpa-Pro-cha-Bta-Phe-NH.sub.2 125 Ac-Eby-Orn-Pro-cha-Bta-Phe-NH.sub.2 126 Ac-Phg-Orn-Pro-cha-Bta-Phe-NH.sub.2 127 Ac-Phe-Paf-Pro-cha-Bta-Phe-NH.sub.2 128 H.sub.2N—CO-Phe-Orn-Pro-cha-Bta-Phe-NH.sub.2 129 Me-O—CO-Phe-Orn-Pro-cha-Bta-Phe-NH.sub.2 130 (—CO—CH.sub.2-NH—CO—)-Phe-Orn-Pro-cha-Bta-Phe-NH.sub.2 132 Ac-Phe-Orn-Pro-hch-Trp-Phe-OH 133 (—CO—CH.sub.2-CH.sub.2-CO—)-Phe-Orn-Pro-cha-Bta-Phe-NH.sub.2 134.sup.tBu-CO-Phe-Orn-Pro-cha-Bta-Phe-NH.sub.2 135 Ac-Lys-Phe-Orn-Aze-cha-Bta-Phe-NH.sub.2 136 Ac-Gly-Phe-Orn-Aze-cha-Bta-Phe-NH.sub.2 137 Ac-Arg-Phe-Orn-Aze-cha-Bta-Phe-NH.sub.2 138 Ac-His-Phe-Orn-Aze-cha-Bta-Phe-NH.sub.2 139 Ac-Ser-Phe-Orn-Aze-cha-Bta-Phe-NH.sub.2 140 Ac-Guf-Phe-Orn-Aze-cha-Bta-Phe-NH.sub.2 141 Ac-Dab-Phe-Orn-Aze-cha-Bta-Phe-NH.sub.2 142 FH.sub.2C—CO-Phe-Orn-Pro-cha-Bta-Phe-NH.sub.2 143 Ac-Phe-Orn(Et.sub.2)-Pro-cha-Trp-Phe-NH.sub.2 144 Ac-Phe-[Orn-Hyp-cha-Trp-Nle] 145 3PP-[Orn-Hyp-cha-Trp-Nle] 146 Ac-Phe-[Orn-Pro-cha-Trp-Tyr] 147 Ac-Phe-[Orn-Pro-omf-Trp-Nle] 149 Ac-Phe-Orn-Pro-hle-Bta-Phe-NH.sub.2 150 Ac-Phe-Arg(CH.sub.2-CH.sub.2)-Pro-cha-Bta-Phe-NH.sub.2 151 Ac-Ala-Phe-Orn-Aze-cha-Bta-Phe-NH2 152 Ac-Arg-Phe-Orn-Aze-cha-Bta-Phe-NH2 153 Ac-Cit-Phe-Orn-Aze-cha-Bta-Phe-NH2 154 Ac-Gly-Phe-Orn-Aze-cha-Bta-Phe-NH2 155 Ac-Gly-Phe-Orn-Aze-chg-Bta-Phe-NH2 156 Ac-Gly-Phe-Orn-Aze-hch-Bta-Phe-NH2 157 Ac-Gly-Thi-Orn-Aze-cha-Bta-Phe-NH2 158 Ac-His-Phe-Orn-Aze-cha-Bta-Phe-NH2 159 Ac-Hyp-Phe-Orn-Aze-cha-Bta-Phe-NH2 160 Ac-Lys-Phe-Orn-Aze-cha-Bta-Phe-NH2 161 Ac-Mff-Orn-Pro-cha-Bta-Phe-NH2 162 Ac-Mff-Orn-Pro-hle-Bta-Phe-NH2 163 Ac-Mff-Orn-Pro-hle-Mcf-Mff-NH2 164 Ac-Mmy-Orn-Pro-hle-Pff-Phe-NH2 165 Ac-NMF-Orn-Pro-cha-Bta-Phe-NH2 166 Ac-Off-Orn-Pro-cha-Bta-Phe-NH2 167 Ac-Off-Orn-Pro-hle-Bta-Phe-NH2 168 Ac-Orn-Phe-Orn-Aze-cha-Bta-Phe-NH2 169 Ac-Pff-Orn-Pro-cha-Bta-Phe-NH2 170 Ac-Pff-Orn-Pro-hle-Bta-Phe-NH2 171 Ac-Pff-Orn-Pro-hle-Mcf-Pff-NH2 172 Ac-Phe-[Cys-Pro-cha-Bta-Phe-Cys]-NH2 173 Ac-Phe-[Orn-Asn-cha-Trp-Nle] 174 Ac-Phe-[Orn-Aze-cha-Trp-Nle] 175 Ac-Phe-[Orn-Chy-cha-Trp-Nle] 176 Ac-Phe-[Orn-HyA-cha-Trp-Phe] 177 Ac-Phe-[Orn-Hyp-hle-Bta-Phe] 178 Ac-Phe-[Orn-Hyp-hle-Mcf-Phe] 179 Ac-Phe-[Orn-Hyp-hle-Pff-Nle] 180 Ac-Phe-[Orn-Hyp-hle-Pff-Phe] 181 Ac-Phe-[Orn-Hyp-hle-Trp-Phe] 182 Ac-Phe-[Orn-Hyp-Mmf-Trp-Nle] 183 Ac-Phe-[Orn-Hyp-Mmf-Trp-Phe] 184 Ac-Phe-[Orn-NMD-cha-Trp-Nle] 185 Ac-Phe-[Orn-Pip-hle-Bta-Phe] 186 Ac-Phe-[Orn-Pro-cha-Pff-Nle] 187 Ac-Phe-[Orn-Pro-cha-Pff-Phe] 188 Ac-Phe-[Orn-Pro-cha-Trp-1Ni] 189 Ac-Phe-[Orn-Pro-cha-Trp-Cha] 190 Ac-Phe-[Orn-Pro-cha-Trp-Chg] 192 Ac-Phe-[Orn-Pro-cha-Trp-Ecr] 193 Ac-Phe-[Orn-Pro-cha-Trp-Leu] 194 Ac-Phe-[Orn-Pro-cha-Trp-nle] 195 Ac-Phe-[Orn-Pro-cha-Trp-Phe] 196 Ac-Phe-[Orn-Pro-hle-Bta-Nle] 197 Ac-Phe-[Orn-Pro-hle-Bta-Phe] 198 Ac-Phe-[Orn-Pro-hle-Pff-Phe] 199 Ac-Phe-[Orn-Pro-hle-Trp-Nle] 200 Ac-Phe-[Orn-Ser-cha-Trp-Nle] 201 Ac-Phe-[Orn-Ser-cha-Trp-Nle] 202 Ac-Phe-[Orn-Ser-hle-Trp-Nle] 203 Ac-Phe-[Orn-Thr-cha-Trp-Nle] 204 Ac-Phe-[Orn-Tic-cha-Trp-Nle] 205 Ac-Phe-[Orn-Tic-cha-Trp-Nle] 206 Ac-Phe-Ala-Pro-cha-Bta-Phe-NH2 207 Ac-Phe-Arg-Pro-hle-Bta-Phe-NH2 208 Ac-Phe-Arg-Pro-hle-Mcf-Phe-NH2 209 Ac-Phe-Cit-Hyp-hle-Bta-Phe-NH2 210 Ac-Phe-Cit-Pro-cha-Bta-Phe-NH2 211 Ac-Phe-Cit-Pro-hle-Bta-Phe-NH2 212 Ac-Phe-Cit-Ser-hle-Bta-Phe-NH2 213 Ac-Phe-Dab-Aze-cha-Bta-Phe-NH2 214 Ac-Phe-Dab-Aze-hle-Bta-Phe-NH2 215 Ac-Phe-Dab-Pro-cha-Bta-Phe-NH2 216 Ac-Phe-Dap-Pro-cha-Bta-Phe-NH2 217 Ac-Phe-Ech-Pro-cha-Bta-Phe-NH2 218 Ac-Phe-Eep-Pro-cha-Bta-Phe-NH2 219 Ac-Phe-Fcn-Aze-cha-Bta-Phe-NH2 220 Ac-Phe-Fcn-Pro-cha-Bta-Phe-NH2 221 Ac-Phe-Fco-Pro-cha-Bta-Phe-NH2 222 Ac-Phe-Fco-Pro-cha-Bta-Phe-NH2 223 Ac-Phe-Fcp-Aze-cha-Bta-Phe-NH2 224 Ac-Phe-Ffa-Aze-cha-Bta-Phe-NH2 225 Ac-Phe-Ffa-Pro-cha-Bta-Phe-NH2 226 Ac-Phe-Ffa-Pro-hle-Bta-Phe-NH2 227 Ac-Phe-G23-Pro-cha-Bta-Phe-NH2 228 Ac-Phe-Guf-Pro-cha-Bta-Phe-NH2 229 Ac-Phe-Har-Aze-cha-Bta-Phe-NH2 230 Ac-Phe-His-Pro-cha-Bta-Phe-NH2 231 Ac-Phe-L22-Pro-cha-Bta-Phe-NH2 232 Ac-Phe-OrA-Pro-cha-Bta-Phe-NH2 233 Ac-Phe-OrE-Pro-cha-Bta-Phe-NH2 234 Ac-Phe-Orn-Aze-hle-Bta-Phe-NH2 235 Ac-Phe-Orn-Chy-cha-Bta-Phe-NH2 236 Ac-Phe-Orn-Chy-hle-Pff-Phe-NH2 237 Ac-Phe-Orn-G24-cha-Bta-Phe-NH2 238 Ac-Phe-Orn-G25-cha-Bta-Phe-NH2 239 Ac-Phe-Orn-G26-cha-Bta-Phe-NH2 240 Ac-Phe-Orn-G27-cha-Bta-Phe-NH2 241 Ac-Phe-Orn-G30-cha-Bta-Phe-NH2 242 Ac-Phe-Orn-G31-cha-Bta-Phe-NH2 243 Ac-Phe-Orn-Hse-cha-Bta-Phe-NH2 244 Ac-Phe-Orn-Hyp-hle-Bta-Phe-NH2 245 Ac-Phe-Orn-Hyp-hle-Pff-Phe-NH2 246 Ac-Phe-Orn-NMA-cha-Bta-Phe-NH2 247 Ac-Phe-Orn-NMS-cha-Bta-Phe-NH2 248 Ac-Phe-Orn-Pro-cha-1Ni-Phe-NH2 249 Ac-Phe-Orn-Pro-cha-Bta-1Ni—NH2 250 Ac-Phe-Orn-Pro-cha-Bta-Bhf-NH2 251 Ac-Phe-Orn-Pro-cha-Bta-Dff-NH2 252 Ac-Phe-Orn-Pro-cha-Bta-Eaa-NH2 253 Ac-Phe-Orn-Pro-cha-Bta-L19 254 Ac-Phe-Orn-Pro-cha-Bta-Mcf-NH2 255 Ac-Phe-Orn-Pro-cha-Bta-Mff-NH2 256 Ac-Phe-Orn-Pro-cha-Bta-NH—CH(CH20H)—CH2-Ph 257 Ac-Phe-Orn-Pro-Cha-Bta-NH—NBn-CO—NH2 258 Ac-Phe-Orn-Pro-cha-Bta-Opa-NH2 259 Ac-Phe-Orn-Pro-cha-Bta-Pcf-NH2 260 Ac-Phe-Orn-Pro-cha-Bta-Pmf-NH2 261 Ac-Phe-Orn-Pro-cha-Bta-Thi-NH2 262 Ac-Phe-Orn-Pro-cha-Otf-Phe-NH2 263 Ac-Phe-Orn-Pro-ctb-Bta-Phe-NH2 264 Ac-Phe-Orn-Pro-ctb-Eaa-Phe-NH2 265 Ac-Phe-Orn-Pro-ctb-Mcf-Phe-NH2 266 Ac-Phe-Orn-Pro-ctb-Pff-Phe-NH2 267 Ac-Phe-Orn-Pro-hch-Trp-Phe-OH 268 Ac-Phe-Orn-Pro-hle-1Ni-Phe-NH2 269 Ac-Phe-Orn-Pro-hle-6FW-Phe-NH2 270 Ac-Phe-Orn-Pro-hle-Bta-1Ni—NH2 271 Ac-Phe-Orn-Pro-hle-Bta-2Ni—NH2 272 Ac-Phe-Orn-Pro-hle-Bta-5Ff-NH2 273 Ac-Phe-Orn-Pro-hle-Bta-Aic-NH2 274 Ac-Phe-Orn-Pro-hle-Bta-Cha-NH2 275 Ac-Phe-Orn-Pro-hle-Bta-Chg-NH2 276 Ac-Phe-Orn-Pro-hle-Bta-Eaa-NH2 277 Ac-Phe-Orn-Pro-hle-Bta-Egy-NH2 278 Ac-Phe-Orn-Pro-hle-Bta-Pcf-NH2 279 Ac-Phe-Orn-Pro-hle-Bta-Pff-NH2 280 Ac-Phe-Orn-Pro-hle-Bta-Phe-NH2 281 Ac-Phe-Orn-Pro-hle-Bta-phe-OH 282 Ac-Phe-Orn-Pro-hle-Bta-Tyr-NH2 283 Ac-Phe-Orn-Pro-hle-Dff-Phe-NH2 284 Ac-Phe-Orn-Pro-hle-Eaa-Phe-NH2 285 Ac-Phe-Orn-Pro-hle-Egc-Phe-NH2 286 Ac-Phe-Orn-Pro-hle-Egy-Phe-NH2 287 Ac-Phe-Orn-Pro-hle-Egz-Phe-NH2 288 Ac-Phe-Orn-Pro-hle-Mcf-2Ni—NH2 289 Ac-Phe-Orn-Pro-hle-Mcf-Cha-NH2 290 Ac-Phe-Orn-Pro-hle-Mcf-Pff-NH2 291 Ac-Phe-Orn-Pro-hle-Mcf-Phe-NH2 292 Ac-Phe-Orn-Pro-hle-Mff-Phe-NH2 293 Ac-Phe-Orn-Pro-hle-Mmy-Phe-NH2 294 Ac-Phe-Orn-Pro-hle-Ocf-Phe-NH2 295 Ac-Phe-Orn-Pro-hle-Off-Phe-NH2 296 Ac-Phe-Orn-Pro-hle-Otf-Phe-NH2 297 Ac-Phe-Orn-Pro-hle-Pff-2Ni—NH2 298 Ac-Phe-Orn-Pro-hle-Pff-Cha-NH2 299 Ac-Phe-Orn-Pro-hle-Pff-Eaa-NH2 300 Ac-Phe-Orn-Pro-hle-Pff-Mmy-NH2 301 Ac-Phe-Orn-Pro-hle-Pff-Pff-NH2 302 Ac-Phe-Orn-Pro-hle-Pff-Phe-NH2 304 Ac-Phe-Orn-Pro-hle-Phe-Phe-NH2 305 Ac-Phe-Orn-Pro-hle-Tff-Phe-NH2 306 Ac-Phe-Orn-Pro-hle-Trp-Phe-NH2 307 Ac-Phe-Orn-Pro-ile-Trp-Phe-NH2 308 Ac-Phe-Orn-Pro-omf-Bta-Phe-NH2 309 Ac-Phe-Orn-Ser-cha-Bta-Phe-NH2 310 Ac-Ser-Phe-Orn-Aze-cha-Bta-Phe-NH2 311 Ac-Thi-[Orn-Pro-hle-Bta-Phe] 312 Ac-Thi-Orn-Pro-cha-Bta-Phe-NH2 313 Ac-Thi-Orn-Pro-cha-Bta-Thi-NH2 314 Ac-Thr-Phe-Orn-Aze-cha-Bta-Phe-NH2 315 Bzl-[Orn-Pro-cha-Bta-Nle] 316 CH3CH2CO-Phe-Orn-Pro-cha-Bta-Phe-NH2 317 Def-[Orn-Ser-hle-Trp-Nle] 318 Eby-Phe-[Orn-Hyp-cha-Trp-Phe] 319 Eth-Phe-[Orn-Pro-hle-Pff-Nle] 320 FAc-Phe-Fib-Aze-cha-Bta-Phe-NH2 321 FAc-Phe-Orn-Aze-cha-Bta-Phe-NH2 322 FAc-Phe-Orn-Pro-cha-Bta-Phe-NH2 323 Fai-Phe-[Orn-Hyp-cha-Trp-Phe] 324 Faz-Orn-Pro-cha-Bta-Phe-NH2 325 Fbi-Phe-[Orn-Pro-cha-Trp-Nle] 326 Fbn-Phe-[Orn-Hyp-cha-Trp-Phe] 327 Fbn-Phe-[Orn-Pro-cha-Trp-Nle] 328 Fbn-Phe-[Orn-Pro-cha-Trp-Nle] 329 Fbn-Phe-Cit-Pro-hle-Bta-Phe-NH2 330 Fbo-Phe-[Orn-Pro-cha-Trp-Nle] 331 Fbp-[Orn-Pro-cha-Trp-Nle] 332 Fci-[Phe-Orn-Hyp-cha-Trp-Phe] 333 Fck-[Phe-Orn-Pro-cha-Trp-Nle] 334 Fck-Phe-[Orn-Pro-cha-Trp-Nle] 335 Fha-Phe-[Orn-Hyp-cha-Trp-Phe] 336 Fhb-[Phe-Orn-Hyp-cha-Trp-Phe] 337 Fhi-Phe-[Orn-Hyp-cha-Trp-Phe] 338 Fhu-Phe-[Orn-Pro-hle-Pff-Nle] 339 Fhu-Phe-Orn-Pro-cha-Bta-Phe-NH2 340 Fid-Phe-Orn-Pro-cha-Bta-Phe-NH2 341 H-Amf-[Orn-Aze-hle-Pff-Nle] 342 H-Bal-Phe-[Orn-Hyp-hle-Trp-Nle] 343 H-Bal-Phe-[Orn-Pro-hle-Pff-Nle] 344 H-Eby-[Orn-Hyp-hle-Trp-Nle] 345 H-Gly-Phe-Orn-Pro-cha-Bta-Phe-NH2 346 H-Nip-Phe-Cit-Pro-hle-Bta-Phe-NH2 347 Hoo-Phe-[Orn-Hyp-hle-Pff-Nle] 348 Hoo-Phe-Cit-Pro-hle-Pff-Phe-NH2 349 Hoo-Phe-Orn-Hyp-hle-Pff-Phe-NH2 350 Hoo-Phe-Orn-Pro-hle-Bta-Phe-NH2 351 Hoo-Phe-Orn-Pro-hle-Mcf-Phe-NH2 352 Hoo-Phe-Orn-Pro-hle-Pff-Phe-NH2 353 H-Phe-[Lys-Hyp-hle-Pff-Nle] 354 H-Phe-[Orn-Hym-hle-Mcf-Nle] 355 H-Phe-[Orn-Hym-hle-Pff-Phe] 356 H-Phe-[Orn-Hyp-cha-Trp-Nle] 357 H-Phe-[Orn-Hyp-cha-Trp-Phe] 358 H-Phe-[Orn-Hyp-ctb-Pff-Nle] 359 H-Phe-[Orn-Hyp-ctb-Trp-Nle] 360 H-Phe-[Orn-Hyp-ctb-Trp-Phe] 361 H-Phe-[Orn-Hyp-hle-Mcf-Leu] 362 H-Phe-[Orn-Hyp-hle-Pff-Chg] 363 H-Phe-[Orn-Hyp-hle-Pff-Hle] 364 H-Phe-[Orn-Hyp-hle-Pff-Leu] 365 H-Phe-[Orn-Hyp-hle-Pff-Nle] 366 H-Phe-[Orn-Hyp-hle-Pff-Phe] 367 H-Phe-[Orn-Hyp-hle-Trp-Hle] 368 H-Phe-[Orn-Hyp-hle-Trp-Leu] 369 H-Phe-[Orn-Hyp-hle-Trp-Nle] 370 H-Phe-[Orn-Hyp-hle-Trp-Nva] 371 H-Phe-[Orn-Hyp-hle-Trp-Phe] 372 H-Phe-[Orn-NMS-cha-Trp-Nle] 373 H-Phe-[Orn-NMS-hle-Pff-Phe] 374 H-Phe-[Orn-Pro-cha-Pff-Nle] 375 H-Phe-[Orn-Pro-cha-Pff-Phe] 376 H-Phe-[Orn-Pro-cha-Trp-Nle] 377 H-Phe-[Orn-Pro-hle-Mcf-Phe] 378 H-Phe-[Orn-Pro-hle-Ocf-Phe] 379 H-Phe-[Orn-Pro-hle-Pff-Nle] 380 H-Phe-[Orn-Pro-hle-Pff-Phe] 381 H-Phe-[Orn-Pro-hle-Trp-Nle] 382 H-Phe-[Orn-Ser-cha-Trp-Nle] 383 H-Phe-[Orn-Ser-cha-Trp-Phe] 384 H-Phe-[Orn-Ser-hle-Eaa-Nle] 385 H-Phe-[Orn-Ser-hle-Mcf-Leu] 386 H-Phe-[Orn-Ser-hle-Ocf-Nle] 387 H-Phe-[Orn-Ser-hle-Pff-Leu] 388 H-Phe-[Orn-Ser-hle-Pff-Nle] 389 H-Phe-[Orn-Ser-hle-Pff-Phe] 390 H-Phe-[Orn-Ser-hle-Trp-Nle] 391 H-Phe-Cit-Pro-hle-Bta-Phe-NH2 392 Ohf-[Orn-Hyp-hle-Trp-Nle] 393 Tmg-Phe-[Orn-Hyp-cha-Trp-Phe].

1. A sustained-release microparticle comprising an antagonist and a biodegradable polymeric matrix, wherein the antagonist is a peptide, peptide-derived, or peptidomimetic complement C5aR antagonist or a pharmaceutically acceptable acid addition salt thereof.

2. The sustained-release microparticle of claim 1, wherein the antagonist is any of the compounds as disclosed in U.S. Ser. No. 10/564,788 or a pharmaceutically acceptable acid addition salt thereof.

3. The sustained-release microparticle of claim 1, wherein the antagonist is JPE1375 or a pharmaceutically acceptable acid addition salt thereof.

4. The sustained-release microparticle of claim 1, wherein the biodegradable polymeric matrix is selected from the group consisting of poly(glycolic acid), poly-D,L-lactic acid, poly-L-lactic acid, copolymers of the foregoing, poly(aliphatic carboxylic acids), copolyoxalates, polycaprolactone, polydioxonone, poly(ortho carbonates), poly(acetals), poly(lactic acid-caprolactone), polyorthoesters, poly(glycolic acid-caprolactone), polyanhydrides and polytyrosine (polypeptide polymers) or “pseudo”-poly(amino acids) polymers.

5. The sustained-release microparticle of claim 1, wherein the antagonist comprises 5 to 10 wt. % of the microparticle.

6. The sustained-release microparticle of claim 1, wherein the antagonist comprises about 2 to 20 wt. % of the microparticle.

7. The sustained-release microparticle of claim 1, wherein the antagonist comprises about 3 to 15 wt. % of the microparticle.

8. The sustained-release microparticle of claim 1, wherein the microparticle ranges in size from 1 to 100 microns.

9. The sustained-release microparticle of claim 1, wherein the microparticle ranges in size from 25 to 65 microns.

10. The sustained-release microparticle of claim 1, wherein the microparticle is formulated in a liquid injection vehicle.

11. The sustained-release microparticle of claim 1, wherein the microparticle is formulated in an aqueous liquid injection vehicle.

12. The sustained-release microparticle of claim 9, wherein the aqueous liquid injection vehicle is selected a physiological solution, an aqueous solution of carboxymethyl cellulose with a surfactant, or a combination thereof.

13. The sustained-release microparticle of, claim 1, wherein the microparticle is comprised in a composition which can be administered to a subject by intramuscular injection, by subcutaneous injection, by injection locally to a disease site, by periocular injection, by intraocular injection, or any combination thereof.

14. A sustained release composition comprising an antagonist which is a peptide or peptidomimetic complement C5aR antagonist, and a biodegradable polymer.

15. The composition of claim 14, wherein the antagonist is any of the compounds as disclosed in U.S. Ser. No. 10/564,788 or a pharmaceutically acceptable acid addition salt thereof.

16. A sustained release composition containing a minimum of 3, 5, 5-10, 6-8, >8 to about 20 weight % of a peptide or peptidomimetic complement C5aR antagonist and a biodegradable polymer.

17. The composition of claim 14, where the biodegradable polymer is PLGA, which has lactide/glycolide ratio of 85:15.

18. The composition of claim 14, wherein the antagonist is JPEI375 or a pharmaceutically acceptable acid addition salt thereof.

19. The sustained release composition of claim 14, wherein the composition aggregates in aqueous media.

20. The sustained release composition of claim 14, wherein the composition has a specific density of less than, greater than, or equal to 1.0.

21. The sustained release composition of claim 14, wherein the composition settles in aqueous media.

22. The sustained release composition of claim 14, wherein the composition is injectable as a suspension through a 27 gauge or smaller syringe needle assembly.

23. The sustained release composition of claim 14, wherein the composition is injectable as a 5%, 10%, 20% 30% 40% weight suspension through a 27 gauge or smaller syringe needle assembly.

24. The sustained release composition of claim 14, wherein the composition provides a minimum of 1 month to 12 months exposure of the therapeutic agent at a therapeutically effective concentration.

25. The sustained release composition of claim 14, wherein the composition provides a 1-2, 2-3, 4-6, 5-6, 6-7, 6-8, 8-10, 10-12 month release of the therapeutic agent.

26. The sustained release composition of claim 14, wherein the composition provides a 1-2, 2-3, 4-6, 5-6, 6-7, 6-8, 8-10, 10-12 month release of the therapeutic agent at a therapeutically effective concentration.

27. A method for treating a disease or condition associated with complement activation, comprising administering to a subject a sustained release composition comprising an antagonist, which is a peptide or peptidomimetic C5aR antagonist to the complement cascade, and a biodegradable polymer.

28. The method of claim 27, wherein the disease or disorder is ophthalmic disease, age related macular degeneration, geographic atropy, diabetic retinopathy, conditions related to ischemic or reperfusion injury, inflammatory disease, systemic inflammatory disease, local inflammatory disease, C5aR mediated inflammatory disease, C5aR mediated ophthalmic inflammatory disease, ophthalmic inflammatory disease or any combination thereof.
